SEQUENCE LISTING (67 sequences)

SEQ ID NO: 1 - protein, 333 aa, synthetic construct
MDKTVLDANLDPLKGKTIGVIGYGNQGRVQATIMRENGLNVIVGNVKDKYYELAKKEGFEVYEIDEAVRRSDVALLLIPDEVMKEVYEKKIAPVLQGKKEFVLDFASGYNVAFGLIRPPKSVDTIMVAPRMVGEGIMDLHKQGKGYPVLLGVKQDASGKAWDYAKAIAKGIGAIPGGIAVISSFEEEALLDLMSEHTWVPILFGAIKACYDIAVKEYGVSPEAALLEFYASGELAEIARLIAEEGIFNQMVHHSTTSQYGTLTRMFKYYDVVRRIVENEAKYIWDGSFAKEWSLEQQAGYPVFYRLWELATQSEMAKAEKELYKLLGRKVKND

SEQ ID NO: 2 - protein, 321 aa, synthetic construct
LKGKTIGVIGYGNQGRVQATIMRENGLNVIVGNVKDKYYELAKKEGFEVYEIDEAVRRSDVALLLIPDEVMKEVYEKKIAPVLQGKKEFVLDFASGYNVAFGLIRPPKSVDTIMVAPRMVGEGIMDLHKQGKGYPVLLGVKQDASGKAWDYAKAIAKGIGAIPGGIAVISSFEEEALLDLMSEHTWVPILFGAIKACYDIAVKEYGVSPEAALLEFYASGELAEIARLIAEEGIFNQMVHHSTTSQYGTLTRMFKYYDVVRRIVENEAKYIWDGSFAKEWSLEQQAGYPVFYRLWELATQSEMAKAEKELYKLLGRKVKND

SEQ ID NO: 3 - protein, 451 aa, synthetic construct
MSATHIKFSKRDEDGKELAGATMELRDSSGKTISTWISDGQVKDFYLYPGKYTFVETAAPDGYEVATAITFTVNEQGQVTVNGKATKGDAHIGSLKGKTIGVIGYGNQGRVQATIMRENGLNVIVGNVKDKYYELAKKEGFEVYEIDEAVRRSDVALLLIPDEVMKEVYEKKIAPVLQGKKEFVLDFASGYNVAFGLIRPPKSVDTIMVAPRMVGEGIMDLHKQGKGYPVLLGVKQDASGKAWDYAKAIAKGIGAIPGGIAVISSFEEEALLDLMSEHTWVPILFGAIKACYDIAVKEYGVSPEAALLEFYASGELAEIARLIAEEGIFNQMVHHSTTSQYGTLTRMFKYYDVVRRIVENEAKYIWDGSFAKEWSLEQQAGYPVFYRLWELATQSEMAKAEKELYKLLGRKVKNDLEVLFQGPWSHPQFEKGGGSGGGSGGGSWSHPQFEK

SEQ ID NO: 4 - protein, 92 aa, synthetic construct
MSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 5 - skipped

SEQ ID NO: 6 - protein, 36 aa, synthetic construct
LEVLFQGPWSHPQFEKGGGSGGGSGGGSWSHPQFEK

SEQ ID NO: 7 - protein, 368 aa, synthetic construct
GGGGGSGGSGSLKGKTIGVIGYGNQGRVQATIMRENGLNVIVGNVKDKYYELAKKEGFEVYEIDEAVRRSDVALLLIPDEVMKEVYEKKIAPVLQGKKEFVLDFASGYNVAFGLIRPPKSVDTIMVAPRMVGEGIMDLHKQGKGYPVLLGVKQDASGKAWDYAKAIAKGIGAIPGGIAVISSFEEEALLDLMSEHTWVPILFGAIKACYDIAVKEYGVSPEAALLEFYASGELAEIARLIAEEGIFNQMVHHSTTSQYGTLTRMFKYYDVVRRIVENEAKYIWDGSFAKEWSLEQQAGYPVFYRLWELATQSEMAKAEKELYKLLGRKVKNDLEVLFQGPWSHPQFEKGGGSGGGSGGGSWSHPQFEK

SEQ ID NO: 8 - protein, 5 aa, synthetic construct
GGGGG

SEQ ID NO: 9 - protein, 6 aa, synthetic construct
SGGSGS

SEQ ID NO: 10 - protein, 1269 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQGGSGGSSIIAYTMSLGAENSVACSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTICSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGYIPEAPRDGQAYVRKDGEWVLLSTFLAHIVMVDAYKPTKLEVLFQGPAGWSHPQFEKGGGSGGGSGGGSWSHPQFEK

SEQ ID NO: 11 - protein, 1192 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQGGSGGSSIIAYTMSLGAENSVACSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTICSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSG

SEQ ID NO: 12 - protein, 26 aa, synthetic construct
YIPEAPRDGQAYVRKDGEWVLLSTFL

SEQ ID NO: 13 - protein, 13 aa, synthetic construct
AHIVMVDAYKPTK

SEQ ID NO: 14 - protein, 1273 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQGGSGGSSIIAYTMSLGAENSVACSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTICSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGYIPEAPRDGQAYVRKDGEWVLLSTFLGGSGGSLPETGGGSGGSLEVLFQGPAGWSHPQFEKGGGSGGGSGGGSWSHPQFEK

SEQ ID NO: 15 - protein, 17 aa, synthetic construct
GGSGGSLPETGGGSGGS

SEQ ID NO: 16 - protein, 6 aa, synthetic construct
GGSGGS

SEQ ID NO: 17-18 - skipped (2 entries)

SEQ ID NO: 19 - protein, 4 aa, synthetic construct
GPGP

SEQ ID NO: 20 - protein, 8 aa, synthetic construct
GGGSGGGS

SEQ ID NO: 21 - skipped

SEQ ID NO: 22 - protein, 102 aa, synthetic construct
EVRIFAGNDPAHTATGSSGISSPTPALTPLMLDEATGKLVVWDGQKAGSAVGILVLPLEGTETALTYYKSGTFATEAIHWPESVDEHKKANAFAGSALSHAA

SEQ ID NO: 23 - protein, 13 aa, synthetic construct
AKFVAAWTLKAAA

SEQ ID NO: 24 - protein, 20 aa, synthetic construct
TCCATGACGTTCCTGACGTT

SEQ ID NO: 25 - protein, 44 aa, synthetic construct
ELAAWCRWGFLLALLPPGIAGRRIRGRILHDGAYSLTLQGLGIH

SEQ ID NO: 26 - protein, 9 aa, synthetic construct
LALLLLDRL

SEQ ID NO: 27 - protein, 9 aa, synthetic construct
LLLDRLNQL

SEQ ID NO: 28 - protein, 5 aa, synthetic construct
  VARIANT 3: note = Any amino acid
LPXTG

SEQ ID NO: 29 - protein, 6 aa, synthetic construct
LPTGAA

SEQ ID NO: 30 - protein, 6 aa, synthetic construct
LPTGGG

SEQ ID NO: 31 - protein, 6 aa, synthetic construct
LPKTGG

SEQ ID NO: 32 - protein, 5 aa, synthetic construct
LPETG

SEQ ID NO: 33 - protein, 5 aa, synthetic construct
  VARIANT 3: note = Any amino acid
LPXTG

SEQ ID NO: 34 - protein, 105 aa, synthetic construct
  VARIANT 3: note = Any amino acid
  VARIANT 6: note = Any amino acid
  VARIANT 7: note = Any amino acid
  VARIANT 8: note = Any amino acid
  VARIANT 9: note = Any amino acid
  VARIANT 10: note = Any amino acid
  VARIANT 11: note = Any amino acid
  VARIANT 12: note = Any amino acid
  VARIANT 13: note = Any amino acid
  VARIANT 14: note = Any amino acid
  VARIANT 15: note = Any amino acid
  VARIANT 16: note = Any amino acid
  VARIANT 17: note = Any amino acid
  VARIANT 18: note = Any amino acid
  VARIANT 19: note = Any amino acid
  VARIANT 20: note = Any amino acid
  VARIANT 21: note = Any amino acid
  VARIANT 22: note = Any amino acid
  VARIANT 23: note = Any amino acid
  VARIANT 24: note = Any amino acid
  VARIANT 25: note = Any amino acid
  VARIANT 26: note = Any amino acid
  VARIANT 27: note = Any amino acid
  VARIANT 28: note = Any amino acid
  VARIANT 29: note = Any amino acid
  VARIANT 30: note = Any amino acid
  VARIANT 31: note = Any amino acid
  VARIANT 32: note = Any amino acid
  VARIANT 33: note = Any amino acid
  VARIANT 34: note = Any amino acid
  VARIANT 35: note = Any amino acid
  VARIANT 36: note = Any amino acid
  VARIANT 37: note = Any amino acid
  VARIANT 38: note = Any amino acid
  VARIANT 39: note = Any amino acid
  VARIANT 40: note = Any amino acid
  VARIANT 41: note = Any amino acid
  VARIANT 42: note = Any amino acid
  VARIANT 43: note = Any amino acid
  VARIANT 44: note = Any amino acid
  VARIANT 45: note = Any amino acid
  VARIANT 46: note = Any amino acid
  VARIANT 47: note = Any amino acid
  VARIANT 48: note = Any amino acid
  VARIANT 49: note = Any amino acid
  VARIANT 50: note = Any amino acid
  VARIANT 51: note = Any amino acid
  VARIANT 52: note = Any amino acid
  VARIANT 53: note = Any amino acid
  VARIANT 54: note = Any amino acid
  VARIANT 55: note = Any amino acid
  VARIANT 56: note = Any amino acid
  VARIANT 57: note = Any amino acid
  VARIANT 58: note = Any amino acid
  VARIANT 59: note = Any amino acid
  VARIANT 60: note = Any amino acid
  VARIANT 61: note = Any amino acid
  VARIANT 62: note = Any amino acid
  VARIANT 63: note = Any amino acid
  VARIANT 64: note = Any amino acid
  VARIANT 65: note = Any amino acid
  VARIANT 66: note = Any amino acid
  VARIANT 67: note = Any amino acid
  VARIANT 68: note = Any amino acid
  VARIANT 69: note = Any amino acid
  VARIANT 70: note = Any amino acid
  VARIANT 71: note = Any amino acid
  VARIANT 72: note = Any amino acid
  VARIANT 73: note = Any amino acid
  VARIANT 74: note = Any amino acid
  VARIANT 75: note = Any amino acid
  VARIANT 76: note = Any amino acid
  VARIANT 77: note = Any amino acid
  VARIANT 78: note = Any amino acid
  VARIANT 79: note = Any amino acid
  VARIANT 80: note = Any amino acid
  VARIANT 81: note = Any amino acid
  VARIANT 82: note = Any amino acid
  VARIANT 83: note = Any amino acid
  VARIANT 84: note = Any amino acid
  VARIANT 85: note = Any amino acid
  VARIANT 86: note = Any amino acid
  VARIANT 87: note = Any amino acid
  VARIANT 88: note = Any amino acid
  VARIANT 89: note = Any amino acid
  VARIANT 90: note = Any amino acid
  VARIANT 91: note = Any amino acid
  VARIANT 92: note = Any amino acid
  VARIANT 93: note = Any amino acid
  VARIANT 94: note = Any amino acid
  VARIANT 95: note = Any amino acid
  VARIANT 96: note = Any amino acid
  VARIANT 97: note = Any amino acid
  VARIANT 98: note = Any amino acid
  VARIANT 99: note = Any amino acid
  VARIANT 100: note = Any amino acid
  VARIANT 101: note = Any amino acid
  VARIANT 102: note = Any amino acid
  VARIANT 103: note = Any amino acid
  VARIANT 104: note = Any amino acid
  VARIANT 105: note = Any amino acid
  VARIANT 6..105: note = This region may encompass 0-100 residues
LPXTGXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 35 - skipped

SEQ ID NO: 36 - protein, 139 aa, synthetic construct
MSYYHHHHHHDYDIPTTENLYFQGAMVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHT

SEQ ID NO: 37 - protein, 119 aa, synthetic construct
YFQGAMVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHT

SEQ ID NO: 38 - protein, 40 aa, synthetic construct
MGSSHHHHHHSSGLVPRGSVPTIVMVDAYKRYKGSGESGK

SEQ ID NO: 39 - protein, 15 aa, synthetic construct
VPTIVMVDAYKRYKS

SEQ ID NO: 40 - protein, 340 aa, synthetic construct
MKLRHLLLTGAALTSFAATTVHGETVVNGAKLTVTKNLDLVNSNALIPNTDFTFKIEPDTTVNEDGNKFKGVALNTPMTKVTYTNSDKGGSNTKTAEFDFSEVTFEKPGVYYYKVTEEKIDKVPGVSYDTTSYTVQVHVLWNEEQQKPVATYIVGYKEGSKVPIQFKNSLDSTTLTVKKKVSGTGGDRSKDFNFGLTLKANQYYKASEKVMIEKTTKGGQAPVQTEASIDQLYHFTLKDGESIKVTNLPVGVDYVVTEDDYKSEKYTTNVEVSPQDGAVKNIAGNSTEQETSTDKDMTITFTNKKDFEVPTGVAMTVAPYIALGIVAVGGALYFVKKKNA

SEQ ID NO: 41 - protein, 674 aa, synthetic construct
MTKSVKFLVLLLVMILPIAGALLIGPISFGAELSKSSIVDKVELDHTTLYQGEMTSIKVSFSDKENQKIKPGDTITLTLPDALVGMTENDSSPRKINLNGLGEVFIYKDHVVATFNEKVESLHNVNGHFSFGIKTLITNSSQPNVIETDFGTATATQRLTIEGVTNTETGQIERDYPFFYKVGDLAGESNQVRWFLNVNLNKSDVTEDISIADRQGSGQQLNKESFTFDIVNDKETKYISLAEFEQQGYGKIDFVTDNDFNLRFYRDKARFTSFIVRYTSTITEAGQHQATFENSYDINYQLNNQDATNEKNTSQVKNVFVEGEASGNQNVEMPTEESLDIPLETIDEWEPKTPTSEQATETSEKTDTTETAESSQPEVHVSPTEEENPDEGETLGTIEPIIPEKPSVTTEENGTTETAESSQPEVHVSPTEEENPDESETLGTIEPIIPEKPSVTTEENGTTETAESSQPEVHVSPAEEENPDESETLGTILPILPEKPSVTTEENGTTETAESSQPEVHVSPTEEENPDESETLGTIAPIIPEKPSVTTEENGITETAESSQPEVHVSPTKEITTTEKKQPSTETTVEKNKNVTSKNQPQILNAPLNTLKNEGSPQLAPQLLSEPIQKLNEANGQRELPKTGTTKTPFMLIAGILASTFAVLGVSYLQIRKN

SEQ ID NO: 42 - protein, 303 aa, synthetic construct
GSARDISSTNVTDLTVSPSKIEDGGKTTVKMTFDDKNGKIQNGDMIKVAWPTSGTVKIEGYSKTVPLTVKGEQVGQAVITPDGATITFNDKVEKLSDVSGFAEFEVQGRNLTQTNTSDDKVATITSGNKSTNVTVHKSEAGTSSVFYYKTGDMLPEDTTHVRWFLNINNEKSYVSKDITIKDQIQGGQQLDLSTLNINVTGTHSNYYSGQSAITDFEKAFPGSKITVDNTKNTIDVTIPQGYGSYNSFSINYKTKITNEQQKEFVNNSQAWYQEHGKEEVNGKSFNHTVHNINANAGIEGTVK

SEQ ID NO: 43 - protein, 102 aa, synthetic construct
MTIEEDSATHIKFSKRDIDGKELAGATMELRDSSGKTISTWISDGQVKDFYLMPGKYTFVETAAPDGYEVATAITFTVNEQGQVTVNGKATKGDAHIVMVDA

SEQ ID NO: 44 - protein, 10 aa, synthetic construct
GGGGSGGGGS

SEQ ID NO: 45 - protein, 15 aa, synthetic construct
QSIALSSLMVAQAIP

SEQ ID NO: 46 - protein, 32 aa, synthetic construct
ILMQYIKANSKFIGIPMGLPQSIALSSLMVAQ

SEQ ID NO: 47 - protein, 7 aa, synthetic construct
ENLYFQG

SEQ ID NO: 48 - protein, 5 aa, synthetic construct
DDDDK

SEQ ID NO: 49 - protein, 4 aa, synthetic construct
IEGR

SEQ ID NO: 50 - protein, 6 aa, synthetic construct
LVPRGS

SEQ ID NO: 51 - protein, 8 aa, synthetic construct
LEVLFQGP

SEQ ID NO: 52-53 - skipped (2 entries)

SEQ ID NO: 54 - protein, 4 aa, synthetic construct
GPGP

SEQ ID NO: 55 - protein, 6 aa, synthetic construct
GGSGGS

SEQ ID NO: 56 - protein, 4 aa, synthetic construct
GGGS

SEQ ID NO: 57 - protein, 8 aa, synthetic construct
GGGSGGGS

SEQ ID NO: 58 - protein, 16 aa, synthetic construct
GGGSGGGSGGGSGGGS

SEQ ID NO: 59 - protein, 1180 aa, synthetic construct
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPILVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQGGSGGSSIIAYTMSLGAENSVACSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTICSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 60 - protein, 1183 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTNGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQGGSGGSSIIAYTMSLGAENSVACSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTICSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 61 - protein, 1184 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQGGSGGSSIIAYTMSLGAENSVACSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQNFKGLTVLPPLLTDEMIAQYTSALLAGTICSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 62 - protein, 1184 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQGGSGGSSIIAYTMSLGAENSVACSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQNFKGLTVLPPLLTDEMIAQYTSALLAGTICSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 63 - protein, 4 aa, synthetic construct
GGGG

SEQ ID NO: 64 - protein, 20 aa, synthetic construct
  VARIANT 1..20: note = This sequence may encompass 1-20 residues
GGGGGGGGGGGGGGGGGGGG

SEQ ID NO: 65 - protein, 6 aa, synthetic construct
HHHHHH

SEQ ID NO: 66 - protein, 20 aa, synthetic construct
GSGSGSGSGSGSGSGSGSGS

SEQ ID NO: 67 - protein, 35 aa, synthetic construct
  VARIANT 1..5: note = This region may encompass 1-5 residues
  VARIANT 6..7: note = This region may encompass 0-2 residues
  VARIANT 8..12: note = This region may encompass 1-5 residues
  VARIANT 13..14: note = This region may encompass 0-2 residues
  VARIANT 15..19: note = This region may encompass 1-5 residues
  VARIANT 20..21: note = This region may encompass 0-2 residues
  VARIANT 22..26: note = This region may encompass 1-5 residues
  VARIANT 27..28: note = This region may encompass 0-2 residues
  VARIANT 29..33: note = This region may encompass 1-5 residues
  VARIANT 34..35: note = This region may encompass 0-2 residues
  VARIANT 1..35: note = This sequence may encompass 1-5 (GaSb) repeating units; wherein a = 1-5 and b = 0-2
GGGGGSSGGGGGSSGGGGGSSGGGGGSSGGGGGSS

(19) United States
(12) Patent Application Publication
(10) Pub. No.: US 2026/0077032 A1
(43) Pub. Date: Mar. 19, 2026

(54) KARI Nanoparticle

(71) Applicant: La Jolla Institute for Immunology, La Jolla, CA (US)

(72) Inventors: Eduardo Olmedillas, La Jolla, CA (US); Erica Ollmann Saphire, La Jolla, CA (US)

(73) Assignee: La Jolla Institute for Immunology, La Jolla, CA (US)

(21) Appl. No.: 18/876,557

(22) PCT Filed: Jun. 30, 2023
(86) PCT No.: PCT/US2023/026748

§ 371 Date: Dec. 18, 2024

Related U.S. Application Data
(60) Provisional application No. 63/357,548, filed on Jun. 30, 2022.
(60) Provisional application No. 63/407,127, filed on Sep. 15, 2022.

(51) Int. Cl. A61K 39/215; A61K 39/00; A61K 39/385; A61K 47/69; C12N 7/00; C12N 9/04; C12N 15/85

(52) U.S. Cl. CPC A61K 39/215; A61K 39/385; A61K 47/6929; C12N 7/00; C12N 9/0006; C12N 15/85; A61K 2039/55555; A61K 2039/6031; C07K 2319/40; C07K 2319/735; C12N 2770/20034; C12N 2800/40; C12Y 101/01086

(57) ABSTRACT

This disclosure provides self-assembling Ketol-acid reductoisomerase (KARI) nanoparticles that are capable of displaying multiple copies of antigens, antibodies and/or proteins or peptides on its surface; as well as nucleic acids encoding recombinant KARI molecules, vectors expressing recombinant KARI molecules, immunogenic polypeptides comprising the self-assembling KARI nanoparticles, methods of producing the self-assembling KART nanoparticles, and methods for eliciting an immune response against an antigen in a subject comprising the self-assembling KARI nanoparticles.

### CROSS-REFERENCE TO RELATED APPLICATIONS

This application is a U.S. National Phase Application under 35 U.S.C. § 371 of International Application No. PCT/US2023/026748, filed Jun. 30, 2023, which claims priority under 35 U.S.C. § 119(e) to U.S. Provisional Patent Application Nos. 63/357,548, filed Jun. 30, 2022, and 63/407,127, filed Sep. 15, 2022, the contents of each of which are hereby incorporated by reference in their entireties.

### STATEMENT OF GOVERNMENT SUPPORT

This invention was made with government support under AI165072 awarded by the National Institutes of Health (NIH). The government has certain rights in the invention.

### INCORPORATION BY REFERENCE OF SEQUENCE LISTING

The instant application contains a Sequence Listing which has been submitted electronically in XML format and is hereby incorporated by reference in its entirety. Said XML copy, created on Oct. 25, 2023, is named 116639-2260.xml and is 109,709 bytes in size.

### BACKGROUND

Substantial progresses have been made in the design of vaccines for countering infections of various pathogens such as viruses. The rational vaccine design strategy consists of the identification of: (1) broadly neutralizing antibodies (bNAbs), (2) the structural analysis of bNAb-antigen complexes, and (3) the structure-based immunogen design and testing.

In the context of immunogen design, significant breakthroughs have been made in the stabilization and redesign of envelope proteins for several viruses and in the multivalent presentation of optimized envelope proteins on virus-like particles (VLPs) or nanoparticles of similar geometry. VLP are ball shaped and about 10-100 nanometers in diameter. This shape and size was determined to correspond to the optimal antigen spacing for B cell activation, which is a minimum of about 20-25 epitopes spaced by 5-10 nm. For this reason, VLPs can elicit strong and long-lasting immune responses due to their large size and dense display of surface antigens.

This rational vaccine design strategy was established in the HIV-1 vaccine field, but it has also provided a potential solution to vaccine development for other viral pathogens and for non-viral disease targets. For example, VLPs vaccines have successfully been developed against cognate viruses (Gardasil® for Human Papillomavirus) or as carriers for foreign antigens. The development of additional effective VLP-type vaccines requires the identification of novel molecules that may self-assemble into nanoparticles.

Despite these substantial progresses in vaccine design, a worldwide spread of SARS-CoV-2 in the human population resulted in the ongoing COVID-19 pandemic that has already caused more than 534 million infections and more than 6.3 million deaths worldwide. Yet, to date, no single universal therapeutic or vaccine has been generated or approved for treating or preventing infection against all known SARS-CoV-2 variants. The ongoing struggles with the SARS-CoV vaccines show that there remain needs in the medical field for more effective and potent vaccine designs for preventing infections of various viral or non-viral pathogens. The present disclosure addresses these unmet needs.

### SUMMARY OF THE DISCLOSURE

One aspect of the present disclosure provides a self-assembling nanoparticle comprising, consisting of, consisting essentially of an amino acid sequence of a recombinant Ketol-acid reductoisomerase (KARI; EC 1.1.1.86) and a tether sequence operably linking the KARI to at least one protein or peptide of interest (e.g., an antigen, antibody, or fragments of each thereof, etc.). In certain embodiments, each protein comprises a capture sequence.

In another aspect, the present disclosure provides an immunogenic conjugate comprising, consisting of, consisting essentially of: a self-assembling nanoparticle comprising the amino acid sequence of a recombinant Ketol-acid reductoisomerase (KARI; EC 1.1.1.86), and a tether sequence; a protein of interest comprising a capture sequence. In some embodiments, the capture sequence and the tether sequence form a covalent bond that operably links the self-assembling nanoparticle to the protein of interest.

In some embodiments, the recombinant KARI comprises, consists of, consists essentially of the amino acid sequence of SEQ ID NO: 1 or 2, or an amino acid sequence having at least about 37%, at least about 40%, at least about 50%, at least about 55%, at least about 65%, at least about 70%, at least about 75%, at least about 80%, at least about 85%, at least about 90%, at least about 95%, or at least about 99% identity to the amino acid sequence of SEQ ID NO: 1 or 2 across the full length of the amino acid sequence, respectively.

In another aspect, the present disclosure provides nucleic acids, expression vectors and compositions, the nucleic acids, expression vectors, or compositions comprising, consisting of, or consisting essentially of the self-assembling nanoparticle or an immunogenic conjugate comprising, consisting of, consisting essentially of the self-assembling KARI nanoparticle. In another aspect, the present disclosure provides methods of using the self-assembling nanoparticles or the immunogenic conjugate.

### BRIEF DESCRIPTION OF THE DRAWINGS

FIGS. 1A-B show the structure and the thermal stability of an archaeal ketol-acid reductoisomerase (KARI). FIG. 1A shows an atomic model of a 2.4A of a KARI Nanoparticle (6KOU) illustrating that in one aspect, the KARI oligomerizes in a 12-mer nanoparticle with a compact core formed by long helix-alpha that provides a strong stability to the nanoparticle; and further illustrates diagram in ribbons where each KARI monomer is shaded differently. FIG. 1B shows the melting temperature (Tm) at which the KARI nanoparticle unfolds (Tm=86° C.), illustrating the high stability of the KARI nanoparticle.

FIGS. 2A-C show the expression and characterization of recombinant KARI fusion protein comprising the wild-type KARI, or KARI operably linked to a SpyCatcher or a SortaseA tag (LPETG (SEQ ID NO: 32)). FIG. 2A shows an SDS-PAGE analysis demonstrating different buffer for the purification of the KARI nanoparticle. Bottom bands show the molecular weight of the monomer to be about 45 KDa, and the top bands show the nanoparticle with a (dodecamer) with a molecular weight of the monomer to be about 540Kda, formed and cross-linked with glutaraldehyde. FIG. 2B shows the relative expression of the KARI Nanoparticle in one liter of bacteria in mg/L. FIG. 2C shows a Cryo_EM volume of KARI nanoparticle linked with SpyTag. Each KARI monomer is shaded differently.

FIGS. 3A-C show schematic representations illustrating the VFLIP-KARI formation and the relative expression of VFLIP tagged. FIG. 3A shows a schematic illustrating the linear sequence of a plasmid encoding a representative nanoparticle (KARI) and the SpyCatcher expressed in bacteria and the linear sequence of a plasmid encoding VFLIP-SARS-CoV-2 spike linked to the SpyTag in a mammalian cell line. FIG. 3B shows an atomic model of the resulting VFLIP-SARS-CoV-2 nanoparticle operably linked (irreversibly conjugated) via SpyTag/SpyCatcher technology. Just one out of 12 spikes is shown. FIG. 3C shows the relative expression of the wild-type and tagged VFLIP expressed in one liter of mammalian cells.

FIGS. 4A-B show a schematic representation illustrating the VFLIP SARS-CoV-2_S2subunit/Nucleoprotein T cell-epitope/KARI formation. FIG. 4A shows a schematic illustrating the linear sequence of a plasmid encoding the nanoparticle (KARI) fused to a Nucleoprotein T cell epitope and the SpyCatcher expressed in bacteria; and the linear sequence of a plasmid encoding VFLIP-SARS-CoV-2 and SpyTag fusion protein expressed in a mammalian cell line (e.g., CHO cell). FIG. 4B shows an atomic model of the resulting VFLIP-SARS-CoV-2 nanoparticle operably linked (irreversibly conjugated) via SpyTag/SpyCatcher. Just one out of 12 spikes is shown.

FIGS. 5A-E show the characterization of a recombinant KARI fused to an entire SARS-CoV-2 Nucleoprotein domains. FIG. 5A shows relative expression of KARI fused to the N-Terminal Domain (NTD, RNA binding domain) and C-Terminal Domain (CTD, dimerization domain) domains of the SARS-CoV-2 Nucleoprotein, illustrating that the NTD domain yields was 5 times higher than the wild-type protein. The NTD mutant R92E is a nucleoprotein mutant that cannot bind RNA. FIG. 5B shows a size exclusion chromatogram of KARI on a Superdex S6i column. The pick of the 15 ml shows the self-assembled nanoparticle. FIG. 5C shows an SDS-PAGE gel of KARI of FIG. 5B with (+) or without (−) glutaraldehyde as cross-linker. The fraction that elutes in 15 ml in FIG. 5B, after cross-linking it has a molecular weight compatible with a self-assembled 12-meric nanoparticle with 12 SpyCatcher tags (770 kDa). FIG. 5D shows an SDS-PAGE gel of KARI with (+) or without (−) glutaraldehyde as cross-linker which contain the NTD (RNA binding domain) and CTD (dimerization domain) domains of the SARS-CoV-2 Nucleoprotein; and illustrates that KARI_NTD and KARI_NTD_R92E were perfect self-assembled nanoparticles while KARI_CTD neither the yield nor the size were compatible with a well-formed nanoparticle. FIG. 5E shows representative 2D classes from negative staining electron microscopy.

FIGS. 6A-B show the binding of two anti-nucleoprotein-N_NTD mAbs. The Binding kinetic measured in Octet shows that two mAbs (Ab6 and Ab7) specific for the NTD domain of the SARS-CoV-w Nucleoprotein, bound the domain in the context of the surface of KARI for both the NTD wild-type (FIG. 6A) and the NTD R92E mutant (FIG. 6B). An anti-CDT_N Ab (Ab14) was used as a negative control.

FIG. 7A-B show the successful binding between KARI and VFLIP. FIG. 7A shows a representative negative staining EM micrograph of KARI surrounded by 12 VLIP forming floret-like structures. In the bottom there are 10 different 2D classes of the resulting florets. FIG. 7B shows a model of the formed florets, with just one VFLIP spike variant (monovalent nanoparticle) or with three different variants of concern (multivalent nanoparticle).

### DETAILED DESCRIPTION

### Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary ofBiochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1′ ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this invention are provided herein.

Throughout and within this disclosure technical and patent publications are referenced to more fully describe the state of the art, some of which are referenced by an Arabic number or first author name and date. The full bibliographic citation for each reference is provided in the “References” section, immediately preceding the claims. All technical and patent publications are incorporated herein by reference to more fully describe the state of the art.

Unless otherwise noted, the expression “a least” or “at least one of” as used herein includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

Unless otherwise noted, the order of steps or order for performing certain actions is immaterial so long as the present invention remain operable. Moreover, two or more steps or actions may be conducted simultaneously.

Unless otherwise noted, the use of any and all examples, or exemplary language herein, for example, “such as” or “including,” is intended merely to illustrate better the present invention and does not pose a limitation on the scope of the invention. No language in the specification should be construed as indicating any non-claimed element as essential to the practice of the present invention.

As used herein, the term “adjuvant” refers to a compound that, when used in combination with a specific immunogen in a formulation, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses.

As used herein, the term “administering” refers to oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

As used herein, the term “ameliorating” refers to any therapeutically beneficial result in the treatment of a disease state, e.g., viral infection, including prophylaxis, lessening in the severity or progression, remission, or cure thereof.

As used herein the term “amino acid” refers to either natural and/or unnatural or synthetic amino acids, including glycine and both the D or L optical isomers, and amino acid analogs and peptidomimetics.

As used herein, the term “antigen” refers to a mutant SARS-CoV-2 spike protein containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides, which include modifications, such as deletions, additions, and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts, which produce the antigens.

As used herein, the term “antigenic formulation” or “antigenic composition” refers to a preparation which, when administered to a vertebrate, e.g., a mammal, will induce an immune response.

As used herein, the term “capture sequence” refers to a polypeptide fused to an KARI or protein of interest (“POI”) with which a tethering sequence forms a covalent peptide linkage, for example SpyCatcher for the SpyTag tethering sequence. The capture sequence may be fused to the recombinant KARI or POI at the N- or C-terminus of such proteins or polypeptides or in an internal loop. Particularly, a spacer sequence (e.g., a glycine/serine rich spacer) may flank the capture sequence to enhance accessibility for reaction. The spacer may further include a site for specific proteolysis (e.g., by Factor X, thrombin, enterokinase, tobacco etch virus Nia protease, rhinovirus 3C protease or trypsin), allowing specific release from a capture sequence.

As used herein, the term “co-administer” refers to a compound or composition described herein that is administered at the same time, just prior to, or just after the administration of one or more additional therapies. The compounds provided herein can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols. The preparations may also be combined with inhaled mucolytics (e.g., rhDNase, as known in the art) or with inhaled bronchodilators (short or long acting beta agonists, short or long acting anticholinergics), inhaled corticosteroids, or inhaled antibiotics to improve the efficacy of these drugs by providing additive or synergistic effects. The compositions of the present invention can be delivered transdermally, by a topical route, formulated as applicator sticks, solutions, suspensions, emulsions, gels, creams, ointments, nanoparticles, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see e.g., Gao Pharm. Res. 12:857-863, 1995); or, as microspheres for oral administration (see e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See e.g., Al-Muhammed, J. Microencapsul. 13:293-306, 1996; Chonn, Curr. Opin. Biotechnol. 6:698-708, 1995; Ostro, Am. J. Hosp. Pharm. 46: 1576-1587, 1989).

As used herein the term “display vector set” refers to particular combinations of expression vectors (which provide GC1 constructs) and helper vectors (which provide GC2 constructs). The co-expression of the GC1 and GC2 result in the display of the protein of interest on the surface of an expression system.

As used herein, the term “epitope” refers to an antigenic determinant. These are particular chemical groups or peptide sequences on a molecule that are antigenic, such that they elicit a specific immune response, for example, an epitope is the region of an antigen to which B and/or T cells respond. Epitopes can be formed both from contiguous amino acids or noncontiguous amino acids juxtaposed by tertiary folding of a protein.

As used herein, the term “effective dose” refers to that amount of one or more compositions and/or nanoparticles of the disclosure sufficient to induce immunity, to prevent and/or ameliorate an infection or to reduce at least one symptom of an infection and/or to enhance the efficacy of another dose of a composition and/or nanoparticle. An effective dose may refer to the amount of a composition and/or nanoparticle sufficient to delay or minimize the onset of an infection. An effective dose may also refer to the amount of a composition and/or nanoparticle that provides a therapeutic benefit in the treatment or management of an infection. Further, an effective dose is the amount with respect to a composition and/or nanoparticle of the disclosure alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and***/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

As used herein, the term “eukaryotic system” refers to eukaryotic cells including cells of animal, plants, fungi and protists, and eukaryotic viruses such as retrovirus, adenovirus, baculovirus. As used herein, the term “expression systems” refers collectively to prokaryotic and eukaryotic systems.

As used herein, the term “expression cassette” refers to a functional unit that is built in a vector for the purpose of expressing recombinant proteins/peptides. An expression cassette includes a promoter or promoters, a transcription terminator sequence, a ribosome binding site or ribosome binding sites, and the cDNA encoding a tether sequence or a capture sequence. Other genetic components can be added to an expression cassette, depending on the expression system (e.g., enhancers and polyadenylation signals for eukaryotic expression systems).

As used herein the term “expression vector” means a polynucleotide which, when introduced into an appropriate host cell, can be transcribed and translated into a polypeptide(s). In some embodiments, the term “expression vector,” refers to vectors that direct the soluble expression of proteins of interest fused in frame with a tether sequence which is characterized by an ability to form a peptide linkage to capture sequence with a capture sequence produced by a helper vector as disclosed herein. The protein of interest (e.g., an antigen) may also be fused in frame with a capture sequence, which is characterized by an ability to form a peptide linkage to tether sequence with a tether sequence produced by a helper vector as disclosed herein.

As used herein, the term “helper vector” refers to a genetic system, or host cell-specific vector designed to produce fusion proteins comprising a recombinant KARI fused in frame with a capture sequence. The recombinant KARI may also be fused in frame with a tether sequence. Helper vectors can be introduced into expression systems, in combination with an expression vector, transiently by co-transformation, permanently by integration into host genome, or by viral or phage infection of the host cells.

As used herein the term “host cell” includes an individual cell or cell culture which can be, or has been, a recipient for the disclosed vectors. Host cells include progeny of a single host cell. The progeny may not necessarily be completely identical to the original parent cell due to natural, accidental, or deliberate mutation.

As used herein, the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interferons, interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as the composition and/or nanoparticle of the disclosure or can be administered separately. Either the protein or an expression vector encoding the protein can be administered to produce an immunostimulatory effect.

As used herein, the term “protective immune response” or “protective response” refers to an immune response mediated by antibodies against an infectious agent, which is exhibited by a vertebrate (e.g., a human), which prevents or ameliorates an infection or reduces at least one symptom thereof. Composition and/or nanoparticle of the disclosure can stimulate the production of antibodies that, for example, neutralize infectious agents, blocks infectious agents from entering cells, blocks replication of said infectious agents, and/or protect host cells from infection and destruction. The term can also refer to an immune response that is mediated by T-lymphocytes and/or other white blood cells against an infectious agent, exhibited by a vertebrate (e.g., a human) or reduces at least one symptom thereof.

As used herein, the term “immune response” refers to an integrated bodily response to an antigen and in the present context preferably refers to an adaptive immune response including a cellular immune response. The immune response may be protective/preventive/prophylactic and/or therapeutic.

As used herein, the term “immunogenic composition” refers to a preparation which, when administered to a vertebrate, especially a bird or a mammal, will induce an immune response.

As used herein, the term “immunogens” or “antigens” refer to substances such as proteins, peptides, peptides, nucleic acids that are capable of eliciting an immune response. Both terms also encompass epitopes and are used interchangeably.

As used herein, the terms “immunization” or “vaccine” are used interchangeably to refer to a formulation which contains one or more of the composition and/or nanoparticle of the present disclosure, which is in a form that is capable of being administered to a vertebrate and which induces a protective immune response sufficient to induce immunity to prevent and/or ameliorate an infection and/or to reduce at least one symptom of an infection and/or to enhance the efficacy of another dose of the composition and/or nanoparticle. Typically, the vaccine comprises a conventional saline or buffered aqueous solution medium in which the composition of the present disclosure is suspended or dissolved. In this form, the composition of the present disclosure can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

As used herein, the term “inducing an immune response” may mean that there was no immune response against a particular antigen before induction, but it may also mean that there was a certain level of immune response against a particular antigen before induction and after induction said immune response is enhanced. Thus, “inducing an immune response” also includes “enhancing an immune response”. In one aspect when the antigen is a cancer antigen or neoantigen, after inducing an immune response in a subject, said subject is protected from developing a disease such as a cancer disease or the disease condition is ameliorated by inducing an immune response. For example, an immune response against a tumor expressed antigen may be induced in a patient having cancer or in a subject being at risk of developing cancer. Inducing an immune response in this case may mean that the disease condition of the subject is ameliorated, that the subject does not develop metastases, or that the subject being at risk of developing a cancer disease does not develop a cancer disease.

As used herein, the term “in vivo” refers to processes that occur in a living organism.

As used herein the term “mammal” or “subject” or “patient” includes both humans and non-humans and include but is not limited to humans, non-human primates (e.g., chimpanzees, monkeys, baboons), canines, felines, mice, rats (e.g., cotton rats), bovines (e.g., calves), equines, porcines, guinea pigs, ferrets and hamsters.

As used herein, the term “nanoparticles” refer to any particles, which are between 1 and 100 nanometers in size. The present disclosure includes formulations comprising the mutant coronavirus spike proteins of the present disclosure formed into nanoparticles or microparticles. In one example, nanoparticles or microparticles are formed with a protein and/or into a polymer matrix. The polymer matrix can be made with, e.g., poly (L-gly colic acid) (PLGA), polyglycolic acid (PGA), polylactic acid (PLA), poly(L-lactic acid) (PLLA), poly(epsilon-Caprolactone) PCL, Poly(methyl vinyl ether-co-maleic anhydride), polyglycolide, poly-L-lactide, poly-D-lactide, poly(amino acids), polyethyleneglycol PEG), polydioxanone, polycaprolactone, polygluconate, polylactic acid-polyethylene oxide copolymers, polyorthoesters, polyhydroxybutyrate, polyanhydride, polyphosphoester, poly(alpha-hydroxy acid), ferritin, chitosan, alginate, collagen, dextran, polyester, cellulose, carboxymethyl cellulose, modified cellulose, collagen, or combinations thereof. In some examples, the nanoparticles are partially or fully biodegradable.

As used herein, the term “operably linked” refers to an arrangement of elements where the components so described are configured so as to perform their usual function. Thus, a given promoter operably linked to a coding sequence is capable of effecting the expression of the coding sequence when active. The promoter need not be contiguous with the coding sequence, so long as it functions to direct the expression thereof. Thus, for example, intervening untranslated yet transcribed sequences can be present between the promoter sequence and the coding sequence, and the promoter sequence can still be considered “operably linked” to the coding sequence.

As used herein, the terms “pharmaceutically acceptable” or “pharmacologically acceptable” refer to a material which is neither biologically nor otherwise undesirable, i.e., the material may be administered to an individual in a formulation or composition without causing any unacceptable biological effects or interacting in a deleterious manner with any of the components of the composition in which it is contained. In some embodiments, a pharmaceutical composition will generally comprise agents for buffering and preservation in storage, and can include buffers and carriers for appropriate delivery, depending on the route of administration.

As used herein, the term “pharmaceutically acceptable excipient” or “pharmaceutically acceptable carrier” refers to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

As used herein, the term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

As used herein, the term “pharmaceutically acceptable vaccine” refers to a formulation which contains a fusion protein, or nanoparticles of the present invention, which is in a form that is capable of being administered to a vertebrate and which induces a protective immune response sufficient to induce immunity to prevent and/or ameliorate an infection or disease, and/or to reduce at least one symptom of an infection or disease, and/or to enhance the efficacy of another dose of a fusion protein or nanoparticle. In one embodiment, to a formulation which contains a fusion protein, or nanoparticle, of the present invention. Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

As used herein, the term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared. In one aspect, the percent identity intends the specified percentage of nucleotides or amino acid residues that are the same when aligned for maximum correspondence across the full length of the nucleotide or amino acid sequence when run under BLAST.

“Percentage of sequence identity” is determined by comparing two optimally aligned sequences over a comparison window, where the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

As used herein, the term a “comparison window” includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'L Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see e.g., Ausubel el al., Current Protocols in Molecular Biology (1995 supplement)).

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Set. USA 89: 10915) alignments (B) of 50, expectation (E) of 10, M=5, N=−4, and a comparison of both strands.

The BLAST algorithm also performs a statistical analysis of the similarity between two sequences (see e.g., Karlin and Altschul (1993) Proc. Natl. Acad. Set. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

As used herein, the term “plurality” refers to two or more of a particular element. For example, two or more polypeptides (e.g., antigens). In certain embodiments, a plurality may refer to 2, 3, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 220, 230, 240, 250, 260, 270, 280, 290, 300 or more elements. In certain embodiments, a plurality may refer to 2 to 180, or 5 to 180, or 10 to 180, or 20 to 180, or 30 to 180, or 40 to 180, or 50 to 180, or 60 to 180, or 70 to 180, or 80 to 180, or 90 to 180, or 100 to 180, or 110 to 180, or 120 to 180, or 130 to 180, or 140 to 180, or 150 to 180, or 160 to 180, or 170 to 180, of a given element (e.g., polypeptide). In certain embodiments, a plurality may refer to 2 to 120, or 5 to 120, or 10 to 120, or 20 to 120, or 30 to 120, or 40 to 120, or 50 to 120, or 60 to 120, or 70 to 120, or 80 to 120, or 90 to 120, or 100 to 120, or 110 to 120, of a given element (e.g., polypeptide). In certain embodiments, a plurality may refer to 2 to 60, or 5 to 60, or 10 to 60, or 20 to 60, or 30 to 60, or 40 to 60 or 50 to 60 of a given element (e.g., polypeptide). In certain embodiments, a plurality refers to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 or more of a given element. Additionally, the elements within a plurality may be each independently selected, and therefore, may be the same or different. For example, a KARI nanoparticle as described herein may comprise a plurality of antigens, which are the same (i.e., the KARI nanoparticle displays a single type of antigen). In other embodiments, the KARI nanoparticle may comprise a plurality of antigens, which are different (e.g., the KARI nanoparticle may display a mixture of antigens). A plurality of antigens may be linked to a single monomer of the KARI nanoparticle.

As used herein, the terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the nucleotide polymer.

As used herein the terms “polypeptide”, “peptide”, and “protein,” are used interchangeably herein to refer to polymers of amino acids of any length. A protein of interest may, thus, be a polypeptide, peptide, or protein.

As used herein, the term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration. The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

As used herein, the term “prokaryotic system” refers to prokaryotic cells such as bacterial cells or prokaryotic viruses, prokaryotic phages or bacterial spores.

As used herein, a “protein of interest” or “POI” is any desired polypeptide, peptide, or protein. Non-limiting examples of protein of interest include antibodies, for example full length antibodies, antibody fragments, single chain antibodies (e.g., scFv, scFab), or single domain antibodies, protein scaffolds (e.g., based on fibronectin III, cystatin, lipocalins, Ankyrin repeat domains, Z domain of protein A and others), hormones, interleukins, antigens for the development of vaccines, enzymes, etc. Other examples include, and are not limited to: human growth hormone (hGH), N-methionyl human growth hormone, bovine growth hormone, parathyroid hormone, thyroxine, insulin A-chain, insulin B-chain, proinsulin, relaxin A-chain, relaxin B-chain, prorelaxin, glycoprotein hormones such as follicle stimulating hormones (FSH), thyroid stimulating hormone (TSH), and leutinizing hormone (LH), glycoprotein hormone receptors, calcitonin, glucagon, factor VIII, an antibody, lung surfactant, urokinase, streptokinase, human tissue-type plasminogen activator (t-PA), bombesin, factor IX, thrombin, hemopoietic growth factor, tumor necrosis factor-alpha and -beta, enkephalinase, human serum albumin, mullerian-inhibiting substance, mouse gonadotropin-associated peptide, a microbial protein, such as betalactamase, tissue factor protein, inhibin, activin, vascular endothelial growth factor, receptors for hormones or growth factors; integrin, thrombopoietin, protein A or D, rheumatoid factors, nerve growth factors such as NGF-b, platelet-growth factor, transforming growth factors (TGF) such as TGF-alpha and TGF-beta, insulin-like growth factor-I and -II, insulin-like growth factor binding proteins, CD-4, DNase, latency associated peptide, erythropoietin, osteoinductive factors, interferons such as interferon-alpha, -beta, and -gamma, colony stimulating factors (CSFs) such as M-CSF, GF-CSF, and G-CSF, interleukins (ILs) such as IL-1, IL-2, IL-3, IL-4, superoxide dismutase; decay accelerating factor, viral antigen, HIV envelope proteins such as GP120, GP140, atrial natriuretic peptides A, B or C, immunoglobulins, and fragments of any of the above-listed proteins.

As used herein, the term “recombinant” refers to a polynucleotide that encodes a mutant or engineered protein (e.g., KARI or SARS-CoV-2 spike protein) whether from a bacterial, viral prokaryotic or eukaryotic viral genome, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

As used herein, the term “self-assembling molecule” refers to a molecule that undergoes spontaneous or induced assembly into defined, stable, noncovalently bonded assemblies that are held together by intermolecular forces. Self-assembling molecules include protein, peptides, nucleic acids, virus-like particles, lipids and carbohydrates. Non-limiting examples of self-assembling molecules include ferritin, heat shock protein, DSP, lumazine synthase, and DNA.

Techniques for determining amino acid sequence “similarity” are well known in the art. In general, “similarity” means the exact amino acid to amino acid comparison of two or more polypeptides at the appropriate place, where amino acids are identical or possess similar chemical and/or physical properties such as charge or hydrophobicity.

As used herein, the term “subject” refers to any member of the subphylum chordata, including, but not limited to, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

As used herein, the term “sufficient amount” or “amount sufficient to” means an amount sufficient to produce a desired effect, e.g., an amount sufficient to inhibit viral fusion to a cell.

As used herein, the term “tether sequence” or “tethering sequence” relate to a sequence that is attached to a protein of interest (POI) or the recombinant KARI and that facilitates the formation of a covalent linkage to a capture moiety expressed either on the surface of a biological entity or fused to the POI. Non-limiting examples of tethering sequences include SpyTag sequences, including SpyTag002, SnoopTag sequences, Sortase motifs, a C-peptide, butelase substrates, and peptiligase substrates. The tether sequence may be fused to POI or anchor protein at the N- or C-terminus of such proteins or polypeptides or in an internal loop. Particularly, a spacer sequence (e.g., a glycine/serine rich spacer) may flank the tether sequence in order to enhance accessibility for reaction. The spacer may further include a site for specific proteolysis (e.g., by Factor X, thrombin, enterokinase, tobacco etch virus (TEV) Ma protease, rhinovirus 3C protease or trypsin), allowing specific release from a tether sequence.

As used herein, the term “therapeutically effective amount” is an amount that is effective to ameliorate a symptom of a disease. A therapeutically effective amount can be a “prophylactically effective amount” as prophylaxis can be considered therapy.

As used herein, the term “treatment” refers to any of (i) the prevention of infection or reinfection with SARS-CoV-2, as in a traditional vaccine, (ii) the reduction or elimination of symptoms, and (iii) the substantial or complete elimination of the pathogen in question. Treatment may be provided prophylactically (prior to infection) or therapeutically (following infection).

As used herein, the term “vaccine” refers to a formulation which contains the fusion proteins or nanoparticles of the present invention, which is in a form that is capable of being administered to a vertebrate and which induces a protective immune response sufficient to induce immunity to prevent and/or ameliorate an infection and/or to reduce at least one symptom of an infection and/or to enhance the efficacy of another dose of the fusion proteins or nanoparticles. Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses. Vaccines can be administered in conjunction with an adjuvant.

In some embodiments, “vaccine” refers to a composition that can provide active acquired immunity to and/or therapeutic effect (e.g., treatment) of a particular disease or a pathogen. A vaccine typically contains one or more agents that can induce an immune response in a subject against a pathogen or disease, i.e. a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g., preventing or ameliorating the effects of a future infection by any natural or pathogen, or of an anticipated occurrence of cancer in a predisposed subject) or therapeutic (e.g., treating cancer or infection in a subject who has been diagnosed with the cancer or infection). The administration of vaccines is referred to vaccination. In embodiments, a vaccine composition can provide nucleic acid, e.g., mRNA that encodes antigenic molecules (e.g., peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g., one or more peptides that are known to be expressed in the pathogen (e.g., pathogenic bacterium or virus). Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

As used herein, the term “vector” refers to a nucleic acid capable of transferring gene sequences to target cells (e.g., bacterial plasmid vectors, viral vectors, non-viral vectors, particulate carriers, and liposomes). Typically, “vector construct,” “expression vector,” and “gene transfer vector,” mean any nucleic acid construct capable of directing the expression of one or more sequences of interest in a host cell. Thus, the term includes cloning and expression vehicles, as well as viral vectors. The term is used interchangeable with the terms “nucleic acid expression vector” and “expression cassette.” Many suitable expression systems are commercially available, including, for example, the following: baculovirus expression (Reilly, P. R., et al., BACULO VIRUS EXPRESSION VECTORS: A LABORATORY MANUAL (1992); Beames, et al., Biotechniques 11:378 (1991); Pharmingen; Clontech, Palo Alto, Calif.)), vaccinia expression systems (Earl, P. L., et al., “Expression of proteins in mammalian cells using vaccinia” In Current Protocols in Molecular Biology (F. M. Ausubel, et al. Eds.), Greene Publishing Associates & Wiley Interscience, New York (1991); Moss, B., et al., U.S. Pat. No. 5,135,855, issued Aug. 4, 1992), expression in bacteria (Ausubel, F. M., et al., CURRENT PROTOCOLS IN MOLECULAR BIOLOGY, John Wiley and Sons, Inc., Media Pa.; Clontech), expression in yeast (Rosenberg, S. and Tekamp-Olson, P., U.S. Pat. No. RE35,749, issued, Mar. 17, 1998, herein incorporated by reference; Shuster, J. R., U.S. Pat. No. 5,629,203, issued May 13, 1997, herein incorporated by reference; Gellissen, G., et al., Antonie Van Leeuwenhoek, 62(1-2):79-93 (1992); Romanos, M. A., et al., Yeast 8(6):423-488 (1992); Goeddel, D. V., Methods in Enzymology 185 (1990); Guthrie, C., and G. R. Fink, Methods in Enzymology 194 (1991)), expression in mammalian cells (Clontech; Gibco-BRL, Ground Island, N.Y.; e.g., Chinese hamster ovary (CHO) cell lines (Haynes, J., et al., Nuc. Acid. Res. 11:687-706 (1983); 1983, Lau, Y. F., et al., Mol. Cell. Biol. 4: 1469-1475 (1984); Kaufman, R. J., “Selection and coamplification of heterologous genes in mammalian cells,” in Methods in Enzymology, vol. 185, pp 537-566. Academic Press, Inc., San Diego Calif. (1991)), and expression in plant cells (plant cloning vectors, Clontech Laboratories, Inc., Palo-Alto, Calif., and Pharmacia LKB Biotechnology, Inc., Pistcataway, N.J.; Hood, E., et al., J. Bacteriol. 168: 1291-1301 (1986); Nagel, R., et al., FEMS Microbiol. Lett. 67:325 (1990); An, et al., “Binary Vectors”, and others in Plant Molecular Biology Manual A3: 1-19 (1988); Miki, B. L. A., et al., pp. 249-265, and others in Plant DNA Infectious Agents (Hohn, T., et al., eds.) Springer-Verlag, Wien, Austria, (1987); Plant Molecular Biology: Essential Techniques, P. G. Jones and J. M. Sutton, New York, J. Wiley, 1997; Miglani, Gurbachan Dictionary of Plant Genetics and Molecular Biology, New York, Food Products Press, 1998; Henry, R. J., Practical Applications of Plant Molecular Biology, New York, Chapman & Hall, 1997), relevant portions of any of the above are incorporated herein by reference.

As used herein, the term “VFLIP” includes any of the recombinant or naturally occurring forms of a coronavirus spike protein, or variants or homologs thereof that maintain coronavirus Spike protein activity (e.g., within at least 50%, 80%, 90%, 95%, 96%, 97%, 98%, 99% or 100% activity compared to coronavirus Spike Protein). In aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring coronavirus Spike protein polypeptide. In embodiments, coronavirus Spike protein is the protein as identified by the UniProt reference number PODTC2, or a variant, homolog, or functional fragment thereof. The mutant coronavirus spike protein includes the amino acid sequence of one of SEQ ID NOs: 1-33 disclosed in WO2022087255A2, or SEQ ID NOs: 39-42 disclosed in pending PCT patent application No. PCT/US2022/053783, both of which are incorporated herein by reference in their entirety. In some embodiments, the mutant coronavirus spike protein comprises the amino acid sequence of one of SEQ ID NO 11 or 14 is a VFLIP.

In some embodiments, the VFLIP comprises one or more mutations in the coronavirus protein. In some embodiments, a VFLIP can comprise the amino acid sequence of SEQ ID NO: 59, SEQ ID NO: 60, SEQ ID NO: 61, SEQ ID NO: 62, or any variant thereof.

In some embodiments, a VFLIP can be a VFLIP_Omicron (Omicron (e.g., B.1.1.529)). Exemplary mutations and an exemplary amino acid sequence of a VFLIP_Omicron (Omicron (B.1.1.529)) is shown in SEQ ID NO: 59 below. Mutations are in Bold, Linker in bold underlined, additional mutations in italics. (SEQ ID NO:59)

QCVNLTTRTQLPPAYTNSFTRGVYYPDKVFRSSVLHSTQDLFLPFFSNVTWFHVI
SGTNGTKRFDNPVLPFNDGVYFASIEKSNIIRGWIFGTTLDSKTQSLLIVNNATNVVIKVC
EFQFCNDPFLDHKNNKSWMESEFRVYSSANNCTFEYVSQPFLMDLEGKQGNFKNLREF
VFKNIDGYFKIYSKHTPILVRDLPQGFSALEPLVDLPIGINITRFQTLLALHRSYLTPGDSSS
GWTAGAAAYYVGYLQPRTFLLKYNENGTITDAVDCALDPLSETKCTLKSFTVEKGIYQT
SNFRVQPTESIVRFPNITNLCPFDEVFNATRFASVYAWNRKRISNCVADYSVLYNLAPFF
TFKCYGVSPTKLNDLCFTNVYADSFVIRGDEVRQIAPGQTGNIADYNYKLPDDFTGCVI
AWNSNKLDSKVSGNYNYLYRLFRKSNLKPFERDISTEIYQAGNKPCNGVAGENCYFPL
RSYSFRPTYGVGHQPYRVVVLSFELLHAPATVCGPKKSTNLVKNKCVNFNFNGLKGTG
VLTESNKKFLPFQQFGRDIADTTDAVRDPQTLEILDITPCSFGGVSVITPGTNTSNQVAVL
YQGVNCTEVPVAIHADQLTPTWRVYSTGSNVFQTRAGCLIGAEYVNNSYECDIPIGAGI
CASYQGGSGGSSIIAYTMSLGAENSVACSNNSIAIPTNFTISVTTEILPVSMTKTSVDCTM
YICGDSTECSNLLLQYGSFCTQLKRALTGIAVEQDKNTQEVFAQVKQIYKTPPIKYFGGF
NFSQILPDPSKPSKRSPIEDLLFNKVTLADAGFIKQYGDCLGDIAARDLICAQKFKGLTVL
PPLLTDEMIAQYTSALLAGTICSGWTFGAGPALQIPFPMQMAYRENGIGVTQNVLYENQ
KLIANQFNSAIGKIQDSLSSTPSALGKLQDVVNHNAQALNTLVKQLSSKFGAISSVLNDI
FSRLDKPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSN
GTHWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQ

In some embodiments, a VFLIP can be a VFLIP_Omicron_BA.2. An exemplary amino acid sequence of a VFLIP_Omicron_BA.2 is shown in SEQ ID NO: 60 below. Mutations are in BOLD. (SEQ ID NO:60)

QCVNLITRTQSYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIKVC
EFQFCNDPFLDVYYHKNNKSWMESEFRVYSSANNCTFEYVSQPFLMDLEGKQGNFKNL
REFVFKNIDGYFKIYSKHTPINLGRDLPQGFSALEPLVDLPIGINITRFQTLLALHRSYLTP
GDSSSGWTAGAAAYYVGYLQPRTFLLKYNENGTITDAVDCALDPLSETKCTLKSFTVEK
GIYQTSNFRVQPTESIVRFPNITNLCPFDEVFNATRFASVYAWNRKRISNCVADYSVLYN
FAPFFAFKCYGVSPTKLNDLCFTNVYADSFVIRGNEVSQIAPGQTGNIADYNYKLPDDFT
GCVIAWNSNKLDSKVGGNYNYLYRLFRKSNLKPFERDISTEIYQAGNKPCNGVAGFNC
YFPLRSYGFRPTNGVGHQPYRVVVLSFELLHAPATVCGPKKSTNLVKNKCVNFNFNGL
TGTGVLTESNKKFLPFQQFGRDIADTTDAVRDPQTLEILDITPCSFGGVSVITPGTNTSNQ
VAVLYQGVNCTEVPVAIHADQLTPTWRVYSTGSNVFQTRAGCLIGAEYVNNSYECDIPI
GAGICASYQGGSGGSSIIAYTMSLGAENSVACSNNSIAIPTNFTISVTTEILPVSMTKTSV
DCTMYICGDSTECSNLLLQYGSFCTQLKRALTGIAVEQDKNTQEVFAQVKQIYKTPPIKY
FGGFNFSQILPDPSKPSKRSPIEDLLFNKVTLADAGFIKQYGDCLGDIAARDLICAQKENG
LTVLPPLLTDEMIAQYTSALLAGTICSGWTFGAGPALQIPFPMQMAYRENGIGVTQNVL
YENQKLIANQFNSAIGKIQDSLSSTPSALGKLQDVVNHNAQALNTLVKQLSSKFGAISSV
LNDILSRLDKPEAEVQIDRLITGRLQSLQTYVTQQLIRAAEIRASANLAATKMSECVLGQ
SKRVDFCGKGYHLMSFPQSAPHGVVFLHVTYVPAQEKNFTTAPAICHDGKAHFPREGV
FVSNGTHWFVTQRNFYEPQIITTDNTFVSGNCDVVIGIVNNTVYDPLQPELDSFKEELDK
YFKNHTSPDVDLGDISGINASVVNIQKEIDRLNEVAKNLNESLIDLQELGKYEQ

In some embodiments, a VFLIP can be a VFLIP_BA4.Beta. An exemplary amino acid sequence of a VFLIP_BA4.Beta is shown in SEQ ID NO: 61 below. (SEQ ID NO:61)

QCVNLITRTQSYTNSFTRGVYYPDKVFRSSVLHSTQDLFLPFFSNVTWFHAISGTN
GTKRFDNPVLPFNDGVYFASTEKSNIIRGWIFGTTLDSKTQSLLIVNNATNVVIKVCEFQF
CNDPFLDVYYHKNNKSWMESEFRVYSSANNCTFEYVSQPFLMDLEGKQGNFKNLREFV
FKNIDGYFKIYSKHTPINLGREPEDLPQGFSALEPLVDLPIGINITRFQTLLALHRSYLTPG
DSSSGWTAGAAAYYVGYLQPRTFLLKYNENGTITDAVDCALDPLSETKCTLKSFTVEKG
IYQTSNFRVQPTESIVRFPNITNLCPFDEVFNATRFASVYAWNRKRISNCVADYSVLYNF
APFFAFKCYGVSPTKLNDLCFTNVYADSFVIRGNEVSQIAPGQTGNIADYNYKLPDDFTG
CVIAWNSNKLDSKVGGNYNYRYRLFRKSNLKPFERDISTEIYQAGNKPCNGVAGVNCY
FPLQSYGFRPTYGVGHQPYRVVVLSFELLHAPATVCGPKKSTNLVKNKCVNFNFNGLT
GTGVLTESNKKFLPFQQFGRDIADTTDAVRDPQTLEILDITPCSFGGVSVITPGTNTSNQV
AVLYQGVNCTEVPVAIHADQLTPTWRVYSTGSNVFQTRAGCLIGAEYVNNSYECDIPIG
AGICASYQGGSGGSSIIAYTMSLGAENSVACSNNSIAIPTNFTISVTTEILPVSMTKTSVDC
TMYICGDSTECSNLLLQYGSFCTQLKRALTGIAVEQDKNTQEVFAQVKQIYKTPPIKYFG
GFNFSQILPDPSKPSKRSPIEDLLFNKVTLADAGFIKQYGDCLGDIAARDLICAQNFKGLT
VLPPLLTDEMIAQYTSALLAGTICSGWTFGAGPALQIPFPMQMAYRFNGIGVTQNVLYE
NQKLIANQFNSAIGKIQDSLSSTPSALGKLQDVVNHNAQALNTLVKQLSSKFGAISSVLN
DILSRLDKPEAEVQIDRLITGRLQSLQTYVTQQLIRAAEIRASANLAATKMSECVLGQSK
RVDFCGKGYHLMSFPQSAPHGVVFLHVTYVPAQEKNFTTAPAICHDGKAHFPREGVFV
SNGTHWFVTQRNFYEPQIITTDNTFVSGNCDVVIGIVNNTVYDPLQPELDSFKEELDKYF
KNHTSPDVDLGDISGINASVVNIQKEIDRLNEVAKNLNESLIDLQELGKYEQ

In some embodiments, a VFLIP can be a VFLIP_BQ.1.1. An exemplary amino acid sequence of a VFLIP BQ.1.1 is shown in SEQ ID NO: 62 below. (SEQ ID NO: 62)

QCVNLITRTQSYTNSFTRGVYYPDKVFRSSVLHSTQDLFLPFFSNVTWFHAISGTN
GTKRFDNPVLPFNDGVYFASTEKSNIIRGWIFGTTLDSKTQSLLIVNNATNVVIKVCEFQF
CNDPFLDVYYHKNNKSWMESEFRVYSSANNCTFEYVSQPFLMDLEGKQGNFKNLREFV
FKNIDGYFKIYSKHTPINLGREPEDLPQGFSALEPLVDLPIGINITRFQTLLALHRSYLTPG
DSSSGWTAGAAAYYVGYLQPRTFLLKYNENGTITDAVDCALDPLSETKCTLKSFTVEKG
IYQTSNFRVQPTESIVRFPNITNLCPFDEVFNATTFASVYAWNRKRISNCVADYSVLYNF
APFFAFKCYGVSPTKLNDLCFTNVYADSFVIRGNEVSQIAPGQTGNIADYNYKLPDDFTG
CVIAWNSNKLDSTVGGNYNYRYRLFRKSKLKPFERDISTEIYQAGNKPCNGVAGVNCY
FPLQSYGFRPTYGVGHQPYRVVVLSFELLHAPATVCGPKKSTNLVKNKCVNFNFNGLT
GTGVLTESNKKFLPFQQFGRDIADTTDAVRDPQTLEILDITPCSFGGVSVITPGTNTSNQV
AVLYQGVNCTEVPVAIHADQLTPTWRVYSTGSNVFQTRAGCLIGAEYVNNSYECDIPIG
AGICASYQGGSGGSSIIAYTMSLGAENSVACSNNSIAIPTNFTISVTTEILPVSMTKTSVDC
TMYICGDSTECSNLLLQYGSFCTQLKRALTGIAVEQDKNTQEVFAQVKQIYKTPPIKYFG
GFNFSQILPDPSKPSKRSPIEDLLFNKVTLADAGFIKQYGDCLGDIAARDLICAQNFKGLT
VLPPLLTDEMIAQYTSALLAGTICSGWTFGAGPALQIPFPMQMAYRFNGIGVTQNVLYE
NQKLIANQFNSAIGKIQDSLSSTPSALGKLQDVVNHNAQALNTLVKQLSSKFGAISSVLN
DILSRLDKPEAEVQIDRLITGRLQSLQTYVTQQLIRAAEIRASANLAATKMSECVLGQSK
RVDFCGKGYHLMSFPQSAPHGVVFLHVTYVPAQEKNFTTAPAICHDGKAHFPREGVFV
SNGTHWFVTQRNFYEPQIITTDNTFVSGNCDVVIGIVNNTVYDPLQPELDSFKEELDKYF
KNHTSPDVDLGDISGINASVVNIQKEIDRLNEVAKNLNESLIDLQELGKYEQ

In some embodiments, the mutant coronavirus spike protein comprises an amino acid sequence shown in Table 1.

### MODES FOR CARRYING OUT THE DISCLOSURE

Applicant provides herein nanoparticle platforms involve the attachment of at least one protein, peptide, antigen, antibody, or antibody fragment to the surface of a particle, to promote an immune response through enhanced trafficking and recognition by cellular receptors. Protein-based platforms are highly biocompatible, can assemble homogenously, and can be effectively tailored to suit any antigen. Protein nanoparticles injected intravenously have been shown to freely travel through circulatory and lymph vessels with rapid accumulation in the thyroid and spleen, which is advantageous for establishing humoral immunity. Furthermore, protein-based platforms enable antigen attachment through genetic fusion or affinity tags complexes, which allows for a homogenous decoration of antigens on the platform.

Two characteristics of nanoparticle platforms contribute to generating the B-cell IgG response: (1) the attachment of the antigen to a larger scaffold, which improves APC uptake and retention in lymph follicles; and (2) the repetitive array of antigens, which enables efficient binding and activation of multiple B-cell receptors. Particles displaying numerous antigens can then facilitate B-cell activation through efficient crosslinking with multiple Bell cell receptors (BCRs). This evidence has been largely demonstrated in side-by-side experiments of the same antigen delivered as sub-unit ectodomain or bearded on different size self-assembling nanoparticles for viral diseases, such as HIV, influenza, and SARS-CoV-2.

Furthermore, the concept of self-assembling nanoparticles has been applied to create new vaccine technologies. Molecules such as ferritin and heat shock proteins (HSPs) are known to naturally assemble into polyhedral shapes. By genetically attaching an antigenic protein to a self-assembling molecule, such as a protein or polypeptide, various shaped nanoparticles are created. A protein-based nanoparticle that incorporates antigenic proteins in their pre-fusion states can be used as an effective vaccine against these viral agents.

Because this technology is essential for the generation of new vaccine technology, additional molecules that can self-assembly into a higher order structure (e.g., 10-mer, 12-mer, a 20-mer) are needed.

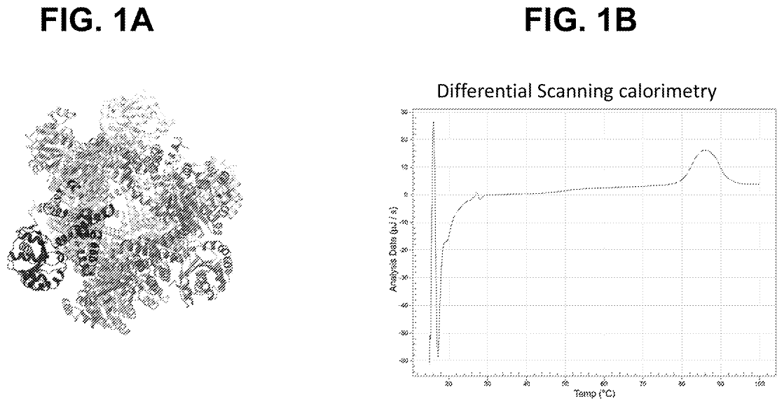

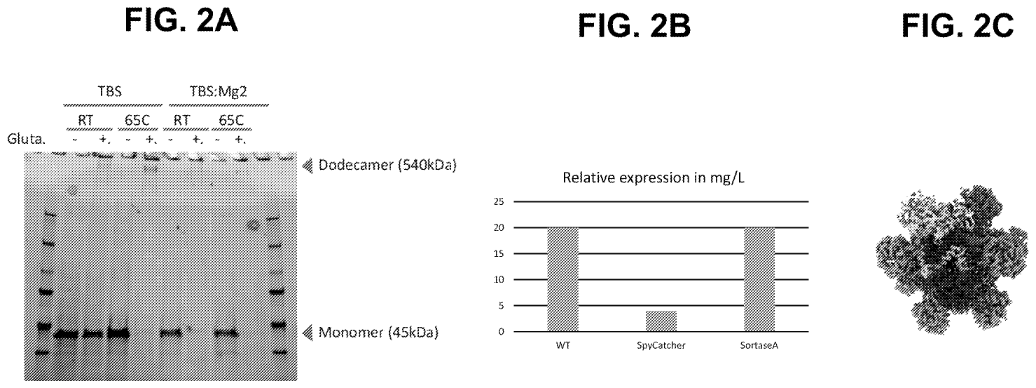

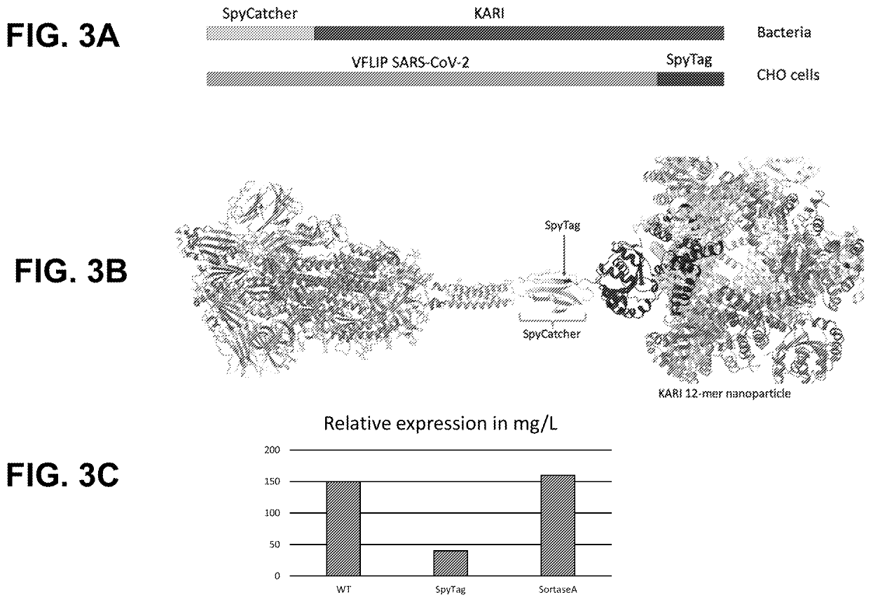

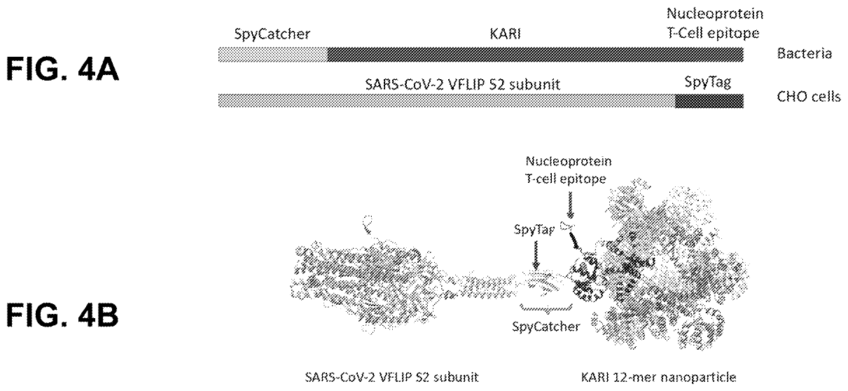

The present disclosure addresses an important considerations in the creation of novel protein-based nanoparticles. In one aspect, the present disclosure provides a self-assembling KARI nanoparticle that can assemble into a dodecamer and as such present up to at least 12 antigenic molecules that can be the same or different on its surface (FIGS. 1, 2C and 3B). Specifically, the present disclosure provides a novel design for a self-assembling multivalent protein nanoparticle as a new platform for antigen displaying and its usage for the development of “universal vaccine candidates.” The novel designs disclosed herein rely on two conjugation systems comprising an archaeal ketol-acid reductoisomerase (KARI) protein and an antigen (FIGS. 3A and 4A). When fused together the recombinant KARI system oligomerizes in a 12-mer nanoparticle with a compact core formed by long helix-alpha that provides a strong stability to the nanoparticle (FIGS. 1, 2C and 3B) that displays the antigen on its surface (FIG. 3B, only showing 1 of the 12 displayed immunogenic molecules).

The binding of the antigen to the KARI nanoparticle was performed by using SpyTag/SpyCatcher system (FIGS. 3A and 4A). The SpyTag/SpyCatcher system uses Streptococcus pyogenes fibronectin-binding protein (FbaB), which has been split in two components: a SpyCatcher component comprising 113 amino acids and SpyTag component comprising 13 amino acids. The nanoparticle platforms that are fused to a SpyCatcher form an irreversible peptide bond with the SpyTag fused to the antigen or vice versa and can be fused to either the N- or C-terminal (FIGS. 3B and 4B). To the best of Applicant's knowledge, the SpyTag/SpyCatcher system is better than other systems used in the art because it facilitates the compartmentalization of the production of the nanoparticle. For example, to generate the nanoparticle of the present disclosure, the SpyCatcher-KARI nanoparticle was expressed and produced in a bacterial cell while the glycoprotein (e.g., VFLIP SARS-CoV-2-SpyTag) was expressed and produced in mammalian cells (FIGS. 3A and 4A). The two components were then covalently fused by allowing the SpyTag/SpyCatcher to irreversibly fuse the two components (FIGS. 3B and 4B, only showing 1 of the 12 displayed immunogenic molecules). A second approach for the coupling between the nanoparticle and the antigens disclosed herein involved the sortase reaction.

To the best of Applicant's knowledge, the construction and use of the novel nanoparticle platform of the present disclosure (e.g., KARI nanoparticle) acts as a self-assembling multivalent protein platform for the delivery of different antigens (e.g., more than one antigen, or a plurality of antigens). The different antigens may each be operably linked to a monomer of the dodecameric KARI particle disclosed herein. Alternatively, each monomer of the dodecameric KARI nanoparticle may operably be linked to a plurality of antigens, which may be the same or different. In that embodiment, the dodecameric KARI nanoparticle may comprise up to twelve different antigens. For example, about 2, about 3, about 4, about 5, about 6, about 7, about 8, about 9, about 10, about 11, and/or about 12 different antigens may operably be linked to the dodecameric KARI particle. Alternatively, each monomer of the dodecameric KARI particle may be operably linked to a plurality of antigens (e.g., one or more antigens). For example, each monomer of the KARI particle may comprise one or more antigens, where the one or more antigens are either the same or different. For example, each monomer may be operably linked to a plurality of antigens. For example, when the plurality of antigens is the same, the KARI nanoparticle displays a single type of antigen. When the plurality of antigens is different, the KARI nanoparticle may display a mixture of antigens.

This property can stimulate and/or enhance an immune response against an antigen tagged to the nanoparticle. Currently, there are a limited number of similar protein nanoparticles available in the art. KARI nanoparticle have competitive properties. The novel nanoparticle platform of the present disclosure is less likely to generate cross-reactive auto-antibodies or reactogenicity in clinical trials because the KARI protein is derived from an archaea, an unicellular organism genetically far from the human being. However, due to its microorganism origin, the novel nanoparticle platform of the present disclosure may trigger an immune response by itself. In that case, a composition comprising the novel nanoparticle platform of the present disclosure may not need an adjuvant to perform the immunizations. As such, the novel nanoparticle platform of the present disclosure is superior to those known in the art for multiple reasons.

First, the cost/effectiveness is superior to other nanoparticle platforms based on the poor yields of the ‘one-gene encoding fused self-assembling nanoparticles. The novel nanoparticle platform of the present disclosure (e.g., KARI nanoparticle) is an easy-expressing costless protein that can be used to deliver multiple antigens, including but not limited to SARS_CoV antigens. The KARI nanoparticle platform can be used for any vaccine candidates against viral or bacterial disease.

Second, the novel nanoparticle platform of the present disclosure allows for the possibility to deliver different antigens (i.e., different SARS-CoV-2 variants) on the same nanoparticle by mixing all the antigens in equimolar concentration. For example, the dodecameric nanoparticle embodiment of the present disclosure allows, for example, the S trimers derived from one or more different coronaviruses or one or more SARS-CoV-2 variants to be displayed on the same dodecameric nanoparticle platforms, with a size ranging from 12.2 to 50.0 nm. Alternatively, the different antigens can each be operably linked to a single monomer of the dodecameric KARI particle. For example, each monomer can be linked to about 2, about 3, about 4, about 5, or more S trimers derived from the same or different coronaviruses or SARS-CoV-2 variants. As such, the KARI nanoparticle can display at least about 12, at least about 24, at least about 36, at least about 48, at least about 60, at least about 72, at least about 84, at least about 96, or at least about 108 antigens, where the antigens are the same or different from each other.

In certain embodiments, each monomer of the dodecameric nanoparticle can operably be linked to a plurality of antigens, which can be the same or different. The dodecameric KARI particle can comprise up to twelve different antigens. For example, about 2, about 3, about 4, about 5, about 6, about 7, about 8, about 9, about 10, about 11, and/or about 12 different antigens are operably linked to the dodecameric KARI particle. Alternatively, each monomer of the dodecameric KARI particle may be operably linked to a plurality (e.g., one or more) antigens, where the antigens are either the same or different. For example, each monomer can be operably linked to a plurality of antigens. For example, each monomer can be operably linked to a plurality of antigens. When the plurality of antigens are the same, the KARI nanoparticle can display a single type of antigen. When the plurality of antigens are different, the KARI nanoparticle can display several types of antigens.

In addition, the use of the high-stability hollow dodecameric nanoparticles disclosed herein allows for the engineering of T-cell epitopes (e.g., PADRE), and peptide adjuvants within the nanoparticle, thus providing, for example, an all-in-one immunogenic composition (e.g., a vaccine solution).

The novel nanoparticle platform of the present disclosure (e.g., KARI) can also be used to display antibodies or nanobodies to improve their avidity. As such, the KARI nanoparticle platform can be useful for cancer immunotherapy. Furthermore, the KARI nanoparticle platform can be useful for identifying and characterizing T-cell epitopes and activity.

Third, the same batch of the nanoparticle produced in bacteria can be used in different vaccines because of the ‘universal’ binding of the SpyTag/SpyCatcher.

The immunogenic polypeptide and composition comprising the immunogenic polypeptides of the present disclosure have several advantageous properties. For example, the protein of interest displayed on the KARI nanoparticle will be presented in its native-like conformation, which enable the protein or proteins to be used as for example vaccine antigens or be displayed multivalently on nanoparticles (e.g., dodecameric nanoparticles). Furthermore, all immunogenic polypeptides or self-assembling KARI nanoparticles can be produced in ExpiCHO cells with high yield. As CHO is one of the principal mammalian cell lines used for industrial manufacture of protein therapeutics and vaccines and ExpiCHO is a transient version of this CHO cell line, KARI nanoparticles obtained from the ExpiCHO production are expected to have the same properties as those from industrial CHO production. Moreover, the high yield of self-assembling nanoparticles produced in ExpiCHO cells will enable the development of a simple, robust, and cost-effective manufacturing process for industrial production. However, the expression of the KARI nanoparticle is not limited to ExpiCHO. The KARI nanoparticle of the present disclosure can also be expressed in any high yield, low cost systems, including for example a baculovirus system.

Finally, Applicant's nanoparticles are useful for a large number of antigens (not only SARS-CoV-2) and the use of various systems (e.g., SpyTag/SpyCatcher, Sortase A) allows to couple and display different antigens, which may be useful for the development of “universal vaccines”. The KARI nanoparticle disclosed herein may be a multivalent nanoparticle that may display at least about 12, at least about 24, at least about 36, at least about 48, at least about 60, at least about 72, at least about 84, at least about 96, or at least about 108 antigens or more, where the antigens are the same or different. When the plurality of antigens are the same, the KARI nanoparticle can display a single type of antigen. When the plurality of antigens are different, the KARI nanoparticle can display several types of antigens.

Accordingly, one aspect of the present disclosure provides a self-assembling nanoparticle comprising, consisting of, consisting essentially of an amino acid sequence of a recombinant Ketol-acid reductoisomerase (KARI; EC 1.1.1.86) and a tether sequence operably linking the KARI to at least one protein or peptide of interest (e.g., an antigen, antibody, or antibody fragment). In some embodiments, each protein comprises a capture sequence.

In another aspect, the present disclosure provides an immunogenic conjugate comprising, consisting of, consisting essentially of: a self-assembling nanoparticle comprising the amino acid sequence of a recombinant Ketol-acid reductoisomerase (KARI; EC 1.1.1.86), and a tether sequence; a protein of interest comprising a capture sequence. In some embodiments, the capture sequence and the tether sequence form a covalent bond that operably links the self-assembling nanoparticle to the protein of interest.

In some embodiments, the recombinant KARI comprises, consists of, consists essentially of the amino acid sequence of SEQ ID NO: 1 or 2, or an amino acid sequence having at least about 37%, at least about 40%, at least about 50%, at least about 55%, at least about 65%, at least about 70%, at least about 75%, at least about 80%, at least about 85%, at least about 90%, at least about 95%, or at least about 99% identity to the amino acid sequence of SEQ ID NO: 1 or 2 across the full length of the amino acid sequence.

In another aspect, the present disclosure provides nucleic acid, expression vectors and compositions comprising, consisting of, consisting essentially of the self-assembling nanoparticle or an immunogenic conjugate comprising, consisting of, consisting essentially of the self-assembling KARI nanoparticle. In another aspect, the present disclosure provides methods of using the self-assembling nanoparticles or the immunogenic conjugate.

### Self-Assembling Nanoparticles

### Keto Acid Reductoisomerase (KARI)

One aspect of the present disclosure provides a self-assembling nanoparticle comprising, consisting of, consisting essentially of an amino acid sequence of a recombinant Ketol-acid reductoisomerase (KARI; EC 1.1.1.86) and a tether sequence operably linking the KARI to at least one protein or peptide of interest (e.g., antigen, antibody, or antibody fragment). In some embodiments, each protein comprises a capture sequence. Acetohydroxy acid isomeroreductase or Ketol-acid reductoisomerase (KARI; EC 1.1.1.86) catalyzes two steps in the biosynthesis of branched-chain amino acids and is a key enzyme in their biosynthesis. For example, Ketol-acid reductoisomerase enzymes are ubiquitous in nature and are involved in the production of valine and isoleucine, pathways that may affect the biological synthesis of isobutanol. Typically, KARI uses NADPH as cofactor and requires a divalent cation such as Mg²⁺ for its activity. In addition to utilizing acetolactate in the valine pathway, KARI also converts acetohydroxybutanoate to dihydroxymethylpentanoate in the isoleucine production pathway.

KARI enzymes are found in a variety of organisms and amino acid sequence comparisons across species. The crystal structure of the E. coli KARI enzyme at 2.6 A resolution (Tyagi, et al., Protein Science, 14, 3089-3100, 2005) showed that E. coli KARI consists of two domains, one with mixed a/s structure which is similar to that found in other pyridine nucleotide-dependent dehydrogenases. The second domain is mainly α-helical and shows strong evidence of internal duplication. Comparison of the active sites of KARI of E. coli, Pseudomonas aeruginosa, and spinach showed that most residues in the active site of the enzyme occupy conserved positions. However, while the E. coli KARI was crystallized as a tetramer, the P. aeruginosa KARI (Ahn, et al., J. Mol. Biol., 328, 505-515, 2003) formed a dodecamer, and the enzyme from spinach formed a dimer. Indeed, only a selected species encodes a KARI gene that can form a dodecamer. See e.g., Lemaire et al, Biomolecules 11: 1679—(2021).

Accordingly, in some embodiments, the recombinant KARI is an archeal, bacterial, or proteobacteria KARI. In some embodiments, the recombinant KARI is selected from Methanothermococcus thermolithotrophicus (MtKARI), Helicobacter pylori, Pseudomonas Aeruginosa (PaKARI), Saccharolobus solfataricus (SacsKARI), Sulfolobus solfataricus (Sso-KARI), A. vinelandii, Sulfolobus sp. E5-1-F, Sulfolobus islandicus, Saccharolobus shibatae, Saccharolobus caldissimus, Saccharolobus shibatae, Stygiolobus sp. KN-1, Sulfolobus sp., Sulfodiicoccus acidiphilus, Acidianus brierleyi, Acidianus manzaensis, Thaumarchaeota archeon, Candidatus Bathyarchaeota, Nitrososphaeria archaeon, Dictyoglomi bacterium, Vulcanisaeta sp, Deltaproteobacteria bacterium, or Acidiplasma cupricumulans.

### KARI Nanoparticles

A self-assembling nanoparticle refers to a ball-shape protein shell with a diameter of tens of nanometers and well-defined surface geometry that is formed by identical copies of a non-viral protein capable of automatically assembling into a nanoparticle. Known examples of self-assembling nanoparticle molecule include ferritin (FR), which is conserved across species, as well as B. stear other mophilus dihydrolipoyl acyltransferase (E2p), Aquifex aeolicus lumazine synthase (LS), and Thermotoga maritima encapsulin. Nanoparticles can form spontaneously upon recombinant expression of the protein in an appropriate expression system. Methods for nanoparticle production, detection, and characterization are known in the art. See e.g., WO2019241483A1.

In general, the KARI nanoparticles are formed by multiple copies of a single KARI recombinant protein subunit. In some embodiments, the recombinant KARI is a dodecameric KARI. In some embodiments, the recombinant KARI is a dodecameric KARI selected from M. thermolithotrophicus (MtKARI), H. pylori, P. Aeruginosa (PaKARI), S. solfataricus (SacsKARI), or S. solfataricus (Sso-KARI). In some embodiments, the dodecameric KARI nanoparticle displays at least about twelve molecules of the protein of interest on the surface of the Dodecameric KARI nanoparticle.

In various embodiments, the employed self-assembling nanoparticles have a diameter of about 25 nm or less (usually assembled from 12, 24, or 60 subunits) and 3-fold axes on the particle surface. Such nanoparticles provide suitable particle platforms to produce multivalent vaccines or universal vaccine platform. In some preferred embodiments, the immunogenic conjugate comprises a self-assembling nanoparticle with a diameter of about 20 nm or less (usually assembled from 12, 24, or 60 subunits) and 3-fold axes on the particle surface. Such nanoparticles provide suitable particle platforms to produce multivalent vaccines. In some embodiments, the self-assembly KARI nanoparticle described herein comprises a diameter of about 26.3 nm to about 37.6 nm.

The KARI nanoparticles disclosed herein can be used as a multivalent system to deliver various proteins or polypeptides of interests including, but not limited to T-cell epitopes. The KARI nanoparticles disclosed herein can also be used to display different molecules of interest (e.g., antigens, antibodies, drugs, adjuvants) via a coupling system (e.g., SpyTag/SpyCatcher system, Sortase A system, etc.) to generate a multivalent system capable of delivering molecules, T-cell epitopes, or any antigenic peptides.

For example, the KARI nanoparticles can be used to display antigens from any source. Accordingly, any T cell epitope from any source can be used with the KARI nanoparticles disclosed herein. The source can be viral, fungal, bacterial, or a tumor antigen. As such, the KARI nanoparticles can be used to display antigens (e.g., SARS-CoV-2 RBDs domains) from different bacterial or viral strains of concern. The immunogenicity of these KARI nanoparticles (e.g., KARI-SARS-CoV-2 RBD) can be evaluated in mice or hamster; and/or can be used to generate a broadly universal vaccine candidate (e.g., SARS-CoV-2 vaccine candidate). In addition, the KARI nanoparticle of the present disclosure can be used to display antigens from one or more sources (e.g., a bacterial, a viral, a fungal, or a tumor antigen) on the same or different monomer. In some embodiments, the KAR nanoparticle can used to display an antigen derived from a bacterium, a virus, a fungus, or a tumor. In some embodiments, the KARI nanoparticle can display short T-cell epitopes or any antigen peptides. In some embodiments, the KARI nanoparticle can display any T cell epitope from any source (e.g., a bacterial, a viral, a fungal, or a tumor antigen).

The KARI nanoparticles can be used to display a plurality of antigens in combination with the VFLIP technology disclosed herein, to measure whether the immunization in a multimeric environment (nanoparticle) can induce a higher and more durable neutralizing antibody response than the soluble spike subunits alone.

In some embodiments, the KARI nanoparticle can be used as a multivalent system to deliver short T-cell epitopes or any antigen peptides. Short peptides elicit poor immune responses and the best T-cell epitopes that could be included on a future vaccine are difficult to map. Accordingly, genetically modified T-cell epitopes or any short antigen peptides can be fused to the KARI nanoparticle to significantly increase the immunogenicity to those epitopes. The use of the nanoparticle disclosed herein is not limited to SARS-CoV-2, as any antigens derived from, for example, a bacterial, a viral, a fungal, or a tumor antigen can be used. Any T cell epitope from any source (e.g., bacterial, viral, fungal, a tumor) can be used with the KARI nanoparticle.

For example, a KARI nanoparticle as described herein can comprise a plurality of antigens, which are the same (i.e., the KARI nanoparticle displays a single type of antigen). In other embodiments, the KARI nanoparticle can comprise a plurality of antigens, which are different (e.g., the KARI nanoparticle can display several types of antigens). The plurality of antigens can each be operably linked to a monomer of the dodecameric KARI particle disclosed herein. For example, one antigen per monomer for a total of 12 antigens on a single dodecameric KARI nanoparticle that can be the same or different.

If the antigens are different, the dodecameric KARI particle can comprise up to twelve different antigens. For example, at least about 2, at least about 3, at least about 4, at least about 5, at least about 6, at least about 7, at least about 8, at least about 9, at least about 10, at least about 11, and/or at least about 12 different antigens can be operably linked to a single dodecameric KARI particle.

In another embodiment, each monomer of the dodecameric KARI nanoparticle can operably be linked to a plurality of antigens, which can be the same or different. For example, each monomer of the dodecameric KARI particle can be operably linked to one or more antigens, where the antigens are either the same or different. For example, each monomer can be linked to 2, 3, 4, 5, 6, 7, 8, 9 or more antigens that can be the same or different. In that embodiment, the KARI nanoparticle can display at least about 24, at least about 36, at least about 48, at least about 60, at least about 72, at least about 84, at least about 96, or at least about 108 or more antigens, where the antigens are the same or different. When the plurality of antigens is the same, the KARI nanoparticle can display a single type of antigens. When the plurality of antigens is different, the KARI nanoparticle can display several types of antigens.

In some embodiments, the KARI nanoparticle comprises 2 antigens per KARI monomer and/or the two antigens are the same or different. In some embodiments, the KARI nanoparticle comprises 3 antigens per KARI monomer and/or the three antigens are the same or different. In some embodiments, the KARI nanoparticle comprises 4 antigens per KARI monomer and/or the four antigens are the same or different. In some embodiments, the KARI nanoparticle comprises 5 antigens per KARI monomer and/or the five antigens are the same or different. In some embodiments, the KARI nanoparticle comprises 6 antigens per KARI monomer and/or the six antigens are the same or different. When the antigens are different, they can be from the same species or different species; or they can target the same epitope or different epitopes on the same receptor and/or cell. In other embodiments, the different antigens can target different receptors on different cell types.

In some embodiments, the recombinant KARI comprises the amino acid sequence of SEQ ID NO: 1 or 2, or an amino acid sequence having at least about 37%, at least about 40%, at least about 50%, at least about 55%, at least about 65%, at least about 70%, at least about 75%, at least about 80%, at least about 85%, at least about 90%, at least about 95%, or at least about 99% identity to the amino acid sequence of SEQ ID NO: 1 or 2.

In some embodiments, the recombinant KARI comprises the amino acid sequence of SEQ ID NO: 2. In some embodiments, the recombinant KARI comprises a deletion in the amino acid sequence of SEQ ID NO: 1. In some embodiments, the recombinant KARI comprises a deletion of about 2, about 3, about 5, about 10, about 12, about 15, or about 20 amino acids in the N-terminus of SEQ ID NO: 1. In some embodiments, the recombinant KARI comprises a deletion of at least 8 amino acids in the N-terminus of SEQ ID NO: 1.

Additionally, or alternatively, the amino-terminus of the particle subunit (e.g., KARI recombinant protein) has to be exposed and in close proximity to the 3-fold axis, and the spacing of three amino-termini has to closely match the spacing of the carboxyl-termini of the displayed protein of interest. In some embodiments, the self-assembling nanoparticle further comprises a detectable label.

In various aspects, the present disclosure provides self-assembling KARI nanoparticles comprising a recombinant KARI protein, as well as their conservatively modified variants or variants with substantially identical (e.g., at least 90%, 95% or 99% identical) amino acid sequences to the KARI protein.

### Tether and Capture Sequences

As used herein, tether and capture sequences are protein or peptide sequences that can form a covalent bond to each other spontaneously or enzymatically with or without addition or involvement of other factors, such as proteins, enzymes, catalysts, salts etc.

Any ligation system known in the art can be used to genetically engineer the self-assembling nanoparticle disclosed herein. In some embodiments, the tether and capture sequence are components of a protein ligation system selected from the SpyTag/SpyCatcher to link the POI to the recombinant KARI such as the SnoopTag/SnoopCatcher (Veggiani et al., Proc Natl Acad Sci USA 113: 1202-1207 (2016)), SpyTag/KTag/SpyLigase (Fierer et al., Proc Natl Acad Sci USA. 111:E1 176-1181 (2014)), Sortase system (Schmohl et al., current Opinion in Chemical Biology. 22: 122-128 (2014)), split inteins (Thiel et al., Angew Chem Int Ed Engl. 53 :1306-1310 (2014)), Butelase 1 (Nguyen et al, at Chem Biol. 10:732-738 (2014)), Peptiligase (Toplak et al, Adv Synth Catal. 358:32140-32147 (2016)), or other similar methods.

In some embodiments, the tether sequence is selected from SpyTag, SpyTag001, SpyTag002, SpyTag003, SpyCatcher, SpyCatcher001, SpyCatcher002, SpyCatcher003, SnoopTag, SnoopCatcher, a sortase recognition domain, a sortase bridging domain, a butelase recognition motif, a C-peptide, a split intein, or a peptiligase substrate. In some embodiments, the tether sequence comprises, consists essentially of, or consists of the amino acid sequence of SEQ ID NO: 4, 8, 13, 15, or 28-39 or an amino acid sequence having at least about 70%, at least about 75%, at least about 80%, at least about 85%, at least about 90%, at least about 95%, at least about 97%, or at least about 99% identity to the amino acid sequence of SEQ ID NO: 4, 8, 13, 15, or 28-39 across the full length of the amino acid sequence.

In some embodiments, the capture sequence is selected from SpyTag, SpyTag001, SpyTag002, SpyTag003, SpyCatcher, SpyCatcher001, SpyCatcher002, SpyCatcher003, SnoopTag, SnoopCatcher, a sortase recognition domain, a sortase bridging domain, a butelase recognition motif, a C-peptide, a split intein, or a peptiligase substrate. In some embodiments, the tether sequence is selected from SpyTag, SpyTag001, SpyTag002, SpyTag003, SpyCatcher, SpyCatcher001, SpyCatcher002, SpyCatcher003, SnoopTag, SnoopCatcher, a sortase recognition domain, a sortase bridging domain, a butelase recognition motif, a C-peptide, a split intein, or a peptiligase substrate. In some embodiments, the capture sequence comprises, consists essentially of, or consists of the amino acid sequence of SEQ ID NO: 4, 8, 13, 15, or 28-39 or an amino acid sequence having at least about 70%, at least about 75%, at least about 80%, at least about 85%, at least about 90%, at least about 95%, at least about 97%, or at least about 99% identity to the amino acid sequence of SEQ ID NO: 4, 8, 13, 15, or 28-39 across the full length of the amino acid sequence, respectively.

### SpyTag/SpyCatcher

SpyTag/SpyCatcher ligation system is a peptide tag/binding partner pair that covalently binds to each other to form an irreversible bond. SpyTag/SpyCatcher ligation system uses spontaneous isopeptide formation in naturally occurring proteins to covalently attach one polypeptide to another. A domain from the Streptococcus pyogenes protein FbaB that contains such an isopeptide bond has been split into two parts. One part, the SpyTag, is a 13 amino acid peptide that contains part of the autocatalytic center. The other part, the SpyCatcher, is a 116 amino acid protein domain containing the other part of the center. It was shown that mixing those two polypeptides restores the autocatalytic center and leads to formation of the isopeptide bond, thereby covalently bind the SpyTag to the SpyCatcher. See e.g., Zakeri et al., PNAS, 109:E690-E697 (2012). Further engineering has led to a shorter version of SpyCatcher with only 84 amino acids as well as an optimized version, SpyTag002 and SpyCatcher002 with accelerated reaction. See e.g., Keeble et al., 2017.

In this respect, SpyTag and SpyCatcher are usually expressed as separate fragments. When a protein comprising a SpyTag domain and one comprising a SpyCatcher domain are combined, the two proteins are capable of covalently reconstituting by isopeptide bond formation. This covalent reaction through an isopeptide bond makes the peptide-protein interaction stable under conditions where non-covalent interactions would rapidly dissociate-over long times (e.g., weeks), at high temperature (to at least 95° C.), at high force, or with harsh chemical treatment (e.g., pH 2-11, organic solvent, detergents or denaturants). Components of the SpyTag/SpyCatcher system and the use of appropriate tags to allow for enzymatic ligation, including, but not limited to the use of the “SpyCatcher” and/or the SpyTag sequence motifs is known to the skilled person. See e.g., U.S. Pat. No. 9,547,003.

In some embodiments, the tether sequence is a SpyCatcher and the capture sequence is SpyTag. In that embodiment, the SpyCatcher comprises, consists of, consists essentially of the amino acid sequence of 4, 36, or 37 and the SpyTag comprises, consists of, consists essentially of the amino acid sequence of 13, 38, or 39.

In some embodiments, the tether sequence comprises, consists of, consists essentially of the amino acid sequence of SEQ ID NO: 4 and the capture sequence comprises the amino acid sequence of SEQ ID NO: 13. In some embodiments, the tether sequence comprises the amino acid sequence of SEQ ID NO: 36 and the capture sequence comprises the amino acid sequence of SEQ ID NO: 38. In some embodiments, the tether sequence comprises the amino acid sequence of SEQ ID NO: 37 and the capture sequence comprises the amino acid sequence of SEQ ID NO: 39.

In some embodiments, the tether sequence is a SpyTag and the capture sequence is SpyCatcher. In that embodiment, the tether sequence comprises the amino acid sequence of SEQ ID NO: 13 and the capture sequence comprises the amino acid sequence of SEQ ID NO: 4. In another embodiment, the tether sequence comprises the amino acid sequence of SEQ ID NO: 38 and the capture sequence comprises the amino acid sequence of SEQ ID NO: 36. In some embodiments, the tether sequence comprises the amino acid sequence of SEQ ID NO: 39 and the capture sequence comprises the amino acid sequence of SEQ ID NO: 37.

In some embodiments, the tether (e.g., Spycatcher or SpyTag) sequence is about 5-50 amino acids in length (e.g., about 10, 20, 30, 40 or 50 amino acids in length) and is derived from an amino acid sequence selected from SEQ ID NOs: 40-43. In some embodiments, the capture (e.g., Spycatcher or SpyTag) sequence is derived from an amino acid sequence selected from SEQ ID NOs: 1, 3, 5 or 6 and can be of any length. The tether sequence and capture sequence may be fused to the protein of interest or the recombinant KARI protein at the N- or C-terminus of such proteins or polypeptides or in an internal loop.

In some embodiments, a spacer sequence (e.g., a glycine/serine rich spacer) may flank the tether sequence or the capture sequence in order to enhance accessibility for reaction. The spacer may further include a site for specific proteolysis (e.g., by Factor X, thrombin, enterokinase or tobacco etch virus NIa protease), allowing specific release from a tether or capture sequence.

Thus, in some embodiments, the tether or capture sequence comprises residues 302-308 of the sequence set out in SEQ ID NO: 40, SEQ ID NO: 38 (MGSSHHHHHHSSGLVPRGSVPTIVMVDAYK RYKGSGESGK), SEQ ID NO: 39 (VPTIVMVDAYKRYKS), or a sequence with at least 50% identity to SEQ ID NO: 38-40, where the tether or capture is less than 50 amino acids in length. In certain embodiments, the tether or capture sequence has at least about 55%, about 60%, about 65%, about 70%, about 75%, about 80%, about 85%, about 90%, or about 95% identity to SEQ ID NO: 40 (across the full length of the amino acids) and is less than 50 amino acids in length. More particularly, the tether or capture sequence may comprise residues 301-308, 300-308, 299-308, 298-308, 297-308, 296-308, 295-308, 294-308, 293-308, 292-308, 291-308 or 290-308 of SEQ ID NO: 40 or a sequence with at least about 50% to 95% identity to residues 302-308 of SEQ ID NO: 38 or 40. Preferably the tether or capture sequence comprises the reactive asparagine of position 303 in SEQ ID NO: 40 (i.e., this residue is preferably unchanged). Further, the tether or capture sequence may be a fragment of SEQ ID NO: 38 or 40. In a preferred embodiment, a tether or capture sequence of less than 50 amino acids is used and comprises amino acids 293-308 of the sequence set forth in SEQ ID NO: 40. In that embodiment, the tether or capture sequence comprises a sequence with at least 50% identity thereto to SEQ ID NO: 40 (across the full length of the amino acid sequence).

The tether or capture sequence are length restricted and comprise less than 50 amino acid residues. In some embodiments, the tether or capture sequences do not comprise the sequence of SEQ ID NO: 1, but only specific fragments thereof, or sequences with at least 50%, at least 75%, at least 80%, at least 85%, at least 90% or at least 95% identity to such specific fragments of SEQ ID NO: 1 (across the full length of the amino acid sequence). In some embodiments, the tether or capture sequence comprises or consists essentially of or consist of SEQ ID NO: 38 or a sequence having at least 50% sequence identity to SEQ ID NO: 38 (across the full length of the amino acid sequence).

In some embodiments, the capture or tether sequence comprises, consists essentially of, or consists of residues 31-291 of the sequence set out in SEQ ID NO: 40, SEQ ID NO: 36 (MSYYHHHHHDYDIPTTENLYFQGAMVTTLSGLSGEQGPSGDMTTEEDSATHIKFSKR DEDGRELAGATMELRDSSGKTISTWISDGHVKDFYLYPGKYTFVETAAPDGY EVATAITFTVNEQGQVTVNGEATKGDAHT), SEQ ID NO: 37 (YFQGAMVTTLSGLSGEQGPSGDMTTEEDSATHIKFSKRDEDGRELAGATMELRDSSGK TISTWISDGHVKDFYLYPGKYTFVETAAPDGYEVATAITFTVNEQGQVTVNGEATKGDA HT) or a sequence with at least 50%, at least 75%, at least 80%, at least 85%, at least 90% or at least 95% identity to residues 32-291 of SEQ ID NO: 40 (across the full length of the amino acid sequence) or SEQ ID NO: 36-37 (across the full length of the amino acid sequence). Specifically, excluded is the complete sequence set out in SEQ ID NO: 1. In some embodiments, the capture or tether sequence should contain the reactive lysine corresponding to position 179 of SEQ ID NO: 40. In that embodiment, the binding partner comprises residues 31-292, 31-293, 31-294, 31-295, 31-296, 31-297, 31-298, 31-299, 31-300, 31-301 or 31-302 of the sequence set forth in SEQ ID NO: 40 or a sequence with at least 70% identity thereto SEQ ID NO: 1 (across the full length of the amino acid sequence).

In some embodiments, a tether or capture sequence may be designed from the major pilin protein using the alternative isopeptide bond in the N-terminus. In some embodiments, a tether or capture sequence may be designed from the FCT-2 pilus major pilin FctA/T1 of Streptococcus pyogenes. Therefore, the tether or capture sequence may be designed or is obtainable from an N-terminal fragment of the isopeptide protein and the remaining, truncated or overlapping protein fragment may constitute the capture sequence. The reactive lysine involved in the isopeptide bond at the N-terminus is found at position 36 of SEQ ID NO: 40 and the reactive asparagine involved in the isopeptide bond is found at position 168 of SEQ ID NO: 40. Thus, in a preferred embodiment the tether or capture sequence comprises the reactive lysine residue in this instance and the tether or capture sequence comprises the reactive asparagine. Particularly, a tether or capture sequence comprises residues 31-40 of the sequence set out in SEQ ID NO: 40 or a sequence with at least 70% identity thereto and is less than 50 amino acids in length. The corresponding capture sequence for the above described tether sequence comprises residues 37-304 of the sequence set out in SEQ ID NO: 40 or has a sequence with at least 70% identity thereto, excluding the sequence of SEQ ID NO: 40. Preferably, the reactive residues in the peptide tag and binding partner are not mutated.

In some embodiments, a tether or capture sequence may be designed from an adhesin Ace protein using the alternative isopeptide bond in the N-terminus. In some embodiments, a tether or capture sequence may be designed from a collagen-binding MSCRAMM adhesin Ace of Enterococcus faecalis. Another tether or capture sequence comprises residues 179-184 or 173-185 of the sequence set out in SEQ ID NO: 41 or has a sequence with at least 50% identity to SEQ ID NO: 41 (across the full length of the amino acid sequence) and is less than 50 amino acids in length. In some embodiments, the capture or tether sequence comprises residues 191-317 or 186-318 of SEQ ID NO: 41 or a sequence having at least 50% identity to SEQ ID NO: 41. Specifically excluded are a tether sequence or a capture sequence having the full length sequence of SEQ ID NO: 41.

In some embodiments, the tether or capture sequence may be designed from a collagen adhesin from Staphylococcus aureus. In that embodiment, the tether or capture sequence comprises fragments of SEQ ID NO: 42 that include the asparagine at position 266 or sequences having at least 50% identity to SEQ ID NO: 42) and the tether or capture sequence comprising fragments of SEQ ID NO: 42 that have at least 50% sequence identity thereto (across the full length of the amino acid sequence) and which comprises the lysine residue at position 149. In some embodiment, the tether or capture sequence does not include the asparagine at position 266. Neither the tether sequence nor the capture sequence comprise the full-length of SEQ ID NO: 42.

In some embodiments, the tether or capture sequence may be designed from a fibronectin binding protein Fbab-B from Streptococcus pyogenes. In some embodiments, the tether or capture sequence comprises a fragment of SEQ ID NO: 43 or a sequence at least 70% identical to SEQ ID NO: 43. In that embodiment, the fragment of SEQ ID NO: 43 includes an aspartic acid residue at position 101. In some embodiments, the capture or tether sequence comprises fragments of SEQ ID NO: 43 that contain the reactive lysine of position 15 or sequences having at least 50% identical thereto SEQ ID NO: 43 (across the full length of the amino acid sequence). In some embodiments, neither the tether sequence nor the capture sequence comprises the full-length of SEQ ID NO: 43.

In some embodiments, the tether or the capture sequence comprises SEQ ID NO: 38 (MGSSHHHHHHSSGLVPRGSVPTIVMVDAYKRYKGSGESGK), SEQ ID NO: 39 (VPTIVMVDAYKRYKS), or a sequence with at least 50% identity to SEQ ID NO: 38 or 39 (across the full length of the amino acid sequence), where the tether or the capture is 15 to 40 or 50 amino acids in length. In certain embodiments, the tether or capture sequence comprises at least about 55%, at least about 60%, at least about 65%, at least about 70%, at least about 75%, at least about 80%, at least about 85%, at least about 90%, or at least about 95% identity to SEQ ID NO: 38 or 39 (across the full length of the amino acid sequence) and is less than 50 amino acids in length. Preferably the tether or capture sequence comprises the reactive aspartic acid of position 8 in SEQ ID NO: 39, i.e., this residue is preferably unchanged.

In some embodiments, the capture or tether sequence comprises SEQ ID NO: 36 or 37 or a sequence having at least 50% identity to SEQ ID NO: 36 or 37 (across the full length of the amino acid sequence), where the tether or capture sequence is 15 to 40 or 50 amino acids in length and contains an aspartic acid of position 8 of SEQ ID NO: 37. In some embodiments, the capture or tether sequence comprises or consists of a sequence comprising at least about 55%, at least about 60%, at least about 65%, at least about 70%, at least about 75%, at least about 80%, at least about 85%, at least about 90%, or at least about 95% identity to SEQ ID NO: 36 (across the full length of the amino acid sequence) (MSYYHHHHHDYDIPTTENLYFQGAMVTTLSGLSGEQGPSGDMTTEEDSATHIKFSKR DEDGRELAGATMELRDSSGKTISTWISDGHVKDFYLYPGKYTFVETAAPDGYEVATAIT FTVNEQGQVTVNGEATKGDAHT) or SEQ ID NO: 37 (YFQGAMVTTLSGLSGEQGPSGDMTTEEDSATHIKFSKRDE DGRELAGATMELRDSSGKTISTWISDGHVKDFYLYPGKYTFVETAAPDGYEVATAITFT VNEQGQVTVNG EATKGDAHT) (across the full length of the amino acid sequence). In some embodiments, the capture or tether sequence comprises an amino acid sequence having at least 50% sequence identity to SEQ ID NO: 26 (across the full length of the amino acid sequence) and/or an amino acid sequence comprising a lysine at position 57 of SEQ ID NO: 36.

Other systems for enzymatic ligation which will be known to the skilled person include the use of butelase, asparaginyl peptidase or sortase as described below.

### Sortase

Another means for tethering a protein of interest to a recombinant KARI protein of the present disclosure comprises the use of sortase enzymes and sortase recognition and bridging domains. The sortase system enables covalent conjugation of polypeptides at specific pre-determined sites. In the sortase system (Schmohl et al., 2014), a short peptide (the sorting motif) is genetically fused to the C-terminus of one polypeptide and two glycine residues are genetically fused to the N-terminus of a second peptide (or vice versa). In the presence of the sortase enzyme, the two modified polypeptides are fused together.

Sortases are transpeptidases produced by Gram-positive bacteria to anchor cell surface proteins covalently to the cell wall. The Staphylococcus aureus sortase A (SrtA) cleaves a short C-terminal recognition motif (LPXTG (SEQ ID NO: 28) (referred to herein as a sortase recognition domain). The sortase recognition domain is a sortase A recognition domain or a sortase B recognition domain. In particular embodiments, the sortase recognition domain comprises or consists of the amino acid sequence: LPTGAA (SEQ ID NO: 29), LPTGGG (SEQ ID NO: 30), LPKTGG (SEQ ID NO: 31), LPETG (SEQ ID NO: 32), LPXTG (SEQ ID NO: 33) or LPXTG(X)ₙ (SEQ ID NO: 34), where X is any amino acid, and n is 0, 1, 2, 3, 4, 5, 7, 8, 9, 10, in the range of 0-5 or 0-10, or any integer up to 100. The sortase recognition domain can be fused, in frame, to a protein of interest or a recombinant KARI protein, optionally through a glycine/serine rich spacer. Where it is attached to a recombinant KARI protein, it is considered a tether sequence and where it is attached to a protein of interest (e.g., antigen), it is considered a capture sequence.

In some embodiments, the tether sequence is a sortase Tag and the capture sequence is sortase A. In some embodiments, the tether sequence is a sortase A and the capture sequence is sortase Tag. In those embodiments, the tether sequence comprises the amino acid sequence of SEQ ID NO: 8 and the capture sequence comprises the amino acid sequence of SEQ ID NO: 15. In those embodiments, the tether sequence comprises the amino acid sequence of SEQ ID NO: 15 and the capture sequence comprises the amino acid sequence of SEQ ID NO: 8. In some embodiments, the sortase recognition motif (i.e., tether or capture sequence) comprises the amino acid sequence of SEQ ID NO: 28-35. In some embodiments, the sortase recognition motif (i.e., tether or capture sequence) is a sortase A recognition motif and comprises, consists of, or consists essentially of the amino acid sequence of SEQ ID NO: 28-34. In some embodiments, the sortase recognition motif (i.e., tether or capture sequence) is a sortase B recognition motif and comprises, consists of, or consists essentially of the amino acid sequence of SEQ ID NO: 35.

The sortase A bridging domain comprises one or more glycine residues at one of its termini. In certain embodiments, the one or more glycine residues may optionally be: Gly, (Gly)2, (Gly)3, (Gly)4 (SEQ ID NO: 63), or (Gly)x (SEQ ID NO: 64), where x is an integer of 1-20. The sortase bridging domain can be attached to a protein of interest or a recombinant KARI protein, optionally through a glycine/serine rich spacer. Where it is attached to a recombinant KARI protein, it is considered a tether sequence and where it is attached to a protein of interest (e.g., an antigen), it is considered a capture sequence.

The sortase B recognition domain comprises the amino acid sequence NPX1TX2, where XI is glutamine or lysine; X2 is asparagine or glycine; N is asparagine; P is proline and T is threonine. The sortase recognition domain can be fused, in frame, to a protein of interest or a recombinant KARI protein, optionally through a glycine/serine rich spacer. Where it is attached to a recombinant KARI protein, it is considered a tether sequence and where it is attached to a protein of interest, it is considered a capture sequence.

The sortase B bridging domain comprises one or more glycine residues at one of its termini. In certain embodiments, the one or more glycine residues may optionally be: Gly, (Gly)2, (Gly)3, (Gly)4 (SEQ ID NO: 63), or (Gly)x (SEQ ID NO: 64), where x is an integer of 1-20. The sortase bridging domain can be attached to a POI or an anchor protein, optionally through a glycine/serine rich spacer. Where it is attached to a recombinant KARI protein, it is considered a tether sequence and where it is attached to a protein of interest, it is considered a capture sequence.

### SpyLigase/SnoopLigase

A further modification of the SpyTag/SpyCatcher system described above resulted in the generation of SpyLigase (Fierer et al., 2014), which was achieved by splitting the FbaB domain into three parts, the SpyTag, the K-tag and the SpyLigase. SpyTag and K-tag are both short peptides that are covalently fused by addition of SpyLigase.

Accordingly, the protein of interest (e.g., an antigen) or a KARI can be attached to exposed surface proteins using the systems described in WO2016/193746. In some embodiments, the tether or capture sequence is attached to the protein of interest or a recombinant KARI protein, optionally through linker sequences, such as a glycine/serine rich spacer. The tether or capture sequence is then ligated by a ligase that is also encoded by the host cell. The tether sequence can, in some embodiments, have a length between 6-50 amino acids, e.g., 7-45, 8-40, 9-35, 10-30, 11-25 amino acids in length, e.g., it may comprise or consist of 6, 7, 8, 9, 10, 11, 12, 13, 14, 15, 16, 17, 18, 19 or 20 amino acids. In some embodiments, the tether or capture sequence comprises about 20-300 amino acids in length. In some embodiments, the tether or capture sequence comprises about 10, about 20, about 30, about 40, about 50, about 60, about 70 amino acids length. In some embodiments, the peptide ligase may be between 50-300 amino acids in length, e.g., 60-250, 70-225, 80-200 amino acids in length, e.g., it may comprise or consist of 60, 65, 70, 75, 80, 85, 90, 95, 100, 110, 120, 130, 140, 150, 160, 170, 180, 190 or 200 amino acids, providing it meets the definitions set forth for the peptide ligase.

As used herein a peptide ligase is an enzyme that forms the isopeptide bond. In some embodiments, the peptide ligase is a fragment of a major pilin protein (SEQ ID NO: 40) comprising a glutamic acid residue at position 117. In that embodiments, the isopeptide bond in the major pilin protein occurs between the lysine residue at position 36 of SEQ ID NO: 40 and the asparagine residue at position 168 of SEQ ID NO: 40. The glutamic acid residue which induces isopeptide formation is found at position 117 in SEQ ID NO: 40. Thus, a pair of tether sequences developed from an isopeptide protein set forth in SEQ ID NO: 40 will preferably comprise a tether sequence comprising a fragment of the protein comprising the reactive asparagine at position 303 and a tether sequence comprising a fragment of the protein comprising the reactive lysine at position 179.

In some embodiments, the peptide ligase is a fragment of Collagen Adhesin (SEQ ID NO: 41) comprising the aspartic acid residue at position 213. In that embodiment, the isopeptide bond occurs between a lysine residue at position 181 of SEQ ID NO: 41 (and an asparagine residue at position 294 of SEQ ID NO: 41. The bond is induced by the aspartic acid residue at position 213 in SEQ ID NO: 41. Thus, a pair of tether sequences developed from an isopeptide protein set forth in SEQ ID NO: 41 will preferably comprise a tether sequence comprising a fragment of the protein comprising the reactive lysine residue at position 36 and a tether sequence comprising a fragment of the protein comprising the reactive asparagine at position 168.

In some embodiments, the peptide ligase is a fragment of a collagen-binding MSCRAMM adhesin Ace (SEQ ID NO: 42) comprising the aspartic acid residue at position 209. In that embodiment, the isopeptide bond occurs between lysine at position 176 of SEQ ID NO: 42 and asparagine at position 308 of SEQ ID NO: 42. The aspartic acid residue that induces the isopeptide bond is at position 209 of SEQ ID NO: 42. Thus, a pair of tether sequences developed from the isopeptide protein set forth in SEQ ID NO: 42 will preferably comprise a tether sequence comprising a fragment of the protein comprising the reactive lysine at position 176 and a tether sequence comprising a fragment of the protein comprising the reactive asparagine at position 308.

In some embodiments, the peptide ligase is a fragment of a fibronectin binding protein Fbab-B (SEQ ID NO: 43) comprising the glutamic acid residue at position 61. In that embodiment, the isopeptide bond in the Fbab-B domain forms between a lysine at position 15 of SEQ ID NO: 43 and an aspartic acid residue at position 101 of SEQ ID NO: 43. The glutamic acid residue which induces the isopeptide bond is at position 61 of SEQ ID NO: 43. Thus, a pair of tether sequences developed from the isopeptide protein set forth in SEQ ID NO: 43 will preferably comprise a tether sequence comprising a fragment of the protein comprising the reactive lysine at position 15 and a tether sequence comprising a fragment of the protein comprising the reactive aspartic acid at position 101.

Additional proteins that can be used to form isopeptide bonds using the SpyLigase ligase system are known to those of skill in the art, such as the RrgA protein is an adhesion protein from Streptococcus pneumonia, or the PsCs protein is a fragment of the por secretion system C-terminal sorting domain protein from Streptococcus intermedius.

### Butelase 1

Another means for tethering a protein of interest to a recombinant KARI protein comprises the use of butelase 1 to form a peptide bond between the butelase recognition motif. Butelase 1 is the enzyme responsible for the macrocyclization of cliotides, cyclotides from C. ternatea, during their biosynthesis and recognizes a linear precursor with a C-terminal tripeptide motif Asn/Asp(Asx)-His-Val. It cleaves the bond between Asx and His to accept an N-terminal residue Xaa, resulting in a new Asx-Xaa bond in the cyclized peptide. Butelase 1 exhibits not only the highest catalytic kinetics among all the peptide ligases found so far, but also a broad substrate specificity for the N-terminal amino acid, Xaa which can be any amino acid except Pro, making it an attractive tool for bioconjugation and peptide ligation (Chem Commun (Camb). 2015, 51, 17289-17292; Angew Chem Int Ed Engl. 2015, 54, 15694-15698; J Am Chem Soc. 2015, 137, 15398-15401).

The butelase recognition motif requires the presence of Asx-X-R at the C-terminus of a first peptide and a P-Asx to the N-terminus of a second peptide, where Asx is Asn or Asp and P is any amino acid. Butelase can be used for enzymatic coupling and has an ability to site-specifically break a peptide bond and then reform a new bond with an incoming nucleophile. Butelase is “Asx-specific” in that the amino acid C-terminal to which ligation occurs, i.e. the C-terminal end of the peptide that is ligated, is either Asn or Asp, preferably Asn. Butelase recognizes the motif Asx-X-R, at the C-terminus of the first peptide, and mediates peptide ligation by cleaving off the sorting signal Asx-X-R and ligating P¹-Asx to the N-terminal residue of the second peptide P¹-Xaa¹-Xaa²-P² to form a ligated peptide P¹-Asx-Xaa¹-Xaa²-P².

In some embodiments, the Asx-His-Val motif can be fused, in frame, to a protein of interest or a recombinant KARI protein, optionally through a glycine/serine rich spacer. See e.g., WO 2017/058114, which is hereby incorporated by reference in its entirety.

### Split Inteins

Another method for tethering a protein of interest to a recombinant KARI comprises the use of split inteins. Inteins can exist as two fragments encoded by two separately transcribed and translated genes. These so-called split inteins self-associate and catalyze protein-splicing activity in trans. Split inteins have been identified in diverse cyanobacteria and archaea (Caspi et al., 2003; Choi J. et al., 2006; Dassa B. et al., 2007; Liu X. and Yang L, 2003; Wu H. et al., 1998; and Zettler J. et al., 2009, the disclosures of which are hereby incorporated by reference in their entireties). A person of skill in the art knows how to use split inteins to fuse heterologous proteins. See e.g., Thiel et al. (2014) and WO 2013/045632.

Purification Tags

Tags used in the practice of the invention may serve any number of purposes and a number of tags may be added to impart one or more different functions to the self-assembling nanoparticle, and/or derivatives thereof, of the disclosure. For example, tags may (1) contribute to protein-protein interactions both internally within a protein and with other protein molecules, (2) make the protein amenable to purification methods, (3) enable one to identify whether the protein is present in a composition; or (4) give the protein other functional characteristics.

In some embodiments, the self-assembling nanoparticle described herein further comprises a tag protein selected from the group consisting of an affinity tag, a fluorescent tag, or an expression and/or solubility enhancement tag. In some embodiments, the tag protein is selected from hexahistidine tag (SEQ ID NO: 65) (his-tag), Fasciola hepatica 8-kDa antigen tag (Fh8), Glutathione-S-transferase (GST) tag, maltose-binding protein tag (MBP), FLAg tag peptide (FLAG tag), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin), small ubiquitin-like modifier tag (SUMO), a strep tag, Thioredoxin (Trx) tag, aVariFlex C-Terminal solubility enhancement tag, a short peptide C-terminal tag, Solubility-enhancer peptide sequences (SET) tag, IgG domain B1 of Protein G (GB1) tag, IgG repeat domain ZZ of Protein A (ZZ) tag, Mutated dehalogenase tag (HaloTag), Solubility eNhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Stress-responsive proteins tag (e.g., RpoA, tag, SlyD Tsf tag, RpoS tag, PotD tag, or Crr tag), and E. coli acidic proteins tag (e.g., msyB tag, yigD tag, and rpoD tag). Additional affinity tags and solubility enhancer tags are known to those skill in the art. See Costa et al., Front. Microbiol., 63(5): (2014); Esposito and Chatterjee Curr. Opin. Biotechnol., 17: 353-358 (2006); Malhotra, A. “Tagging for protein expression,” in Guide to Protein Purification, 2nd Edn, eds. R. R. Burgess and M. P. Deutscher (San Diego, CA: Elsevier), 463:239-258 (2009).

In some embodiments, the tag is selected from hexahistidine tag (SEQ ID NO: 65) (his-tag), small ubiquitin-like modifier tag (SUMO), aVariFlex C-Terminal solubility enhancement tag, a short peptide C-terminal tag, Thioredoxin (Trx) tag, aVariFlex C-Terminal solubility enhancement tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), or fungal avidin-like protein (Tamavidin).

In one embodiment, the tag is an affinity tag selected from a histidine tag such as a hexahistidine tag (SEQ ID NO: 65) (his-tag or 6 His-tag (SEQ ID NO: 65)), Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin). In one embodiment, the tag is a hexahistidine tag (SEQ ID NO: 65).

In some embodiments, the purification tag comprises the amino acid sequence of SEQ ID NO: 6. In some embodiments, the purification tag comprises an amino acid sequence having at least about 70%, at least about 75%, at least about 80%, at least about 85%, at least about 90%, at least about 95%, at least about 97%, or at least about 99% identity to the amino acid sequence of SEQ ID NO: 6 (across the full length of the amino acid sequence).

In some embodiments, the tag is selected from a small ubiquitin-like modifier tag (SUMO), a VariFlex C-Terminal solubility enhancement tag, a short peptide C-terminal tag, Thioredoxin (Trx) tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin).

In some embodiments, the tag further comprises an endoprotein cleavage sequence. In that embodiment, the tag comprises a cleavage sequence recognized by an endoprotein selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase (EnTK), gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin (Thr), tissue kallikrein, tobacco etch virus (TEV), togavirin, tryptophanyl aminopeptidase, U-plasminogen activator, V8, venombin A, venombin AB, factor Xa (Xa), and Xaa-pro aminopeptidase. In some embodiments, the endoprotein cleavage sequence comprises the amino acid sequence of SEQ ID NO: 47, SEQ ID NO: 48, SEQ ID NO: 49, SEQ ID NO: 50 or SEQ ID NO: 51. In some embodiments, the endoprotein cleavage site is selected from ENLYFQ/G (SEQ ID NO: 47), DDDDK/(SEQ ID NO: 48), IEGR/(SEQ ID NO: 49), LVPR/GS (SEQ ID NO: 50), or LEVLFQ/GP (SEQ ID NO: 51).

In some embodiments, the engineered thermostable reverse transcriptase enzyme or a derivative thereof further comprises a protease cleavage sequence. In some embodiments, the cleavage of the protease cleavage sequence by a protease result in cleavage of the affinity tag from the engineered reverse transcriptase enzyme or a derivative thereof. In some instances, the protease cleavage sequence/site is recognized by a protease including, but not limited to, alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase (EnTK), gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin (Thr), tissue kallikrein, tobacco etch virus (TEV), togavirin, tryptophanyl aminopeptidase, U-plasminogen activator, V8, venombin A, venombin AB, factor Xa (Xa), and Xaa-pro aminopeptidase. In some embodiments, the protease cleavage sequence is a thrombin cleavage sequence.

In some embodiment, the tag is cleaved or removed from the engineered thermostablereverse transcriptase enzyme or derivatives thereof via the cleavage site. In one embodiment, the tag is cleaved or removed using an endoprotein selected from the group consisting of tobacco etch virus protease (Tev), enterokinase (EntK), factor Xa (Xa), thrombin (Thr), genetically engineered derivative of human rhinovirus 3C protease (PreScission), Catalytic core of Ulp1 (SUMO protease). In one embodiment, the tag is cleaved at ENLYFQ/G (SEQ ID NO: 47) using tobacco etch virus protease (Tev). In another embodiment, the tag is cleaved at DDDDK/(SEQ ID NO: 48) using Enterokinase (EntK). In another embodiment, the tag is cleaved at IEGR/(SEQ ID NO: 49) using Factor Xa (Xa). In another embodiment, the tag is cleaved at LVPR/GS (SEQ ID NO: 50) using thrombin (Thr). In another embodiment, the tag is cleaved at LEVLFQ/GP (SEQ ID NO: 51) using a genetically engineered derivative of human rhinovirus 3C protease. In another embodiment, the tag is cleaved with Catalytic core of Ulp1 (SUMO protease). Catalytic core of Ulp1 recognizes SUMO tertiary structure and cleaves at the C-terminal end of the conserved Gly-Gly sequence in SUMO.

Protein of interests

A protein of interest” (POI) is any desired polypeptide, peptide, or protein. Non-limiting examples of protein of interest include antibodies, for example full length antibodies, antibody fragments, single chain antibodies (e.g., scFv, scFab), or single domain antibodies, protein scaffolds (e.g., based on fibronectin III, cystatin, lipocalins, Ankyrin repeat domains, Z domain of protein A and others), hormones, interleukins, antigens for the development of vaccines, enzymes, etc. Other examples include, and are not limited to: human growth hormone (hGH), N-methionyl human growth hormone, bovine growth hormone, parathyroid hormone, thyroxine, insulin A-chain, insulin B-chain, proinsulin, relaxin A-chain, relaxin B-chain, prorelaxin, glycoprotein hormones such as follicle stimulating hormones (FSH), thyroid stimulating hormone (TSH), and leutinizing hormone (LH), glycoprotein hormone receptors, calcitonin, glucagon, factor VIII, an antibody, lung surfactant, urokinase, streptokinase, human tissue-type plasminogen activator (t-PA), bombesin, factor IX, thrombin, hemopoietic growth factor, tumor necrosis factor-alpha and -beta, enkephalinase, human serum albumin, mullerian-inhibiting substance, mouse gonadotropin-associated peptide, a microbial protein, such as betalactamase, tissue factor protein, inhibin, activin, vascular endothelial growth factor, receptors for hormones or growth factors; integrin, thrombopoietin, protein A or D, rheumatoid factors, nerve growth factors such as NGF-b, platelet-growth factor, transforming growth factors (TGF) such as TGF-alpha and TGF-beta, insulin-like growth factor-I and -II, insulin-like growth factor binding proteins, CD-4, DNase, latency associated peptide, erythropoietin, osteoinductive factors, interferons such as interferon-alpha, -beta, and -gamma, colony stimulating factors (CSFs) such as M-CSF, GF-CSF, and G-CSF, interleukins (ILs) such as IL-1, IL-2, IL-3, IL-4, superoxide dismutase; decay accelerating factor, viral antigen, HIV envelope proteins such as GP120, GP140, atrial natriuretic peptides A, B or C, immunoglobulins, and fragments of any of the above-listed proteins.

In some embodiments of the self-assembling nanoparticle described herein, the protein of interest is selected from an antigen, a tumor antigen, a viral antigen, a fungal antigen, a bacterial antigen, an antigen for the development of a vaccine, an antibody, an antibody fragment, a single chain antibody, scFv, scFab, single domain antibody, a protein scaffolds, an enzyme, a hormone, or an interleukin.

In some embodiments, the protein of interest is a viral antigen selected from a coronavirus. In some embodiments, the protein of interest is a viral antigen derived from SARS-CoV-1, MERS-CoV, 229E (alpha), NL63 (alpha), OC43 (beta), HKU1 (beta), or SARS-CoV-2. In those embodiments, the protein of interest is a viral antigen derived from a SARS-CoV-2. In that embodiment, the viral antigen is derived from a SARS-CoV-2 variant selected from B.1.1.7, B.1.1.7 with E484K, B.1.135, B.1.351, P.1, B.1.427, D614G, B.1.1351, B.1.429, Lambda (C.37), or Mu (B.1.621).

In some embodiments, the protein of interest is a coronavirus spike protein. In some embodiments, the protein of interest is a mutant coronavirus spike protein. In some embodiments of the self-assembling nanoparticle described herein, the protein of interest is a first-generation “S-2P” spike comprising two proline substitutions at positions 986 and 987 of the spike protein. In some embodiments of the self-assembling nanoparticle described herein, the protein of interest is a second-generation “HexaPro”, comprising four additional prolines at positions 817, 892, 899 and 942 of the Spike protein. In some embodiments of the self-assembling nanoparticle described herein, the protein of interest is a third generation “VFLIP” spike protein.

In some embodiments of the self-assembling nanoparticle described herein, the protein of interest is a mutant coronavirus spike protein comprising 1, 2, 3, 4, or 5 proline mutations selected from F817P, A892P, A899P, A942P, P986K, K986P, V987P, and P987V. In some embodiments, the protein of interest is a mutant coronavirus spike protein comprising at least one additional disulfide bond selected from F43C-G566C, G413C-P987C, Y707C-T883C, G1035C-V1040C, A701C-Q787C, G667C-L864C, V382C-R983C, and 1712C-I816C.

In some embodiments, the mutant coronavirus spike protein comprises the amino acid sequence of SEQ ID NO: 11 or an amino acid sequence having at least about 70%, at least about 75%, at least about 80%, at least about 85%, at least about 90%, at least about 95%, at least about 97%, or at least about 99% identity to the amino acid sequence of SEQ ID NO:11 (across the full length of the amino acid sequence). In some embodiments, the mutant coronavirus spike protein comprises the amino acid sequence disclosed in for example WO 2022/087255A2, which is incorporated by reference in its entirety.

### Additional Structural Components

### Locking Domains

In some embodiments, the self-assembling KARI nanoparticle or immunogenic conjugates of the present disclosure comprises a locking mechanism. The locking mechanism refers to a protein domain (“locking domain”) that functions to stabilize the nanoparticles from the inside in displaying the protein of interest. In general, the locking domain can be any protein capable of forming a dimer. In various embodiments, the locking domain is a protein subunit that can naturally form a dimer with another protein subunit in solution through non-covalent interactions at the interface. In some preferred embodiments, the two protein subunits can be identical in sequence and form a homodimer. In some other cases, the two protein subunits can be different proteins, or two different domains of a single protein derived through engineering, that can form a heterodimer in solution through non-covalent interactions at the interface.

Typically, the locking domain is covalently fused to the nanoparticle subunit to which the protein of interest (e.g., SARS-CoV-2 spike protein) is linked. In some preferred embodiments, the locking domain is selected from dimeric proteins with no more than about 500 amino acids so that it can be encapsulated within a nanoparticle shell. In some embodiments, the locking domain is derived from dimeric proteins with no more than about 400, 300, 250, 200, 150 or fewer amino acids. In some embodiments, the locking domain is derived from dimeric proteins that contains from about 30 to about 100 amino acids. As described herein, the locking domain can be any dimeric protein that can form an interface through specific interactions such as hydrophobic (van der Waals) contacts, hydrogen bonds, and/or salt bridges.

In some embodiments, the locking domain can be any dimeric protein that can form an interface through interactions of helices, sheets, loops, or any combinations of the abovementioned structural elements. In some embodiments, the locking domain can be any dimeric protein that can form an interface at which a covalent bond such as a disulfide bond or a specific chemical linking can be engineered. In various embodiments, the affinity between two subunits of the dimer is sufficiently strong to resist external perturbations such as heat and chemical processing that otherwise would not be tolerated by wild-type (WT) nanoparticles lacking such locking domains. Suitable locking domains can be readily identified from known proteins from Protein database (PDB). For example, dimeric proteins can be found from the protein data bank (PDB) (rcsb.org/) or other databases using keywords such as “homodimer” or search criteria such as “protein stoichiometry A2”. These dimeric proteins can be further filtered based on their size, specifically, 30-100 amino acids.

### Trimerization Domains

Alternative or in additional to the locking domain, the self-assembly nanoparticle described herein can have other structural components. For example, the self-assembling nanoparticle described herein can contain a protein domain that serves to stabilize the protein of interest. In some embodiments, the employed protein domain to achieve this goal can be the C-terminal trimerization motif of T4 fibritin (foldon) that is well known in the art. This foldon domain constitutes the C-terminal 30 amino acid residues of the trimeric protein fibritin from bacteriophage T4, and functions in promoting folding and trimerization of fibritin. See e.g., Papanikolopoulou et al, J. Biol. Chem. 279: 8991-8998, 2004; and Guthe et al., J. Mol. Biol. 337: 905-915, 2004. As exemplified herein with the S spike trimer for SARS-CoV-2 nanoparticle, this protein domain can be readily inserted between S spike subunit and the KARI nanoparticle subunit. In some embodiments, the self-assembling KARI nanoparticle comprises an optional linker (e.g., 10GS linker (SEQ ID NO: 66)) can be used for the insertion. Unlike the locking domain which is inserted at the C-terminus of the KARI nanoparticle subunit, this protein domain (foldon) is inserted at the N-terminus of the KARI nanoparticle subunit. As demonstrated herein with the SARS-CoV-2 nanoparticle, such a structural component (e.g., a foldon) when used alone or in combination with a locking domain, can enhance stability of the protein of interest that is displayed on the surface of the self-assembly nanoparticles.

In some embodiments, the protein of interest is operably linked to a purification tag, and/or a trimerization motif. In some embodiments, the protein of interest is a mutant coronavirus spike protein operably linked to a purification tag, and/or a trimerization motif. In that embodiment, the trimerization motif allows the coronavirus spike to remain trimeric. In that embodiment, the trimerization motif is a T4 fibritin foldon, an HIV-I-derived molecular clamp, or a viral capsid protein SHP. In some embodiment, the trimerization motif comprises the amino acid sequence of SEQ ID NO: 12, 21, or 22 or an amino acid sequence having at least about 70%, at least about 75%, at least about 80%, at least about 85%, at least about 90%, at least about 95%, at least about 97%, or at least about 99% identity to the amino acid sequence of SEQ ID NO: 12, 21, or 22 (across the full length of the amino acid sequence). In some embodiments, the mutant coronavirus spike proteins are formed into dimers, trimers, dodecamers, multimers, or dodecameric nanoparticles.

### T-Cell Epitopes

Adding a T-cell epitope to the C-terminus of the nanoparticle subunit (e.g., the recombinant KARI protein) and/or locking domain notably improved the yield and purity of the resulting KARI nanoparticles. This could be explained by the formation of a hydrophobic T-cell epitope cluster at the center of a hollow KARI nanoparticle. Indeed, KARI nanoparticles comprising a T-cell epitope a showed high-molecular-weight bands on the gel (not shown). Furthermore, KARI nanoparticles comprising a T-cell epitope to the C-terminus showed much stronger binding to neutralizing antibodies when compared to KARI nanoparticle lacking a T-cell epitope.

A T-cell epitope promotes robust T-cell responses and steers B cell development towards the antigen present on the nanoparticle. The T-cell epitope can be located at any position in relation to the other structural components so long as it does not impact presentation of the antigen on the nanoparticle surface. Thus, in some embodiments, the T-cell epitope is located at the C-terminus of the nanoparticle subunit, e.g., by fusing the N-terminus of the T-cell epitope to the C-terminus of the nanoparticle subunit. In some other embodiments, the T-cell epitope is located between C-terminus of the protein of interest (immunogen polypeptide) and N-terminus of the nanoparticle subunit. Any T-cell epitope sequences or peptides known in the art may be employed in the practice of the present invention. They include any polypeptide sequence that contain MHC class-II epitopes and can effectively activate CD4⁺ and CD8⁺ T cells upon immunization, e.g., T-helper epitope that activates CD4+ T helper cells). See e.g., Alexander et al, Immunity 1, 751-761, 1994; Ahlers et al, J. Clin. Invest. 108: 1677-1685, 2001; Fraser et al, Vaccine 32, 2896-2903, 2014; De Groot et al., Immunol. Cell Biol. 8:255-269, 2002; and Gene Ther. 21: 225-232, 2014. In some preferred embodiments, the employed T-helper epitope is the universal pan-reactive T-cell epitope peptide, AKFVAAWTLKAAA (SEQ ID NO: 23) (Alexander et al, Immunity 1, 751-761, 1994). Other examples of suitable T-cell epitopes include peptides QSIALSSLMVAQAIP (SEQ ID NO: 45) and ILMQYIKANSKFIGIPMGLPQSIALSSLMVAQ (SEQ ID NO:46), or conservatively modified variants or substantially identical (e.g., at least 90%, 95% or 99% identical) sequences (across the full length of the amino acid sequence) of any of these exemplified T-cell epitope peptides.

In some embodiments, the self-assembling nanoparticle described herein further comprises a T-cell epitope operably linked to the C-terminus or the N-terminus of the recombinant KARI. In some embodiments, the T-cell epitope is selected from a universal DR epitope (PADRE) T-helper epitope, a CpG-oligodeoxynucleotides (CpG-ODNs), a multi-epitope long peptide, a SARS-CoV-2 nucleo capsid protein N-terminal domain, a SARS-CoV-2 nucleoprotein T-cell epitope. In some embodiments, the T-cell epitope comprises the amino acid sequence of SEQ ID NO: 23-27 or an amino acid sequence having at least about 70%, at least about 75%, at least about 80%, at least about 85%, at least about 90%, at least about 95%, at least about 97%, or at least about 99% identity to the amino acid sequence of SEQ ID NO: 23-27 (across the full length of the amino acid sequence). In some embodiments, the T-cell epitope comprises a SARS-CoV-2 nucleoprotein epitope comprising the amino acid sequence of SEQ ID NO: 26 or 27. In some embodiments, the T-cell epitope is a as a CD4⁺ T-helper epitope or a CD8⁺ T-cell epitope.

### Linkers

In some embodiments of the self-assembling nanoparticle disclosed herein, the self-assembling nanoparticle further comprises a linker. In some embodiments, the linker operably links the recombinant KARI to the tether or capture sequence. In some embodiments, the linker operably links the protein of interest (e.g., an antigen) to the tether or capture sequence. In some embodiments, the linker is a glycine-serine linker or the linker comprises the amino acid sequence of SEQ ID NO: 5, 9, 16-20, 44, or 52-58 or an amino acid sequence having at least about 70%, at least about 75%, at least about 80%, at least about 85%, at least about 90%, at least about 95%, at least about 97%, or at least about 99% identity to the amino acid sequence of SEQ ID NO: 5, 9, 16-20, 44, 52-58 (across the full length of the amino acid sequence).

In some embodiments, the linker is selected from at least one of: GGS, GP, GPGP (SEQ ID NO:54), GGSGGS (SEQ ID NO:55), GGGS (SEQ ID NO:56) or GGGSGGGS (SEQ ID NO:57). In some embodiments, the linker sequence is repeated 1, 2, 3, 4, or 5 times. In still other embodiments, the linker comprises, consists, or consists essentially of: GGGSGGGSGGGSGGGS (SEQ ID NO: 58). In some embodiments, the linker is preferably GGGSGGGSGGGSGGGS (GGGS)4 (SEQ ID NO: 58) or preferably comprises the amino acid sequence of SEQ ID NO: 58.

In various embodiments, self-assembling KARI nanoparticles displaying any of protein of interest (e.g., SARS-CoV-2 spike protein) can be constructed by fusing the protein of interest or subunit of multimeric protein of interest (e.g., a trimer SARS-CoV-2 spike protein spike protein) to the subunit of the nanoparticle (e.g., recombinant KARI protein subunit; SEQ ID NO: 1 or 2) and the locking domain, as well as the other optional or alternative components described herein. To construct the self-assembling KARI nanoparticle or immunogenic conjugate of the present disclosure, one or more linker motifs or moieties may be employed to facilitate connection and maintain structural integrity of the different components. Thus, in some embodiments, a linker motif can be employed to connect the C-terminus of the protein of interest (e.g., SARS-CoV-2 spike protein) to the N-terminus of the recombinant KARI protein (i.e., the nanoparticle subunit). Additionally, or alternatively, a second linker motif can be used to link the C-terminus of the nanoparticle subunit or the C-terminus of the protein of interest to the N-terminus of the locking domain.

In some other embodiments, a third linker motif may be employed to connect the T-cell epitope, e.g., linking the C-terminus of the locking domain to the N-terminus of the T-cell epitope, or linking the C-terminus of the T-cell epitope to the N-terminus of the locking domain. As exemplified herein, linkers can also be used to insert a neck domain or a foldon domain into the nanoparticle constructs. Typically, the linker motifs contain short peptide sequences. In various embodiments, the linkers or linker motifs can be any flexible peptides that connect two protein domains without interfering with their functions. For example, any of these linkers used in the constructs can be GC-rich peptides with a sequence of (GₐSb)n (SEQ ID NO: 67), where “a” is an integer of about 1-5, b is an integer of about 0-2, and n is an integer of about 1-5. In some other embodiments, a T-cell epitope can be used as a linker or part of a linker between the C-terminus of the protein of interest and the N-terminus of the recombinant KARI protein (e.g., nanoparticle subunit).

In some embodiments, a dipeptide linker, GS, can be inserted between the locking domain and the T-cell epitope in any of the self-assembling nanoparticle or immunogenic conjugate of the present disclosure.

TABLE 1
Sequences

| SEQ ID NO | Description | Sequence |
|---|---|---|
| 1 | Wild-type KARI | MDKTVLDANLDPLKGKTIGVIGYGNQGRVQATIMRENGLNVIVGNV |
| | Nanoparticle (WT | KDKYYELAKKEGFEVYEIDEAVRRSDVALLLIPDEVMKEVYEKKIAP |
| | KARI_NP) | VLQGKKEFVLDFASGYNVAFGLIRPPKSVDTIMVAPRMVGEGIMDL |
| | | HKQGKGYPVLLGVKQDASGKAWDYAKAIAKGIGAIPGGIAVISSFEE |
| | | EALLDLMSEHTWVPILFGAIKACYDIAVKEYGVSPEAALLEFYASGE |
| | | LAEIARLIAEEGIFNQMVHHSTTSQYGTLTRMFKYYDVVRRIVENEA |
| | | KYIWDGSFAKEWSLEQQAGYPVFYRLWELATQSEMAKAEKELYKL |
| | | LGRKVKND |
| 2 | KARI Nanoparticle-N- | LKGKTIGVIGYGNQGRVQATIMRENGLNVIVGNVKDKYYELAKKEG |
| | terminal deletion | FEVYEIDEAVRRSDVALLLIPDEVMKEVYEKKIAPVLQGKKEFVLDF |
| | (KARI_NP^{ΔN-Term} | ASGYNVAFGLIRPPKSVDTIMVAPRMVGEGIMDLHKQGKGYPVLLG |
| | | VKQDASGKAWDYAKAIAKGIGAIPGGIAVISSFEEEALLDLMSEHTW |
| | | VPILFGAIKACYDIAVKEYGVSPEAALLEFYASGELAEIARLIAEEGIF |
| | | NQMVHHSTTSQYGTLTRMFKYYDVVRRIVENEAKYIWDGSFAKEW |
| | | SLEQQAGYPVFYRLWELATQSEMAKAEKELYKLLGRKVKND |
| 3 | Spycatcher/KARI_NP | MSATHIKFSKRDEDGKELAGATMELRDSSGKTISTWISDGQVKDFYL |
| | SpyCatcher001-GS | YPGKYTFVETAAPDGYEVATAITFTVNEQGQVTVNGKATKGDAHIG |
| | linker-KARI- | SLKGKTIGVIGYGNQGRVQATIMRENGLNVIVGNVKDKYYELAKKE |
| | Purification tags | GFEVYEIDEAVRRSDVALLLIPDEVMKEVYEKKIAPVLQGKKEFVLD |
| | | FASGYNVAFGLIRPPKSVDTIMVAPRMVGEGIMDLHKQGKGYPVLL |
| | | GVKQDASGKAWDYAKAIAKGIGAIPGGIAVISSFEEEALLDLMSEHT |
| | | WVPILFGAIKACYDIAVKEYGVSPEAALLEFYASGELAEIARLIAEEGI |
| | | FNQMVHHSTTSQYGTLTRMFKYYDVVRRIVENEAKYIWDGSFAKE |
| | | WSLEQQAGYPVFYRLWELATQSEMAKAEKELYKLLGRKVKNDLEV |
| | | LFQGPWSHPQFEKGGGSGGGSGGGSWSHPQFEK* |
| 4 | Spycatcher001 | MSATHIKFSKRDEDGKELAGATMELRDSSGKTISTWISDGQVKDFYL |
| | | YPGKYTFVETAAPDGYEVATAITFTVNEQGQVTVNGKATKGDAHI |
| | GS Linker | GS |
| 6 | Purification tags | LEVLFQGPWSHPQFEKGGGSGGGSGGGSWSHPQFEK |
| 7 | Sortase/KARI_NP | GGGGGSGGSGSLKGKTIGVIGYGNQGRVQATIMRENGLNVIVGNVK |
| | Sortase-GS linker- | DKYYELAKKEGFEVYEIDEAVRRSDVALLLIPDEVMKEVYEKKIAPV |
| | KARI-Purification | LQGKKEFVLDFASGYNVAFGLIRPPKSVDTIMVAPRMVGEGIMDLH |
| | tags | KQGKGYPVLLGVKQDASGKAWDYAKAIAKGIGAIPGGIAVISSFEEE |
| | | ALLDLMSEHTWVPILFGAIKACYDIAVKEYGVSPEAALLEFYASGEL |
| | | AEIARLIAEEGIFNQMVHHSTTSQYGTLTRMFKYYDVVRRIVENEAK |
| | | YIWDGSFAKEWSLEQQAGYPVFYRLWELATQSEMAKAEKELYKLL |
| | | GRKVKNDLEVLFQGPWSHPQFEKGGGSGGGSGGGSWSHPQFEK* |
| 8 | Sortase | GGGGG |
| 9 | GS linker | SGGSGS |
| 10 | VFLIP_SpyTag001 | QCVNLTTRTQLPPAYTNSFTRGVYYPDKVFRSSVLHSTQDLFLPFFSN |
| | VFLIP-Trimerization | VTWFHAIHVSGTNGTKRFDNPVLPFNDGVYFASTEKSNIIRGWIFGTT |
| | Domain-SpyTag001- | LDSKTQSLLIVNNATNVVIKVCEFQFCNDPFLGVYYHKNNKSWMES |
| | Purification tags | EFRVYSSANNCTFEYVSQPFLMDLEGKQGNFKNLREFVFKNIDGYFK |
| | | IYSKHTPINLVRDLPQGFSALEPLVDLPIGINITRFQTLLALHRSYLTPG |
| | | DSSSGWTAGAAAYYVGYLQPRTFLLKYNENGTITDAVDCALDPLSE |
| | | TKCTLKSFTVEKGIYQTSNFRVQPTESIVRFPNITNLCPFGEVFNATRF |
| | | ASVYAWNRKRISNCVADYSVLYNSASFSTFKCYGVSPTKLNDLCFTN |
| | | VYADSFVIRGDEVRQIAPGQTGKIADYNYKLPDDFTGCVIAWNSNNL |
| | | DSKVGGNYNYLYRLFRKSNLKPFERDISTEIYQAGSTPCNGVEGFNC |
| | | YFPLQSYGFQPTNGVGYQPYRVVVLSFELLHAPATVCGPKKSTNLVK |
| | | NKCVNFNFNGLTGTGVLTESNKKFLPFQQFGRDIADTTDAVRDPQTL |
| | | EILDITPCSFGGVSVITPGTNTSNQVAVLYQGVNCTEVPVAIHADQLT |
| | | PTWRVYSTGSNVFQTRAGCLIGAEHVNNSYECDIPIGAGICASYQGG |
| | | SGGSSIIAYTMSLGAENSVACSNNSIAIPTNFTISVTTEILPVSMTKTSV |
| | | DCTMYICGDSTECSNLLLQYGSFCTQLNRALTGIAVEQDKNTQEVFA |
| | | QVKQIYKTPPIKDFGGFNFSQILPDPSKPSKRSPIEDLLFNKVTLADAG |
| | | FIKQYGDCLGDIAARDLICAQKFNGLTVLPPLLTDEMIAQYTSALLAG |
| | | TICSGWTFGAGPALQIPFPMQMAYRFNGIGVTQNVLYENQKLIANQF |
| | | NSAIGKIQDSLSSTPSALGKLQDVVNQNAQALNTLVKQLSSNFGAISS |
| | | VLNDILSRLDKPEAEVQIDRLITGRLQSLQTYVTQQLIRAAEIRASANL |
| | | AATKMSECVLGQSKRVDFCGKGYHLMSFPQSAPHGVVFLHVTYVP |
| | | AQEKNFTTAPAICHDGKAHFPREGVFVSNGTHWFVTQRNFYEPQIIT |
| | | TDNTFVSGNCDVVIGIVNNTVYDPLQPELDSFKEELDKYFKNHTSPD |
| | | VDLGDISGINASVVNIQKEIDRLNEVAKNLNESLIDLQELGKYEQYIK |
| | | GSGYIPEAPRDGQAYVRKDGEWVLLSTFLAHIVMVDAYKPTKLEVL |
| | | FQGPAGWSHPQFEKGGGSGGGSGGGSWSHPQFEK |
| 11 | VFLIP | QCVNLTTRTQLPPAYTNSFTRGVYYPDKVFRSSVLHSTQDLFLPFFSN |
| | | VTWFHAIHVSGTNGTKRFDNPVLPFNDGVYFASTEKSNIIRGWIFGTT |
| | | LDSKTQSLLIVNNATNVVIKVCEFQFCNDPFLGVYYHKNNKSWMES |
| | | EFRVYSSANNCTFEYVSQPFLMDLEGKQGNFKNLREFVFKNIDGYFK |
| | | IYSKHTPINLVRDLPQGFSALEPLVDLPIGINITRFQTLLALHRSYLTPG |
| | | DSSSGWTAGAAAYYVGYLQPRTFLLKYNENGTITDAVDCALDPLSE |
| | | TKCTLKSFTVEKGIYQTSNFRVQPTESIVRFPNITNLCPFGEVENATRF |
| | | ASVYAWNRKRISNCVADYSVLYNSASFSTFKCYGVSPTKLNDLCFTN |
| | | VYADSFVIRGDEVRQIAPGQTGKIADYNYKLPDDFTGCVIAWNSNNL |
| | | DSKVGGNYNYLYRLFRKSNLKPFERDISTEIYQAGSTPCNGVEGFNC |
| | | YFPLQSYGFQPTNGVGYQPYRVVVLSFELLHAPATVCGPKKSTNLVK |
| | | NKCVNFNFNGLTGTGVLTESNKKFLPFQQFGRDIADTTDAVRDPQTL |
| | | EILDITPCSFGGVSVITPGTNTSNQVAVLYQGVNCTEVPVAIHADQLT |
| | | PTWRVYSTGSNVFQTRAGCLIGAEHVNNSYECDIPIGAGICASYQGG |
| | | SGGSSIIAYTMSLGAENSVACSNNSIAIPTNFTISVTTEILPVSMTKTSV |
| | | DCTMYICGDSTECSNLLLQYGSFCTQLNRALTGIAVEQDKNTQEVFA |
| | | QVKQIYKTPPIKDFGGFNFSQILPDPSKPSKRSPIEDLLFNKVTLADAG |
| | | FIKQYGDCLGDIAARDLICAQKFNGLTVLPPLLTDEMIAQYTSALLAG |
| | | TICSGWTFGAGPALQIPFPMQMAYRFNGIGVTQNVLYENQKLIANQF |
| | | NSAIGKIQDSLSSTPSALGKLQDVVNQNAQALNTLVKQLSSNFGAISS |
| | | VLNDILSRLDKPEAEVQIDRLITGRLQSLQTYVTQQLIRAAEIRASANL |
| | | AATKMSECVLGQSKRVDFCGKGYHLMSFPQSAPHGVVFLHVTYVP |
| | | AQEKNFTTAPAICHDGKAHFPREGVFVSNGTHWFVTQRNFYEPQIIT |
| | | TDNTFVSGNCDVVIGIVNNTVYDPLQPELDSFKEELDKYFKNHTSPD |
| | | VDLGDISGINASVVNIQKEIDRLNEVAKNLNESLIDLQELGKYEQYIK |
| | | GSG |
| 12 | Trimerization Domain | YIPEAPRDGQAYVRKDGEWVLLSTFL |
| | Foldron | |
| 13 | SpyTag001 | AHIVMVDAYKPTK |
| 14 | VFLIP_SortaseTag | QCVNLTTRTQLPPAYTNSFTRGVYYPDKVFRSSVLHSTQDLFLPFFSN |
| | VFLIP-Trimerization | VTWFHAIHVSGTNGTKRFDNPVLPFNDGVYFASTEKSNIIRGWIFGTT |
| | Domain-SortaseA- | LDSKTQSLLIVNNATNVVIKVCEFQFCNDPFLGVYYHKNNKSWMES |
| | Purification tags | EFRVYSSANNCTFEYVSQPFLMDLEGKQGNFKNLREFVFKNIDGYFK |
| | | IYSKHTPINLVRDLPQGFSALEPLVDLPIGINITRFQTLLALHRSYLTPG |
| | | DSSSGWTAGAAAYYVGYLQPRTFLLKYNENGTITDAVDCALDPLSE |
| | | TKCTLKSFTVEKGIYQTSNFRVQPTESIVRFPNITNLCPFGEVFNATRF |
| | | ASVYAWNRKRISNCVADYSVLYNSASFSTFKCYGVSPTKLNDLCFTN |
| | | VYADSFVIRGDEVRQIAPGQTGKIADYNYKLPDDFTGCVIAWNSNNL |
| | | DSKVGGNYNYLYRLFRKSNLKPFERDISTEIYQAGSTPCNGVEGFNC |
| | | YFPLQSYGFQPTNGVGYQPYRVVVLSFELLHAPATVCGPKKSTNLVK |
| | | NKCVNFNFNGLTGTGVLTESNKKFLPFQQFGRDIADTTDAVRDPQTL |
| | | EILDITPCSFGGVSVITPGTNTSNQVAVLYQGVNCTEVPVAIHADQLT |
| | | PTWRVYSTGSNVFQTRAGCLIGAEHVNNSYECDIPIGAGICASYQGG |
| | | SGGSSIIAYTMSLGAENSVACSNNSIAIPTNFTISVTTEILPVSMTKTSV |
| | | DCTMYICGDSTECSNLLLQYGSFCTQLNRALTGIAVEQDKNTQEVFA |
| | | QVKQIYKTPPIKDFGGFNFSQILPDPSKPSKRSPIEDLLFNKVTLADAG |
| | | FIKQYGDCLGDIAARDLICAQKFNGLTVLPPLLTDEMIAQYTSALLAG |
| | | TICSGWTFGAGPALQIPFPMQMAYRFNGIGVTQNVLYENQKLIANQF |
| | | NSAIGKIQDSLSSTPSALGKLQDVVNQNAQALNTLVKQLSSNFGAISS |
| | | VLNDILSRLDKPEAEVQIDRLITGRLQSLQTYVTQQLIRAAEIRASANL |
| | | AATKMSECVLGQSKRVDFCGKGYHLMSFPQSAPHGVVFLHVTYVP |
| | | AQEKNFTTAPAICHDGKAHFPREGVFVSNGTHWFVTQRNFYEPQIIT |
| | | TDNTFVSGNCDVVIGIVNNTVYDPLQPELDSFKEELDKYFKNHTSPD |
| | | VDLGDISGINASVVNIQKEIDRLNEVAKNLNESLIDLQELGKYEQYIK |
| | | GSGYIPEAPRDGQAYVRKDGEWVLLSTFLGGSGGSLPETGGGSGGSL |
| | | EVLFQGPAGWSHPQFEKGGGSGGGSGGGSWSHPQFEK |
| 15 | SortaseA/linker | GGSGGSLPETGGGSGGS |
| 16 | linker | GGSGGS |
| | linker | GGS |
| | linker | GP |
| 19 | linker | GPGP |
| 20 | linker | GGGSGGGS |
| 22 | viral capsid protein | EVRIFAGNDPAHTATGSSGISSPTPALTPLMLDEATGKLVVWDGQKA |
| | SHP | GSAVGILVLPLEGTETALTYYKSGTFATEAIHWPESVDEHKKANAFA |
| | | GSALSHAA |
| 23 | an DR epitope | AKFVAAWTLKAAA |
| | (PADRE) T-helper | |
| | epitope | |
| 24 | CpG- | TCCATGACGTTCCTGACGTT |
| | oligodeoxynucleotides | |
| | (CpG-ODNs) | |
| 25 | multi-epitope long | ELAAWCRWGFLLALLPPGIAGRRIRGRILHDGAYSLTLQGLGIH |
| | peptide | |
| 26 | SARS-CoV-2 | LALLLLDRL |
| | nucleoprotein | |
| | epitope-N₂₁₉₋₂₂₇ | |
| 27 | SARS-CoV-2 | LLLDRLNQL |
| | nucleoprotein | |
| | epitope-N₂₂₂₋₂₃₀ | |
| 28 | sortase A recognition | LPXTG |
| | domain | |
| 29 | sortase A recognition | LPTGAA |
| | domain | |
| 30 | sortase A recognition | LPTGGG |
| | domain | |
| 31 | sortase A recognition | LPKTGG |
| | domain | |
| 32 | sortase A recognition | LPETG |
| | domain | |
| 33 | sortase A recognition | LPXTG |
| | domain | |
| 34 | Sortase A recognition | LPXTG(X)ₙ |
| | domain | |
| | Sortase B recognition | NPX1TX2 |
| | domain | |
| 36 | Spycatcher | MSYYHHHHHHDYDIPTTENLYFQGAMVTTLSGLSGEQGPSGDMTTE |
| | | EDSATHIKFSKRDE |
| | | DGRELAGATMELRDSSGKTISTWISDGHVKDFYLYPGKYTFVETAAP |
| | | DGYEVATAITFTV |
| | | NEQGQVTVNGEATKGDAHT |
| 37 | Spycatcher | YFQGAMVTTLSGLSGEQGPSGDMTTEEDSATHIKFSKRDEDGRELAG |
| | | ATMELRDSSGKTI |
| | | STWISDGHVKDFYLYPGKYTFVETAAPDGYEVATAITFTVNEQGQVT |
| | | VNGEATKGDAHT |
| 38 | SpyTag | MGSSHHHHHHSSGLVPRGSVPTIVMVDAYKRYKGSGESGK |
| 39 | SpyTag | VPTIVMVDAYKRYKS |
| 40 | FCT-2 pilus major | MKLRHLLLTGAALTSFAATTVHGETVVNGAKLTVTKNLDLVNSNAL |
| | pilin FctA/T1 | IPNTDFTFKIEPDTTVNEDGNKFKGVALNTPMTKVTYTNSDKGGSNT |
| | [Streptococcus | KTAEFDFSEVTFEKPGVYYYKVTEEKIDKVPGVSYDTTSYTVQVHVL |
| | pyogenes] | WNEEQQKPVATYIVGYKEGSKVPIQFKNSLDSTTLTVKKKVSGTGG |
| | | DRSKDFNFGLTLKANQYYKASEKVMIEKTTKGGQAPVQTEASIDQL |
| | | YHFTLKDGESIKVTNLPVGVDYVVTEDDYKSEKYTTNVEVSPQDGA |
| | | VKNIAGNSTEQETSTDKDMTITFTNKKDFEVPTGVAMTVAPYIALGI |
| | | VAVGGALYFVKKKNA |
| 41 | collagen-binding | MTKSVKFLVLLLVMILPIAGALLIGPISFGAELSKSSIVDKVELDHTTL |
| | MSCRAMM adhesin | YQGEMTSIKVSFSDKENQKIKPGDTITLTLPDALVGMTENDSSPRKIN |
| | Ace [Enterococcus | LNGLGEVFIYKDHVVATFNEKVESLHNVNGHFSFGIKTLITNSSQPNV |
| | faecalis] | IETDFGTATATQRLTIEGVTNTETGQIERDYPFFYKVGDLAGESNQVR |
| | | WFLNVNLNKSDVTEDISIADRQGSGQQLNKESFTFDIVNDKETKYISL |
| | | AEFEQQGYGKIDFVTDNDFNLRFYRDKARFTSFIVRYTSTITEAGQHQ |
| | | ATFENSYDINY |
| | | QLNNQDATNEKNTSQVKNVFVEGEASGNQNVEMPTEESLDIPLETID |
| | | EWEPKTPTSEQATETSEKTDTTETAESSQPEVHVSPTEEENPDEGETL |
| | | GTIEPIIPEKPSVTTEENGTTETAESSQPEVHVSPTEEENPDESETLGTIE |
| | | PIIPEKPSVTTEENGTTETAESSQPEVHVSPAEEENPDESETLGTILPILP |
| | | EKPSVTTEENGTTETAESSQPEVHVSPTEEENPDESETLGTIAPIIPEKP |
| | | SVTTEENGITETAESSQPEVHVSPTKEITTTEKKQPSTETTVEKNKNVT |
| | | SKNQPQILNAPLNTLKNEGSPQLAPQLLSEPIQKLNEANGQRELPKTG |
| | | TTKTPFMLIAGILASTFAVLGVSYLQIRKN |
| 42 | Collagen Adhesin | GSARDISSTNVTDLTVSPSKIEDGGKTTVKMTFDDKNGKIQNGDMIK |
| | [Staphylococcus | VAWPTSGTVKIEGYSKTVPLTVKGEQVGQAVITPDGATITFNDKVEK |
| | aureus] | LSDVSGFAEFEVQGRNLTQTNTSDDK |
| | | VATITSGNKSTNVTVHKSEAGTSSVFYYKTGDMLPEDTTHVRWFLNI |
| | | NNEKSYVSKDITIKDQIQGGQQLDLSTLNINVTGTHSNYYSGQSAITD |
| | | FEKAFPGSKITVDNTKNTIDVTIPQGYGSYNSFSINYKTKITNEQQKEF |
| | | VNNSQAWYQEHGKEEVNGKSFNHTVHNINANAGIEGTVK |
| 43 | fibronectin binding | MTIEEDSATHIKFSKRDIDGKELAGATMELRDSSGKTISTWISDGQVK |
| | protein Fbab-B | DFYLMPGKYTFVETAAPDGYEVATAITFTVNEQGQVTVNGKATKGD |
| | [Streptococcus | AHIVMVDA |
| | pyogenes] | |
| 44 | linker | GGGGSGGGGS |
| 45 | T-cell epitope | QSIALSSLMVAQAIP |
| 46 | T-cell epitope | ILMQYIKANSKFIGIPMGLPQSIALSSLMVAQ |
| 47 | Protease cleavage site | ENLYFQ/G |
| 48 | Protease cleavage site | DDDDK/ |
| 49 | Protease cleavage site | IEGR/ |
| 50 | Protease cleavage site | LVPR/GS |
| 51 | Protease cleavage site | LEVLFQ/GP |
| | Linker | GGS |
| | Linker | GP |
| 54 | Linker | GPGP |
| 55 | Linker | GGSGGS |
| 56 | Linker | GGGS |
| 57 | Linker | GGGSGGGS |
| 58 | Linker | GGGSGGGSGGGSGGGS |
| 59 | VFLIP_Omicron | QCVNLTTRTQLPPAYTNSFTRGVYYPDKVFRSSVLHSTQDLFLPFFSN |
| | (Omicron (B.1.1.529)) | VTWFHVISGTNGTKRFDNPVLPFNDGVYFASIEKSNIIRGWIFGTTLD |
| | | SKTQSLLIVNNATNVVIKVCEFQFCNDPFLDHKNNKSWMESEFRVYS |
| | | SANNCTFEYVSQPFLMDLEGKQGNFKNLREFVFKNIDGYFKIYSKHT |
| | | PILVRDLPQGFSALEPLVDLPIGINITRFQTLLALHRSYLTPGDSSSGW |
| | | TAGAAAYYVGYLQPRTFLLKYNENGTITDAVDCALDPLSETKCTLKS |
| | | FTVEKGIYQTSNFRVQPTESIVRFPNITNLCPFDEVFNATRFASVYAW |
| | | NRKRISNCVADYSVLYNLAPFFTFKCYGVSPTKLNDLCFTNVYADSF |
| | | VIRGDEVRQIAPGQTGNIADYNYKLPDDFTGCVIAWNSNKLDSKVSG |
| | | NYNYLYRLFRKSNLKPFERDISTEIYQAGNKPCNGVAGFNCYFPLRS |
| | | YSFRPTYGVGHQPYRVVVLSFELLHAPATVCGPKKSTNLVKNKCVN |
| | | FNFNGLKGTGVLTESNKKFLPFQQFGRDIADTTDAVRDPQTLEILDIT |
| | | PCSFGGVSVITPGTNTSNQVAVLYQGVNCTEVPVAIHADQLTPTWRV |
| | | YSTGSNVFQTRAGCLIGAEYVNNSYECDIPIGAGICASYQGGSGGSSII |
| | | AYTMSLGAENSVACSNNSIAIPTNFTISVTTEILPVSMTKTSVDCTMYI |
| | | CGDSTECSNLLLQYGSFCTQLKRALTGIAVEQDKNTQEVFAQVKQIY |
| | | KTPPIKYFGGFNFSQILPDPSKPSKRSPIEDLLFNKVTLADAGFIKQYG |
| | | DCLGDIAARDLICAQKFKGLTVLPPLLTDEMIAQYTSALLAGTICSG |
| | | WTFGAGPALQIPFPMQMAYRFNGIGVTQNVLYENQKLIANQFNSAIG |
| | | KIQDSLSSTPSALGKLQDVVNHNAQALNTLVKQLSSKFGAISSVLNDI |
| | | FSRLDKPEAEVQIDRLITGRLQSLQTYVTQQLIRAAEIRASANLAATK |
| | | MSECVLGQSKRVDFCGKGYHLMSFPQSAPHGVVFLHVTYVPAQEK |
| | | NFTTAPAICHDGKAHFPREGVFVSNGTHWFVTQRNFYEPQIITTDNTF |
| | | VSGNCDVVIGIVNNTVYDPLQPELDSFKEELDKYFKNHTSPDVDLGD |
| | | ISGINASVVNIQKEIDRLNEVAKNLNESLIDLQELGKYEQ |
| 60 | VFLIP_Omicron BA.2 | QCVNLITRTQSYTNSFTRGVYYPDKVFRSSVLHSTQDLFLPFFSNVT |
| | mutations | WFHAIHVSGTNGTKRFDNPVLPFNDGVYFASTEKSNIIRGWIFGTTLD |
| | | SKTQSLLIVNNATNVVIKVCEFQFCNDPFLDVYYHKNNKSWMESEFR |
| | | VYSSANNCTFEYVSQPFLMDLEGKQGNFKNLREFVFKNIDGYFKIYS |
| | | KHTPINLGRDLPQGFSALEPLVDLPIGINITRFQTLLALHRSYLTPGDS |
| | | SSGWTAGAAAYYVGYLQPRTFLLKYNENGTITDAVDCALDPLSETK |
| | | CTLKSFTVEKGIYQTSNFRVQPTESIVRFPNITNLCPFDEVFNATRFAS |
| | | VYAWNRKRISNCVADYSVLYNFAPFFAFKCYGVSPTKLNDLCFTNV |
| | | YADSFVIRGNEVSQIAPGQTGNIADYNYKLPDDFTGCVIAWNSNKLD |
| | | SKVGGNYNYLYRLFRKSNLKPFERDISTEIYQAGNKPCNGVAGENCY |
| | | FPLRSYGFRPTNGVGHQPYRVVVLSFELLHAPATVCGPKKSTNLVK |
| | | NKCVNFNFNGLTGTGVLTESNKKFLPFQQFGRDIADTTDAVRDPQTL |
| | | EILDITPCSFGGVSVITPGTNTSNQVAVLYQGVNCTEVPVAIHADQLT |
| | | PTWRVYSTGSNVFQTRAGCLIGAEYVNNSYECDIPIGAGICASYQGG |
| | | SGGSSIIAYTMSLGAENSVACSNNSIAIPTNFTISVTTEILPVSMTKTSV |
| | | DCTMYICGDSTECSNLLLQYGSFCTQLKRALTGIAVEQDKNTQEVFA |
| | | QVKQIYKTPPIKYFGGFNFSQILPDPSKPSKRSPIEDLLFNKVTLADAG |
| | | FIKQYGDCLGDIAARDLICAQKFNGLTVLPPLLTDEMIAQYTSALLAG |
| | | TICSGWTFGAGPALQIPFPMQMAYRFNGIGVTQNVLYENQKLIANQF |
| | | NSAIGKIQDSLSSTPSALGKLQDVVNHNAQALNTLVKQLSSKFGAISS |
| | | VLNDILSRLDKPEAEVQIDRLITGRLQSLQTYVTQQLIRAAEIRASANL |
| | | AATKMSECVLGQSKRVDFCGKGYHLMSFPQSAPHGVVFLHVTYVP |
| | | AQEKNFTTAPAICHDGKAHFPREGVFVSNGTHWFVTQRNFYEPQIIT |
| | | TDNTFVSGNCDVVIGIVNNTVYDPLQPELDSFKEELDKYFKNHTSPD |
| | | VDLGDISGINASVVNIQKEIDRLNEVAKNLNESLIDLQELGKYEQ |
| 61 | VFLIP_BA4.Beta | QCVNLITRTQSYTNSFTRGVYYPDKVFRSSVLHSTQDLFLPFFSNVTW |
| | | FHAISGTNGTKRFDNPVLPFNDGVYFASTEKSNIIRGWIFGTTLDSKT |
| | | QSLLIVNNATNVVIKVCEFQFCNDPFLDVYYHKNNKSWMESEFRVY |
| | | SSANNCTFEYVSQPFLMDLEGKQGNFKNLREFVFKNIDGYFKIYSKH |
| | | TPINLGREPEDLPQGFSALEPLVDLPIGINITRFQTLLALHRSYLTPGDS |
| | | SSGWTAGAAAYYVGYLQPRTFLLKYNENGTITDAVDCALDPLSETK |
| | | CTLKSFTVEKGIYQTSNFRVQPTESIVRFPNITNLCPFDEVFNATRFAS |
| | | VYAWNRKRISNCVADYSVLYNFAPFFAFKCYGVSPTKLNDLCFTNV |
| | | YADSFVIRGNEVSQIAPGQTGNIADYNYKLPDDFTGCVIAWNSNKLD |
| | | SKVGGNYNYRYRLFRKSNLKPFERDISTEIYQAGNKPCNGVAGVNC |
| | | YFPLQSYGFRPTYGVGHQPYRVVVLSFELLHAPATVCGPKKSTNL VK |
| | | NKCVNFNFNGLTGTGVLTESNKKFLPFQQFGRDIADTTDAVRDPQTL |
| | | EILDITPCSFGGVSVITPGTNTSNQVAVLYQGVNCTEVPVAIHADQLT |
| | | PTWRVYSTGSNVFQTRAGCLIGAEYVNNSYECDIPIGAGICASYQGG |
| | | SGGSSIIAYTMSLGAENSVACSNNSIAIPTNFTISVTTEILPVSMTKTSV |
| | | DCTMYICGDSTECSNLLLQYGSFCTQLKRALTGIAVEQDKNTQEVFA |
| | | QVKQIYKTPPIKYFGGFNFSQILPDPSKPSKRSPIEDLLFNKVTLADAG |
| | | FIKQYGDCLGDIAARDLICAQNFKGLTVLPPLLTDEMIAQYTSALLAG |
| | | TICSGWTFGAGPALQIPFPMQMAYRFNGIGVTQNVLYENQKLIANQF |
| | | NSAIGKIQDSLSSTPSALGKLQDVVNHNAQALNTLVKQLSSKFGAISS |
| | | VLNDILSRLDKPEAEVQIDRLITGRLQSLQTYVTQQLIRAAEIRASANL |
| | | AATKMSECVLGQSKRVDFCGKGYHLMSFPQSAPHGVVFLHVTYVP |
| | | AQEKNFTTAPAICHDGKAHFPREGVFVSNGTHWFVTQRNFYEPQIIT |
| | | TDNTFVSGNCDVVIGIVNNTVYDPLQPELDSFKEELDKYFKNHTSPD |
| | | VDLGDISGINASVVNIQKEIDRLNEVAKNLNESLIDLQELGKYEQ |
| 62 | VFLIP_BQ.1.1 | QCVNLITRTQSYTNSFTRGVYYPDKVFRSSVLHSTQDLFLPFFSNVTW |
| | | FHAISGTNGTKRFDNPVLPFNDGVYFASTEKSNIIRGWIFGTTLDSKT |
| | | QSLLIVNNATNVVIKVCEFQFCNDPFLDVYYHKNNKSWMESEFRVY |
| | | SSANNCTFEYVSQPFLMDLEGKQGNFKNLREFVFKNIDGYFKIYSKH |
| | | TPINLGREPEDLPQGFSALEPLVDLPIGINITRFQTLLALHRSYLTPGDS |
| | | SSGWTAGAAAYYVGYLQPRTFLLKYNENGTITDAVDCALDPLSETK |
| | | CTLKSFTVEKGIYQTSNFRVQPTESIVRFPNITNLCPFDEVFNATTFAS |
| | | VYAWNRKRISNCVADYSVLYNFAPFFAFKCYGVSPTKLNDLCFTNV |
| | | YADSFVIRGNEVSQIAPGQTGNIADYNYKLPDDFTGCVIAWNSNKLD |
| | | STVGGNYNYRYRLFRKSKLKPFERDISTEIYQAGNKPCNGVAGVNCY |
| | | FPLQSYGFRPTYGVGHQPYRVVVLSFELLHAPATVCGPKKSTNLVKN |
| | | KCVNFNFNGLTGTGVLTESNKKFLPFQQFGRDIADTTDAVRDPQTLE |
| | | ILDITPCSFGGVSVITPGTNTSNQVAVLYQGVNCTEVPVAIHADQLTP |
| | | TWRVYSTGSNVFQTRAGCLIGAEYVNNSYECDIPIGAGICASYQGGS |
| | | GGSSIIAYTMSLGAENSVACSNNSIAIPTNFTISVTTEILPVSMTKTSVD |
| | | CTMYICGDSTECSNLLLQYGSFCTQLKRALTGIAVEQDKNTQEVFAQ |
| | | VKQIYKTPPIKYFGGFNFSQILPDPSKPSKRSPIEDLLFNKVTLADAGFI |
| | | KQYGDCLGDIAARDLICAQNFKGLTVLPPLLTDEMIAQYTSALLAGTI |
| | | CSGWTFGAGPALQIPFPMQMAYRFNGIGVTQNVLYENQKLIANQFN |
| | | SAIGKIQDSLSSTPSALGKLQDVVNHNAQALNTLVKQLSSKFGAISSV |
| | | LNDILSRLDKPEAEVQIDRLITGRLQSLQTYVTQQLIRAAEIRASANLA |
| | | ATKMSECVLGQSKRVDFCGKGYHLMSFPQSAPHGVVFLHVTYVPA |
| | | QEKNFTTAPAICHDGKAHFPREGVFVSNGTHWFVTQRNFYEPQIITT |
| | | DNTFVSGNCDVVIGIVNNTVYDPLQPELDSFKEELDKYFKNHTSPDV |
| | | DLGDISGINASVVNIQKEIDRLNEVAKNLNESLIDLQELGKYEQ |

### Immunogenic Conjugates

One aspect of the present disclosure provides an immunogenic conjugate comprising, consisting of, or consisting essentially of a self-assembling nanoparticle comprising the amino acid sequence of a recombinant Ketol-acid reductoisomerase (KARI; EC 1.1.1.86), and a tether sequence; a protein of interest comprising a capture sequence. In some embodiments, the capture sequence and the tether sequence form a covalent bond that operably links the self-assembling nanoparticle to the protein of interest.

The novel nanoparticle platform of the present disclosure (e.g., KARI nanoparticle) can act as a self-assembling multivalent protein platform for the delivery of a plurality of antigens (e.g., different antigens, a repetitive array of antigens and/or numerous antigens) on the same nanoparticle that can be the same or different. For example, a KARI nanoparticle as described herein can comprise a plurality of antigens, which are the same (i.e., the KARI nanoparticle displays a single type of antigen). In other embodiments, the KARI nanoparticle can comprise a plurality of antigens, which are different (e.g., the KARI nanoparticle may display several types of antigens).

The plurality of antigens may each be operably linked to a monomer of the dodecameric KARI particle disclosed herein. For example, one antigen per monomer for a total of 12 antigens on a single dodecameric KARI nanoparticle that can be the same or different. When the plurality of antigens are the same, the KARI nanoparticle can display a single type of antigens. When the plurality of antigens are different, the KARI nanoparticle can display several types of antigens. If the antigens are different, the dodecameric KARI particle can comprise up to twelve different antigens. For example, at least about 2, at least about 3, at least about 4, at least about 5, at least about 6, at least about 7, at least about 8, at least about 9, at least about 10, at least about 11, and/or at least about 12 different antigens can be operably linked to a single dodecameric KARI particle.

In another embodiment, each monomer of the dodecameric KARI nanoparticle can operably be linked to a plurality of antigens, which can be the same or different. For example, each monomer of the dodecameric KARI particle can be operably linked to one or more antigens, where the antigens are either the same or different. For example, each monomer can be linked to 2, 3, 4, 5, 6, 7, 8, 9 or more antigens that can be the same or different. In that embodiment, the KARI nanoparticle can display at least about 24, at least about 36, at least about 48, at least about 60, at least about 72, at least about 84, at least about 96, or at least about 108 or more antigens, where the antigens are the same or different.

In some embodiments, the KARI nanoparticle comprises 2 antigens per KARI monomer and/or the two antigens are the same or different. In some embodiments, the KARI nanoparticle comprises 3 antigens per KARI monomer and/or the three antigens are the same or different. In some embodiments, the KARI nanoparticle comprises 4 antigens per KARI monomer and/or the four antigens are the same or different. In some embodiments, the KARI nanoparticle comprises 5 antigens per KARI monomer and/or the five antigens are the same or different. In some embodiments, the KARI nanoparticle comprises 6 antigens per KARI monomer and/or the six antigens are the same or different. When the antigens are different, they can be from the same species or different species; or they can target the same epitope or different epitopes on the same receptor and/or cell. In other embodiments, the different antigens can target different receptors on different cell types.

In a particular embodiment, the KARI nanoparticles disclosed herein can be used as a multivalent system to deliver various proteins of interests including, but not limited to T-cell epitopes. The KARI nanoparticles disclosed herein can be used to display different molecules of interest (e.g., antigens, antibodies, drugs, adjuvants) via a coupling system (e.g., SpyTag/SpyCatcher system, Sortase A system) to generate a multivalent system capable of delivering T-cell epitopes or any short peptides (e.g., antigens).

### Immunogen Polypeptide

In some embodiments, the protein of interest is an immunogen polypeptide. Any immunogen polypeptide can be used in the self-assembling KARI nanoparticle of the present disclosure. These include any proteins or polypeptides from pathogens against which an elicited immune response may be desired. Thus, the immunogenic compositions of the disclosure can utilize immunogen polypeptides (e.g., protein of interest) that are derived from any tumors, viruses, bacteria, or other pathogenic organisms. Suitable immunogen polypeptides for the present disclosure can also be derived from non-pathogenic species, including human proteins, against which an elicited immune response may have a therapeutic effect, alleviate disease symptoms, or improve general health. In some embodiments, the protein is a tumor antigen.

In general, the immunogen polypeptide can be any structural or functional polypeptide or peptide that contains at least about 10 amino acid residues. In some embodiments, the immunogen polypeptides contain between about 10 to about 10,000 amino acid residues in length. In some embodiments, the immunogen polypeptides contain between about 25 to about 2,000 amino acid residues in length. In some embodiments, the immunogen polypeptides contain about 50 to about 500 amino acid residues in length. Thus, the immunogen polypeptides or proteins suitable for the invention can have a molecular weight of from about 1 kDa to about 1,000 kDa, and preferably from about 2.5 kDa to about 250 kDa. In some more preferred embodiments, the employed immunogen polypeptide has a molecular weight of about 5 kDa to about 25 kDa or 50 kDa.

In some embodiments, the immunogen polypeptide or protein used in the self-assembly nanoparticle and compositions thereof of the present disclosure can be derived from a viral surface or core protein (target polypeptide).

The immunogen polypeptide is selected from an antigen, a tumor antigen, a viral antigen, a fungal antigen, a bacterial antigen, an antigen for the development of a vaccine, an antibody, an antibody fragment, a single chain antibody, scFv, scFab, single domain antibody, a protein scaffolds, an enzyme, a hormone, or an interleukin.

### SARS-CoV-2 and Variants

A central goal for SARS-CoV-2 vaccines is to reduce incidence of symptomatic disease through generation of enduring protective immunity. However, the recent emergence of SARS-CoV-2 variants of concern (VOC) poses a risk to first-generation vaccine efficacy and durability of both infection- and vaccine-induced humoral immunity. Lineage B.1.351 (informally known as the South African variant) is particularly concerning due to substitutions that confer increased transmissibility and reduced sensitivity to neutralization by heterotypic convalescent and vaccine-induced sera. Development of structurally designed vaccine candidates with improved immunogenicity and breadth of coverage is critical for controlling emergent VOC. To address these issues associated with current spike constructs and emergence of VOC, numerous generations of spike antigens for used in vaccine have been developed.

The first-generation designs to improve SARS-CoV-2 introduced two proline residues to stabilize the prefusion conformation, termed SARS S 2P. Vaccines currently deployed in the United States use a derivative of the prototypical, first-generation “S-2P” spike design (Pallesen et al. 2017), which contains two proline substitutions at positions 986 and 987 (Polack et al. 2020; Bos et al. 2020; Corbett et al. 2020; Wrapp et al. 2020). These vaccines have shown high efficacy in the short term, but the rapid timeframe for development has afforded few opportunities for antigen optimization. Recent work by Hsieh et al. illustrated that the S-2P spike exhibits relatively low yield and unfavorable purity (Hsieh et al. 2020). The poor yield may impact cost and manufacturability of vaccine candidates, and limit expression levels in vaccinated individuals, which in turn could necessitate higher doses and potentially increased reactogenicity. Moreover, several studies reported that S-2P protein preparations exhibit sensitivity to cold-temperature storage (Edwards et al. 2020; Xiong et al. 2020). Edwards et al. used negative stain electron microscopy (NSEM) to demonstrate a 95% loss of well-formed S-2P spike trimers after 5-7 days of storage at 4° C. Exposure to 4° C. temperatures also resulted in lower thermostability and altered binding to monoclonal antibody (mAb) CR3022, suggesting perturbed structure and antigenicity.

The second-generation SARS-CoV-2 S vaccine antigen termed HexaPro, further stabilizes the antigen by introduction of four more proline residues. The second-generation spike construct, termed “HexaPro”, contains four additional prolines at positions 817, 892, 899 and 942. HexaPro expresses to levels nearly 10-fold higher than those for wild-type spike or S-2P, has a 5° C. higher melting temperature (Tm) (Hsieh et al. 2020), and displays improved stability relative to S-2P under low-temperature storage and multiple freeze-thaw cycles (Edwards et al. 2020). Importantly, binding assays and cryoEM indicated that HexaPro better retains the native prefusion quaternary structure compared to S-2P, despite still exhibiting minor reductions in thermostability and mAb binding following incubation at 4° C.

A third generation SARS-CoV-2 S vaccine antigen where the spike protein contains different proline substitutions, cleavage site linkers, and interprotomer disulfide bonds. See e.g., WO2022087255A2, which is incorporated herein in its entirety. The third generation of SARS-CoV-2 vaccine improved through iterative cycles of rational structure-based design that significantly increased both the transient expression yield of the antigen as well as its stability in different physical conditions. As such, the third generation comprises USEQ DS' stabilized immunogens contain one or more of three improvements over the current state-of-the-art HexaPro: (1) have their SI/S2 subunits genetically linked by replacement of their furin cleavage site loops by short flexible or rigid linkers, (2) their interprotomeric movements stabilized by an additional introduced disulfide bond, and (3) deletion of one of the six prolines in HexaPro (yielding PentaPro, but also 1, 2, 3 or 4 changes to or from proline) for greater trimeric prefusion stability. These USEQ DS immunogens maintain the structural characteristics corresponding to an uncleaved prefusion-stabilized S glycoprotein with a substantial improvement in the stability of the trimer against inactivation by heat, by freeze/thaw cycles and lyophilization/resuspension of the protein.

The third generation SARS-CoV-2 S vaccine antigen termed “VFLIP” (five (V) prolines, Flexibly-Linked, Inter-Protomer disulfide) spikes. In the “VFLIP”, the spikes remain trimeric without exogenous trimerization motifs, and which have enhanced thermostability relative to earlier spike constructs. Surface plasmon resonance (SPR) and cryo-EM analysis confirm the native-like antigenicity of VFLIP and its improved utility for structural biology applications. Moreover, mice immunized with the VFLIP spike elicited significantly more potent neutralizing antibody responses against live SARS-CoV-2 D614G and B.1.351 compared to those immunized with S-2P. The VFLIP is a thermostable, covalently linked, native-like spike trimer that represents a next-generation research reagent, diagnostic tool, immunogen, and vaccine.

In some embodiments, the protein of interest is a viral antigen selected from a coronavirus. In some embodiments, the protein of interest is a viral antigen derived from SARS-CoV-1, MERS-CoV, 229E (alpha), NL63 (alpha), OC43 (beta), HKU1 (beta), or SARS-CoV-2. In those embodiments, the protein of interest is a viral antigen derived from a SARS-CoV-2. In those embodiments, the viral antigen is derived from a SARS-CoV-2 variant selected from B.1.1.7, B.1.1.7 with E484K, B.1.135, B.1.351, P.1, B.1.427, D614G, B.1.1351, B.1.429, Lambda (C.37), or Mu (B.1.621).

In some embodiments, the protein of interest is a coronavirus spike protein. In some embodiments, the protein of interest is a mutant coronavirus spike protein. In some embodiments of the self-assembling nanoparticle described herein, the protein of interest is a first-generation “S-2P” spike comprising two proline substitutions at positions 986 and 987 of the spike protein. In some embodiments of the self-assembling nanoparticle described herein, the protein of interest is a second-generation “HexaPro”, comprising four additional prolines at positions 817, 892, 899 and 942 of the Spike protein. In some embodiments of the self-assembling nanoparticle described herein, the protein of interest is a third generation VFLIP” spike protein.

In some embodiments of the self-assembling nanoparticle described herein, the protein of interest is a mutant coronavirus spike protein comprising 1, 2, 3, 4, or 5 proline mutations selected from F817P, A892P, A899P, A942P, P986K, K986P, V987P, and P987V. In some embodiments, the protein of interest is a mutant coronavirus spike protein comprising at least one additional disulfide bond selected from F43C-G566C, G413C-P987C, Y707C-T883C, G1035C-V1040C, A701C-Q787C, G667C-L864C, V382C-R983C, and I712C-I816C.

In some embodiments, the mutant coronavirus spike protein comprises, consists of or consists essentially of the amino acid sequence of SEQ ID NO: 11 or an amino acid sequence having at least about 70%, at least about 75%, at least about 80%, at least about 85%, at least about 90%, at least about 95%, at least about 97%, or at least about 99% identity to the amino acid sequence of SEQ ID NO:11. In some embodiments, the mutant coronavirus spike protein comprises the amino acid sequence of a spike protein disclosed in, for example, WO 2022/087255A2, which is incorporated by reference in its entirety.

The novel nanoparticle platform of the present disclosure allows for the possibility to deliver different antigens (i.e., different SARS-CoV-2 variants) on the same nanoparticle by mixing all the antigens in equimolar concentration. For example, the dodecameric nanoparticle of the present disclosure allows, for example, the S trimers derived from one or more coronaviruses or SARS-CoV-2 variants to be displayed on the same dodecameric nanoparticle platforms, with a size ranging from 12.2 to 50.0 nm. Alternatively, the different antigens may each be operably linked to a monomer of dodecameric KARI particle. For example, each monomer may be linked to 2, 3, 4, 5, or more S trimers derived from the same or different coronaviruses or SARS-CoV-2 variants. As such, an immunogenic conjugate disclosed herein may comprise a KARI nanoparticle that may display at least about 12, at least about 24, at least about 36, at least about 48, at least about 60, at least about 72, at least about 84, at least about 96, or at least about 108 S trimers derived from one or more coronaviruses or SARS-CoV-2 variants where the S trimers may be the same or different.

### Additional Viral Immunogens

In some embodiments, the immunogen polypeptide or protein used in the vaccine compositions of the invention can be derived from a viral surface or core protein (target polypeptide). There are many known viral proteins that are important for viral infection of host cells. Examples include, but are not limited to, glycoproteins (or surface antigens, e.g., GP120 and GP41) and capsid proteins (or structural proteins, e.g., P24 protein) of HIV; surface antigens or core proteins of hepatitis A, B, C, D or E virus (e.g., small hepatitis B virus surface antigen (S-HBsAg) and the core proteins of hepatitis C virus, NS3, NS4 and NS5 antigens); glycoproteins gp350/220 of Epstein-Barr virus (EBV), glycoprotein (G-protein) or the fusion protein (F-protein) of respiratory syncytial virus (RSV); surface and core proteins of herpes simplex virus HSV-1 and HSV-2 (e.g., glycoprotein D from HSV-2), surface proteins (e.g., gB, gC, gD, gH and gL) of poliovirus, envelope glycoproteins hemagglutinin (H) and fusion protein (F) of measles virus (MV), glycoprotein G of lymphocytic choriomeningitis virus (LCMV), fiber and penton base proteins of adenoviruses, S spikes of coronaviruses, envelope (E) proteins of flaviviruses such as Dengue virus, yellow fever virus, and Zika virus, and non-enveloped capsid proteins of picornaviruses.

In some embodiments, viral immunogens suitable for the present disclosure can be derived from viruses utilizing the class-I fusion mechanism for infection. Class-I viral fusion proteins are trimers that will undergo dramatic conformational changes during cell entry. Specific regions in the viral protein can completely refold to facilitate membrane fusion. As exemplified herein, examples of immunogens of viruses utilizing class-I fusion mechanism include structural proteins or polypeptides obtained from HIV-1, from viruses that cause hemorrhagic fevers such as Filoviruses (e.g., Ebola virus and Marburg viruses) and Arenaviruses (e.g., Lassa virus), from respiratory syncytial virus (RSV), and from coronaviruses such as MERS-CoV and SARS-CoV.

Suitable immunogens can be any proteins and polypeptides that are derived from HIV-1 UFO trimmer, Ebola GP ectodomain, LASV glycoprotein complex (GPC), RSV glycoprotein F, and MERS-CoV spike protein S. Any of these immunogens, or immunogens derived from structural proteins of other viruses utilizing the class-I fusion mechanism, can all be employed in the KARI nanoparticle design of the present disclosure.

In some embodiments, viral immunogens suitable for use with the KARI nanoparticle can be derived from viruses utilizing the class-II fusion mechanism for infection. Class-II viral fusion proteins exist in the form of heterodimer (e.g., hepatitis C virus) or homodimer (e.g., Dengue and Zika viruses), which will refold to form a trimeric spike prior to membrane fusion. As exemplified herein, suitable immunogens can be any proteins and polypeptides that are derived from HCV envelope glycoproteins (e.g., E2), Zika virus E protein (e.g., the Dill domain) or any structural proteins of other viruses utilizing the class-II fusion mechanism.

### Non-Viral Immunogens

In some embodiments, the immunogen polypeptide or protein used in the KARI nanoparticle or composition thereof of the present disclosure can be derived from a non-viral target. These include immunogens that can be obtained from any non-viral pathogens (e.g., bacterial pathogens) as well as parasitic organisms inside mammalian hosts such as human. In some embodiments, bacterial proteins that are important for bacterial infections are suitable for obtaining immunogen polypeptides in the vaccine design of the invention. Suitable immunogens can be any proteins and polypeptides that are derived from structural proteins of the bacteria, e.g., Ag85 complex and Mtb72 as exemplified herein with Mycobacterium tuberculosis (TB). In some embodiments, parasitic proteins that are important for parasite transmission, reproduction in the hosts, and life cycle are suitable for obtaining immunogen polypeptides in the vaccine design of the invention. Suitable immunogens can be any proteins and polypeptides that are derived from structural proteins of the parasites, e.g., Pfs25, circumsporozoite protein (CSP), and reticulocyte binding protein homolog 5 (PfRH5) of Plasmodium falciparum (Malaria).

### Tumor Antigens

In some embodiments, the employed immunogen polypeptide can be an endogenous protein from a mammalian host (e.g., human), against which an elicited immune response is desired. These include, e.g., PCSK9 for regulating cholesterol level as exemplified herein, and ghrelin for controlling appetite. Various other mammalian proteins can also be used to obtain suitable immunogen polypeptides for constructing the immunogenic conjugate described herein. In some embodiments, the immunogen polypeptide can be other proteins implicated in human diseases. These include proteins that are involved in the development of cancers. Examples of cancer related immunogens also include non-mutated self-antigens, e.g., MAGE-A3, Melan-A/Martl, gp100, Her2/Neu, and NY-ESO-l.

In some embodiments, the employed immunogen polypeptide is a tumor antigen selected from alpha fetoprotein (AFP)/HLA-A2, AXL, B7-H3, BCMA, CA-IX, CD2, CD3, CD4, CDS, CD7, CD8, CD19, CD20, CD22, CD30, CD33, CD38, CD44v6, CD70, CD79a, CD79b, CD80, CD86, CDI 17, CD123, CD133, CD147, CDI 71, CD276, CEA, claudin 18.2, c-Met, DLL3, DRS, EGFR, EGFRvlll, EpCAM, EphA2, FAP, folate receptor alpha (FRa)/folate binding protein (FBP), GD-2, Glycolipid F77, glypican-3 (GPC3), HER2, HLA-A2, ICAMI, IL3Ra, IL13Ra2, LAGE-I, Lewis Y, LMPI (EBV), MAGE-A1, MAGE-A3, MAGE-A4, Melan A, mesothelin, MP7 (glycosylated CEA), MMP, MUCI, Nectin4/FAP, NKG2D-Ligands (MIC-A, MIC-B, and the ULBPs I to 6), NY-ESO-1, P16, PD-LI, PSCA, PSMA, RORI, ROR2, TIM-3, TM4SF1, TnMuc1, or VEGFR2.

In some additional embodiments, the immunogen polypeptides or proteins for use in immunogenic conjugate of the present disclosure include proteins implicated in other chronic human diseases or disorders. Examples of such human target include, e.g., Ang-II for hypertension, TNF-α for inflammations, IL-9 for pathogen-induced eosinophilia, IL-5 for asthma, N-methyl-D-aspartate receptor-1 for stroke and human chorionic gonadotropin (hCG) for decreasing hormone levels.

### KARI Immunogenic Conjugates

In some embodiments, the immunogenic conjugate further comprising a T-cell epitope. In that embodiment, the T-cell epitope is operably linked to the C-terminus or the N-terminus of the recombinant KARI. In some embodiments, the recombinant KARI is a dodecameric KARI.

In some embodiments of the immunogenic conjugate described herein, the recombinant KARI is an archeal, bacterial, or proteobacteria KARI. In some embodiments of the immunogenic conjugate described herein, the recombinant KARI is selected from Methanothermococcus thermolithotrophicus (MtKARI), Helicobacter pylori, Pseudomonas Aeruginosa (PaKARI), Saccharolobus solfataricus (SacsKARI), Sulfolobus solfataricus (Sso-KARI), or A. vinelandii, Sulfolobus sp. E5-1-F, Sulfolobus islandicus, Saccharolobus shibatae, Saccharolobus caldissimus, Saccharolobus shibatae, Stygiolobus sp. KN-1, Sulfolobus sp., Sulfodicoccus acidiphilus, Acidianus brierleyi, or Acidianus manzaensis; or (c) is a dodecameric KARI selected from M. thermolithotrophicus (MtKARI), Helicobacter pylori, Pseudomonas. Aeruginosa (PaKARI), Saccharolobus solfataricus (SacsKARI), Sulfolobus solfataricus (Sso-KARI), or A. vinelandii.

In some embodiments of the immunogenic conjugate described herein, the recombinant KARI the recombinant KARI oligomerizes into a dodecameric (12-mer) nanoparticle. In some embodiments, the recombinant KARI oligomerizes into a dodecameric KARI nanoparticle and displays the protein of interest on the surface of the nanoparticle. In some embodiments, the dodecameric KARI nanoparticle displays at least about twelve molecules of the protein of interest on the surface of the dodecameric KARI nanoparticle.

In some embodiments of the immunogenic conjugate described herein, the recombinant KARI comprises, consists of or consists essentially of the amino acid sequence of SEQ ID NO: 1 or 2, or an amino acid sequence having at least about 37%, at least about 40%, at least about 50%, at least about 55%, at least about 65%, at least about 70%, at least about 75%, at least about 80%, at least about 85%, at least about 90%, at least about 95%, or at least about 99% identity to the amino acid sequence of SEQ ID NO: 1 or 2.

In some embodiments, the recombinant KARI comprises a deletion in the amino acid sequence of SEQ ID NO: 1. The deletion enhances the expression and purification of the self-assembly nanoparticle when compared to the expression and purification of a wild-type KARI nanoparticle. In some embodiments, the recombinant KARI comprises a deletion of about 2, about 3, about 5, about 10, about 12, about 15, or about 20 amino acids in the N-terminus of SEQ ID NO: 1. In some embodiments, the recombinant KARI comprises the amino acid sequence of SEQ ID NO: 2.

In some embodiments of the immunogenic conjugate described herein, the tether sequence is selected from SpyTag, SpyTag001, SpyTag002, SpyTag003, SpyCatcher, SpyCatcher001, SpyCatcher002, SpyCatcher003, SnoopTag, SnoopCatcher, a sortase recognition domain, a sortase bridging domain, a butelase recognition motif, a C-peptide, a split intein, or a peptiligase substrate.

In some embodiments, the tether sequence comprises, consists essentially of, or consists of the amino acid sequence of SEQ ID NO: 4, 8, 13, 15, or 28-39 or an amino acid sequence having at least about 70%, at least about 75%, at least about 80%, at least about 85%, at least about 90%, at least about 95%, at least about 97%, or at least about 99% identity to the amino acid sequence of SEQ ID NO: 4, 8, 13, 15, or 28-39 (across the full length of the amino acid sequence).

In some embodiments, the capture sequence is selected from SpyTag, SpyTag001, SpyTag002, SpyTag003, SpyCatcher, SpyCatcher001, SpyCatcher002, SpyCatcher003, SnoopTag, SnoopCatcher, a sortase recognition domain, a sortase bridging domain, a butelase recognition motif, a C-peptide, a split intein, or a peptiligase substrate. In some embodiments, the tether sequence is selected from SpyTag, SpyTag001, SpyTag002, SpyTag003, SpyCatcher, SpyCatcher001, SpyCatcher002, SpyCatcher003, SnoopTag, SnoopCatcher, a sortase recognition domain, a sortase bridging domain, a butelase recognition motif, a C-peptide, a split intein, or a peptiligase substrate.

In some embodiments, the capture sequence comprises, consists essentially of, or consists of the amino acid sequence of SEQ ID NO: 4, 8, 13, 15, or 28-39 or an amino acid sequence having at least about 70%, at least about 75%, at least about 80%, at least about 85%, at least about 90%, at least about 95%, at least about 97%, or at least about 99% identity to the amino acid sequence of SEQ ID NO: 4, 8, 13, 15, or 28-39.

In some embodiments of the immunogenic conjugate disclosed herein, the self-assembling nanoparticle further comprises a linker. In some embodiments, the linker operably links the recombinant KARI to the tether or capture sequence. In some embodiments, the linker operably links the protein of interest (e.g., an antigen) to the tether or capture sequence. In some embodiments, the linker is a glycine-serine linker or the linker comprises, consists of, or consists essentially of the amino acid sequence of SEQ ID NO: 5, 9, 16-20, 44, or 52-58 or an amino acid sequence having at least about 70%, at least about 75%, at least about 80%, at least about 85%, at least about 90%, at least about 95%, at least about 97%, or at least about 99% identity to the amino acid sequence of SEQ ID NO: 5, 9, 16-20, 44, 52-58 (across the full length of the amino acid sequence).

In some embodiments, the linker is selected from at least one of: GGS, GP, GPGP (SEQ ID NO:54), GGSGGS (SEQ ID NO:55), GGGS (SEQ ID NO:56) or GGGSGGGS (SEQ ID NO:57). In some embodiments, the linker sequence is repeated 1, 2, 3, 4, or 5 times. In still other embodiments, the linker comprises, consists, or consists essentially of: GGGSGGGSGGGSGGGS (SEQ ID NO: 58). In some embodiments, the linker is preferably GGGSGGGSGGGSGGGS (GGGS)4 (SEQ ID NO: 58) or preferably comprises the amino acid sequence of SEQ ID NO: 58.

In some embodiments of the immunogenic conjugate disclosed herein, the self-assembling nanoparticle further comprises a linker. In some embodiments, the linker is a glycine-serine linker or the linker comprises an amino acid sequence of SEQ ID NO: 5 or 9 or an amino acid sequence having at least about 70%, at least about 75%, at least about 80%, at least about 85%, at least about 90%, at least about 95%, at least about 97%, or at least about 99% identity to the amino acid sequence of SEQ ID NO: 5 or 9 (across the full length of the amino acid sequence).

In some embodiments of the immunogenic conjugate disclosed herein, the immunogenic conjugate further comprises a purification tag. In one embodiment, the purification tag is operably linked to a C-terminus or an N-terminus of the recombinant KARI. In some embodiments, the purification tag is selected from hexahistidine tag (SEQ ID NO: 65) (his-tag), Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin). In some embodiments, the purification tag comprises the amino acid sequence of SEQ ID NO: 6 or an amino acid sequence having at least about 70%, at least about 75%, at least about 80%, at least about 85%, at least about 90%, at least about 95%, at least about 97%, or at least about 99% identity to the amino acid sequence of SEQ ID NO: 6 (across the full length of the amino acid sequence).

In some embodiments of the immunogenic conjugate disclosed herein, the immunogenic polypeptide comprises additional structural components, such as the locking domain, trimerization motifs, linkers, T-cell epitopes. These structural components as described herein. In some embodiments of the immunogenic conjugate disclosed herein, the immunogen polypeptide is operably linked to a purification tag, and/or a trimerization motif. In some embodiments, the trimerization motif is a T4 fibritin foldon, an HIV-I -derived molecular clamp, or a viral capsid protein SHP.

In some embodiments, the trimerization motif comprises the amino acid sequence of SEQ ID NO: 12, 21, or 22 or an amino acid sequence having at least about 70%, at least about 75%, at least about 80%, at least about 85%, at least about 90%, at least about 95%, at least about 97%, or at least about 99% identity to the amino acid sequence of SEQ ID NO: 12, 21, or 22.

In some embodiments of the immunogenic conjugate disclosed herein, the immunogenic polypeptide comprises further comprises a T-cell epitope operably linked to the C-terminus or the N-terminus of the recombinant KARI.

In some embodiments, the T-cell epitope is selected from a universal DR epitope (PADRE) T-helper epitope, a CpG-oligodeoxynucleotides (CpG-ODNs), a multi-epitope long peptide, a SARS-CoV-2 nucleo capsid protein N-terminal domain, a SARS-CoV-2 nucleoprotein T-cell epitope. In some embodiments, the T-cell epitope comprises the amino acid sequence of SEQ ID NO: 23-27 or an amino acid sequence having at least about 70%, at least about 75%, at least about 80%, at least about 85%, at least about 90%, at least about 95%, at least about 97%, or at least about 99% identity to the amino acid sequence of SEQ ID NO: 23-27 (across the full length of the amino acid sequence). In some embodiments, the T-cell epitope comprises a SARS-CoV-2 nucleoprotein epitope comprising the amino acid sequence of SEQ ID NO: 26 or 27. In some embodiments, the T-cell epitope is a as a CD4⁺ T-helper epitope or a CD8⁺ T-cell epitope.

### Nucleic Acid and Vectors

One aspect of the present disclosure provides polynucleotides encoding a disclosed recombinant KARI protein and/or the self-assembling nanoparticle described herein. These polynucleotides include DNA, cDNA and RNA sequences which encode a disclosed recombinant KARI protein and/or the self-assembling nanoparticle. In some embodiments, the present disclosure provides a nucleic acid encoding the self-assembly nanoparticle described herein. In some embodiments, the present disclosure provides a nucleic acid encoding the immunogenic conjugate described herein.

One of skill in the art can readily use the genetic code to construct a variety of functionally equivalent nucleic acids, such as nucleic acids which differ in sequence but which encode the same recombinant KARI protein and/or the self-assembling nanoparticle d sequence, or encode a conjugate or fusion protein thereof including the nucleic acid sequence.

In several embodiments, the nucleic acid molecule encodes a precursor of a disclosed recombinant KARI protein and/or the self-assembling nanoparticle described herein, that, when expressed in an appropriate cell, is expressed and processed into a recombinant KARI protein and/or the self-assembling nanoparticle described herein fused to a protein of interest, such e.g., a SARS-CoV2-spike protein or recombinant coronavirus spike protein as described herein. For example, the nucleic acid molecule can encode a recombinant KARI protein that includes a N-terminal signal peptide for entry into the cellular secretory system that is proteolytically cleaved in the during processing of the recombinant protein in the cell. In another example, the nucleic acid molecule can encode a recombinant KARI protein that includes a N-terminal or a C-terminal a T-cell epitope or a tag protein as described herein. In another example, the nucleic acid molecule can encode a protein of interest that includes a N-terminal or a C-terminal purification tag and/or trimerization motif as described herein

### Generating the Nucleic Acid

Exemplary nucleic acids can be prepared by cloning techniques. Examples of appropriate cloning and sequencing techniques, and instructions sufficient to direct persons of skill through many cloning exercises are known (see e.g., Sambrook et al. (Molecular Cloning: A Laboratory Manual, 4^{th} ed, Cold Spring Harbor, N.Y., 2012) and Ausubel et al. (In Current Protocols in Molecular Biology, John Wiley & Sons, New York, through supplement 104, 2013). Product information from manufacturers of biological reagents and experimental equipment also provide useful information. Such manufacturers include the SIGMA Chemical Company (Saint Louis, Mo.), R&D Systems (Minneapolis, Minn.), Pharmacia Amersham (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (Carlsbad, Calif.), and Applied Biosystems (Foster City, Calif.), as well as many other commercial sources known to one of skill.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

The polynucleotides encoding a recombinant KARI protein or a self-assembling nanoparticle described herein can include a recombinant DNA which is incorporated into a vector (such as an expression vector) into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA.

Polynucleotide sequences encoding a recombinant KARI protein or a self-assembling nanoparticle can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA and stop codons.

Modifications can be made to a nucleic acid encoding a recombinant KARI protein or a self-assembling nanoparticle described herein without diminishing its biological activity. Some modifications can be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids, such as purification tags described herein to aid in purification steps.

In addition to recombinant methods, the recombinant KARI protein or a self-assembling nanoparticle described herein can also be constructed in whole or in part using protein synthesis methods known in the art.

### Host Cells

DNA sequences encoding a recombinant KARI protein or a self-assembling nanoparticle can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Hosts include microbial, yeast, insect and mammalian organisms. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see e.g., Helgason and Miller (Eds.), 2012, Basic Cell Culture Protocols (Methods in Molecular Biology), 4^{th} Ed., Humana Press). Examples of commonly used mammalian host cell lines are VERO and HeLa cells, CHO cells, and WI38, BHK, and COS cell lines, although cell lines may be used, such as cells designed to provide higher expression, desirable glycosylation patterns, or other features. In some embodiments, the host cells include HEK293 cells or derivatives thereof, such as GnTI^{-/-} cells (ATCC® No. CRL-3022), or HEK-293F cells.

Transformation of a host cell with recombinant DNA can be carried out by conventional techniques. Where the host is prokaryotic, such as, but not limited to, E. coli, competent cells which are capable of DNA uptake can be prepared from cells harvested after exponential growth phase and subsequently treated by the CaCl2)method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or viral vectors can be used. Eukaryotic cells can also be co-transformed with polynucleotide sequences encoding a disclosed antigen, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Viral Expression Vectors, Springer press, Muzyczka ed., 2011). One of skill in the art can readily use an expression systems such as plasmids and vectors of use in producing proteins in cells including higher eukaryotic cells such as the COS, CHO, HeLa and myeloma cell lines.

### Vectors

One aspect of the present disclosure provides a vector or a combination of vectors comprising the self-assembling KARI nanoparticle described herein. In one aspect the present disclosure provides a vector comprising the self-assembling KARI nanoparticles described herein. In some embodiments, the vector comprises a nucleic acid sequence encoding the recombinant KARI, and a nucleic acid sequence encoding the tether sequence. In some embodiments, the vector comprises a nucleic acid sequence encoding the recombinant KARI, a nucleic acid sequence encoding the tether sequence, and a nucleic acid encoding the purification Tag. In some embodiments, the vector comprises a nucleic acid sequence encoding a protein of interest and a nucleic acid sequence encoding the capture sequence.

In some embodiments, the vector comprises a nucleic acid sequence encoding the recombinant KARI, a nucleic acid sequence encoding the tether sequence, and a nucleic acid encoding the T-cell epitope. In some embodiments, the vector comprises a nucleic acid sequence encoding the recombinant KARI, a nucleic acid sequence encoding the tether sequence, a nucleic acid encoding the T-cell epitope, and a nucleic acid encoding the purification Tag. In some embodiments, the vector comprises a nucleic acid sequence encoding a protein of interest and a nucleic acid sequence encoding the capture sequence. In some embodiment, a vector comprising a nucleic acid sequence encoding the recombinant KARI protein is expressed in a prokaryotic expression system (e.g., a bacterial cell). In some embodiments, the vector comprising a nucleic acid sequence encoding a protein of interest is expressed in an Eukaryotic expression system.

In one aspect, the present disclosure provides a combination of vectors comprising the self-assembling nanoparticle described herein. In some embodiments, the combination of vectors comprises one or more vectors. In some embodiments the combination of vectors comprises a first vector and a second vector. In one embodiment, the first vector comprises a nucleic acid sequence encoding the recombinant KARI protein, and a nucleic acid sequence encoding a tether sequence; and the second vector comprises a nucleic acid sequence encoding a protein of interest and a nucleic acid sequence encoding the capture sequence. In another embodiment, the first vector comprises a nucleic acid sequence encoding the recombinant KARI protein, a nucleic acid sequence encoding the tether sequence, and a nucleic acid encoding a purification Tag; and the second vector comprises a nucleic acid sequence encoding a protein of interest and a nucleic acid sequence encoding the capture sequence. In some embodiments, the first vector encodes a polypeptide comprising the amino acid sequence of SEQ ID NO: 3 or 7. In some embodiments, the second vector encodes a polypeptide having the amino acid sequence of SEQ ID NO: 10 or 14. In some embodiments, the first and second vectors are brought together (e.g., ligated together). In that embodiment, the tether sequence of the first vector and the capture sequence of the second form a covalent bond with one another either spontaneously or with the help of an enzyme to generate an immunogenic polypeptide comprising the self-assembling nanoparticle fused to the protein of interest.

In some embodiments of the combination of vectors comprising the self-assembling nanoparticle described herein, the combination of vectors comprises: a first vector comprising a nucleic acid sequence encoding the recombinant KARI, a nucleic acid sequence encoding the tether sequence, and a nucleic acid encoding the T-cell epitope; and a second vector comprising a nucleic acid sequence encoding a protein of interest and a nucleic acid sequence encoding the capture sequence. In some embodiments, the combination of vectors comprises a first vector comprising a nucleic acid sequence encoding the recombinant KARI, a nucleic acid sequence encoding the tether sequence, a nucleic acid encoding the T-cell epitope, and a nucleic acid encoding the purification Tag; and a second vector comprising a nucleic acid sequence encoding a protein of interest and a nucleic acid sequence encoding the capture sequence.

In some embodiments of the combination of vectors described herein, the first vector is expressed in a prokaryotic expression system (e.g., a bacterial cell) and the second vector is expressed in an eukaryotic expression system (e.g., a mammalian cell line).

In some embodiments of the combination of vectors described herein, the first vector encodes a polypeptide comprising the amino acid sequence of SEQ ID NO: 3 or 7. In Some embodiments of the combination of vectors described herein, the second vector encodes a polypeptide having the amino acid sequence of SEQ ID NO: 10 or 14. In some embodiments of the combination of vectors described herein, the first vector encodes a polypeptide having the amino acid sequence of SEQ ID NO: 3 or 7 and the second vector encodes a polypeptide having the amino acid sequence of SEQ ID NO: 10 or 14.

In some embodiments of the combination of vectors described herein, when the first and second vectors are brought together, the tether sequence and the capture sequence form a covalent bond with one another either spontaneously or with the help of an enzyme to generate an immunogenic polypeptide comprising the self-assembling nanoparticle fused to the protein of interest.

The vectors of the present disclosure generally comprise transcriptional or translational control sequences required for expressing the protein of interest and a recombinant KARI protein. Suitable transcription or translational control sequences include but are not limited to replication origin, promoter, enhancer, repressor binding regions, transcription initiation sites, ribosome binding sites, translation initiation sites, and termination sites for transcription and translation.

The origin of replication (generally referred to as an ori sequence) permits replication of the vector in a suitable host cell. The choice of ori will depend on the type of host cells and/or genetic packages that are employed. Where the host cells are prokaryotes, the expression vector typically comprises two ori sequences, one directing autonomous replication of the vector within the prokaryotic cells, and the other ori supports packaging of the phage particles. Preferred prokaryotic ori can direct vector replication in bacterial cells. Non-limiting examples of this class of ori include pMB1, pUC, as well as other E. coli origins. Preferred ori supporting packaging of the phage particles includes but is not limited to fl ori, Pfi phage replication ori.

In the eukaryotic system, higher eukaryotes contain multiple origins of DNA replication, but the ori sequences are not clearly defined. The suitable origins of replication for mammalian vectors are normally from eukaryotic viruses. Preferred eukaryotic on include, but are not limited to, SV40 ori, EBV ori, or HSV ori.

As used herein, a “promoter” is a DNA region capable under certain conditions of binding RNA polymerase and initiating transcription of a coding region located downstream (in the 3′ direction) from the promoter. It can be constitutive or inducible. In general, the promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site, as well as protein binding domains responsible for the binding of RNA polymerase. Eukaryotic promoters will often, but not always, contain “TATA” boxes and “CAT” boxes.

The choice of promoters will largely depend on the host cells in which the vector is introduced. For prokaryotic cells, a variety of robust promoters are known in the art. Preferred promoters are lac promoter, Trc promoter, T7 promoter and pBAD promoter. Normally, to obtain expression of exogenous sequence in multiple species, the prokaryotic promoter can be placed immediately after the eukaryotic promoter, or inside an intron sequence downstream of the eukaryotic promoter.

Suitable promoter sequences for eukaryotic cells include the promoters for 3-phosphoglycerate kinase, or other glycolytic enzymes, such as enolase, glyceraldehyde-3phosphate dehydrogenase, hexokinase, pyruvate decarboxylase, phosphofructokinase, glucose-6phosphate isomerase, 3-phosphogly cerate mutase, pyruvate kinase, triosephosphate isomerase, phosphoglucose isomerase, and glucokinase. Other promoters, which have the additional advantage of transcription controlled by growth conditions, are the promoter regions for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Preferred promoters for mammalian cells are SV40 promoter, CMV promoter, b-actin promoter and their hybrids. Preferred promoters for yeast cell include but is not limited to GAL 10, GAL I, TEFI in S. cerevisiae, and GAP, AOX1 in P. pastoris.

In constructing the subject vectors, the termination sequences associated with the exogenous sequence are also inserted into the 3′ end of the sequence desired to be transcribed to provide polyadenylation of the mRNA and/or transcriptional termination signal. The terminator sequence preferably contains one or more transcriptional termination sequences (such as polyadenylation sequences) and may also be lengthened by the inclusion of additional DNA sequence so as to further disrupt transcriptional read-through. Preferred terminator sequences (or termination sites) of the present invention have a gene that is followed by a transcription termination sequence, either its own termination sequence or a heterologous termination sequence. Examples of such termination sequences include stop codons coupled to various yeast transcriptional termination sequences or mammalian polyadenylation sequences that are known in the art and are widely available. Where the terminator comprises a gene, it can be advantageous to use a gene that encodes a detectable or selectable marker; thereby providing a means by which the presence and/or absence of the terminator sequence (and therefore the corresponding inactivation and/or activation of the transcription unit) can be detected and/or selected.

In addition to the above-described elements, the vectors may contain a selectable marker (for example, a gene encoding a protein necessary for the survival or growth of a host cell transformed with the vector), although such a marker gene can be carried on another polynucleotide sequence co-introduced into the host cell. Only those host cells into which a selectable gene has been introduced will survive and/or grow under selective conditions. Typical selection genes encode protein(s) that (a) confer resistance to antibiotics or other toxins, e.g., ampicillin, kanamycin, neomycin, zeocin, G418, methotrexate, etc.; (b) complement auxotrophic deficiencies; or (c) supply critical nutrients not available from complex media. The choice of the proper marker gene will depend on the host cell, and appropriate genes for different hosts are known in the art.

In one embodiment, the expression vector is a shuttle vector, capable of replicating in at least two unrelated host systems. To facilitate such replication, the vector generally contains at least two origins of replication, one effective in each host system. Typically, shuttle vectors are capable of replicating in a eukaryotic host system and a prokaryotic host system. This enables detection of protein expression in the eukaryotic host (the expression cell type) and amplification of the vector in the prokaryotic host (the amplification cell type). Preferably, one origin of replication is derived from SV40 or 2u and one is derived from pUC, although any suitable origin known in the art may be used provided it directs replication of the vector. Where the vector is a shuttle vector, the vector preferably contains at least two selectable markers, one for the expression cell type and one for the amplification cell type. Any selectable marker known in the art or those described herein may be used provided it functions in the expression system being utilized.

The vectors encompassed by the invention can be obtained using recombinant cloning methods and/or by chemical synthesis. A vast number of recombinant cloning techniques such as PCR, restriction endonuclease digestion and ligation are well known in the art, and need not be described in detail herein. One of skill in the art can also use the sequence data provided herein or that in the public or proprietary databases to obtain a desired vector by any synthetic means available in the art. Additionally, using well-known restriction and ligation techniques, appropriate sequences can be excised from various DNA sources and integrated in operative relationship with the exogenous sequences to be expressed in accordance with the present invention.

Depending on the specific vector used for expressing the fusion polypeptide, various known cells or cell lines can be employed in the practice of the invention. The host cell can be any cell into which recombinant vectors carrying a fusion of the invention may be introduced and the vectors are permitted to drive the expression of the fusion polypeptide is useful for the invention. It may be prokaryotic, such as any of a number of bacterial strains, or may be eukaryotic, such as yeast or other fungal cells, insect or amphibian cells, or mammalian cells including, for example, rodent, simian or human cells. Cells expressing the fusion polypeptides of the invention may be primary cultured cells or may be an established cell line. Thus, in addition to the cell lines exemplified herein (e.g., CHO cells), a number of other host cell lines capable well known in the art may also be used in the practice of the invention. These include, e.g., various Cos cell lines, HeLa cells, HEK293, AtT20, BV2, and N18 cells, myeloma cell lines, transformed B-cells and hybridomas.

The use of mammalian tissue cell culture to express polypeptides is discussed generally in, e.g., Winnacker, From Genes to Clones, VCH Publishers, N.Y., N.Y., 1987. The fusion polypeptide-expressing vectors may be introduced to the selected host cells by any of a number of suitable methods known to those skilled in the art. For the introduction of fusion polypeptide-encoding vectors to mammalian cells, the method used will depend upon the form of the vector. For plasmid vectors, DNA encoding the fusion polypeptide sequences may be introduced by any of a number of transfection methods, including, for example, lipid-mediated transfection (“lipofection”), DEAE-dextran-mediated transfection, electroporation or calcium phosphate precipitation. These methods are detailed, for example, in Brent et al, supra. Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™ (Stratagene) kits are available. Other companies offering reagents and methods for lipofection include Bio-Rad Laboratories, CLONTECH, Glen Research, Life Technologies, JBL Scientific, MBI Fermentas, PanVera, Promega, Quantum Biotechnologies, Sigma-Aldrich, and Wako Chemicals USA.

For long-term, high-yield production of recombinant fusion polypeptides, stable expression is preferred. Rather than using expression vectors which contain viral origins of replication, host cells can be transformed with the fusion polypeptide encoding sequences controlled by appropriate expression control elements (e.g., promoter, enhancer, sequences, transcription terminators, polyadenylation sites, etc.), and selectable markers. The selectable marker in the recombinant vector confers resistance to the selection and allows cells to stably integrate the vector into their chromosomes. Commonly used selectable markers include neo, which confers resistance to the aminoglycoside G-418 (Colberre-Garapin, et al, J. Mol. Biol., 150: 1, 1981); and hygro, which confers resistance to hygromycin (Santerre et al, Gene, 30: 147, 1984). Through appropriate selections, the transfected cells can contain integrated copies of the fusion polypeptide encoding sequence.

### Compositions

One aspect of the present disclosure provides an immunogenic composition comprising, consisting of, or consist essentially of the immunogenic conjugate described herein.

Another aspect of the present disclosure provides a pharmaceutical composition comprising, consisting of, or consist essentially of the immunogenic composition described herein and a pharmaceutically acceptable carrier.

In some embodiments, the immunogenic conjugate comprises from N-terminus to C-Terminus: a polypeptide of an antigen of interest, a recombined tether/capture sequence, a glycine linker, and a self-assembly KARI nanoparticle sequence. In some embodiments, the immunogenic conjugate comprises from N-terminus to C-Terminus: a polypeptide of an antigen of interest, a trimerization domain, a recombined tether/capture sequence, a glycine linker, and a self-assembly KARI nanoparticle sequence. In some embodiments, the immunogenic conjugate comprises from N-terminus to C-Terminus: a polypeptide of an antigen of interest, a trimerization domain, a recombined tether/capture sequence, a glycine linker, a self-assembly KARI nanoparticle sequence, and a T-cell epitope.

In some embodiments, the immunogenic conjugate comprises from N-terminus to C-Terminus: a polypeptide of an antigen of interest, a recombined tether/capture sequence, a glycine linker, a self-assembly KARI nanoparticle sequence, and a T-cell epitope.

In some embodiments, the immunogenic conjugate comprises from N-terminus to C-Terminus: a polypeptide of an antigen of interest of SEQ ID NO: 11, a glycine linker of SEQ ID NOs: 5, 9, or 16-20, and a self-assembly KARI nanoparticle sequence of SEQ ID NO: 1 or 2. In some embodiments, the immunogenic conjugate comprises from N-terminus to C-Terminus: a polypeptide of an antigen of interest of SEQ ID NO: 11, a trimerization domain of SEQ ID NO: 21 or 22, a glycine linker of SEQ ID NOs: 5, 9, or 16-20, and a self-assembly KARI nanoparticle sequence of SEQ ID NO: 1 or 2. In some embodiments, the immunogenic conjugate comprises from N-terminus to C-Terminus: a polypeptide of an antigen of interest of SEQ ID NO: 11, a trimerization domain of SEQ ID NO: 21 or 22, a glycine linker a glycine linker f SEQ ID NOs: 5, 9, or 16-20, a self-assembly KARI nanoparticle sequence of SEQ ID NO: 1 or 2, and a T-cell epitope of SEQ ID NO: 22-27. In some embodiments, the immunogenic conjugate comprises from N-terminus to C-Terminus: a polypeptide of an antigen of interest of SEQ ID NO: 11, a glycine linker of SEQ ID NOs: 5, 9, or 16-20, a self-assembly KARI nanoparticle sequence of SEQ ID NO: 1 or 2, and a T-cell epitope of SEQ ID NO: 22-27.

In some embodiments of the immunogenic composition described herein, the immunogenic conjugate or recombinant KARI protein further comprises a purification tag. In some embodiments, the purification tag is operably linked to a C-terminus or an N-terminus of the recombinant KARI.

In another aspect, the present disclosure provides immunogenic compositions comprising a disclosed recombinant KARI protein or a self-assembling nanoparticle described herein, and a pharmaceutically acceptable carrier are also provided. Such compositions can be administered to a subject by a variety of modes, for example, by an intranasal route. Standard methods for preparing administrable immunogenic compositions are described, for example, in such publications as Remingtons Pharmaceutical Sciences, 19^{th} Ed., Mack Publishing Company, Easton, Pa., 1995.

### Formulations

The immunogenic compositions can be formulated for administration to a subject by a variety of administration modes, including mucosal administration modes such as by oral, rectal, intranasal, intrapulmonary, or transdermal delivery, or by topical delivery to other surfaces, and non-mucosal routes, including by intramuscular, subcutaneous, intravenous, intra-arterial, intra-articular, intraperitoneal, or parenteral routes.

Potential carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides, or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

The immunogenic composition can contain a bacteriostat to prevent or minimize degradation during storage, including but not limited to effective concentrations (usually 1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The immunogenic composition can contain as pharmaceutically acceptable vehicles substances as required to approximate physiological conditions, such as pH adjusting and buffering agents, tonicity adjusting agents, wetting agents, and the like, for example, sodium acetate, sodium lactate, sodium chloride, potassium chloride, calcium chloride, sorbitan monolaurate, and triethanolamine oleate.

In certain embodiments, the recombinant KARI proteins or self-assembling nanoparticles can be administered in a time-release formulation, for example in a immunogenic composition that includes a slow release polymer. These compositions can be prepared with vehicles that will protect against rapid release, for example a controlled release vehicle such as a polymer, microencapsulated delivery system or bioadhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent, and which is capable of incorporating the vaccine and/or other biologically active agent. Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

The immunogenic composition can optionally include an adjuvant to enhance the immune response of the host. Suitable adjuvants are, for example, toll-like receptor agonists, alum, AlPO4, alhydrogel, Lipid-A and derivatives or variants thereof, oil-emulsions, saponins, neutral liposomes, liposomes containing the recombinant virus, and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, Ind.) and IL-12 (Genetics Institute, Cambridge, Mass.), among many other suitable adjuvants well known in the art, may be used as an adjuvant (Newman et al., 1998, Critical Reviews in Therapeutic Drug Carrier Systems 15:89-142). These adjuvants have the advantage in that they help to stimulate the immune system in a non-specific way, thus enhancing the immune response to a pharmaceutical product.

The compositions of the present disclosure can additionally include components to provide sustained release and/or comfort. Such components include high molecular weight, anionic mucomimetic polymers, gelling polysaccharides and finely divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., G&o Pharm. Res. 12:857-863, 1995); or, as microspheres for oral administration (see e.g., Eyles, J. Pharm. Pharmacol. 49:669-674, 1997). In embodiments, the formulations of the compositions of the present invention can be delivered using liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See e.g., A1-Muhammed, J. Microencapsul. 13:293-306, 1996; Chonn, Curr. Opin. Biotechnol. 6:698-708, 1995; Ostro, Am. J. Hosp. Pharm. 46: 1576-1587, 1989). Dosage

In some embodiments, the immunogenic composition can be provided in unit dosage form for use to induce an immune response in a subject, for example, to prevent or inhibit HIV-1 or influenza infection in the subject. A unit dosage form contains a suitable single preselected dosage for administration to a subject, or suitable marked or measured multiples of two or more preselected unit dosages, and/or a metering mechanism for administering the unit dose or multiples thereof.

The immunogenic composition typically contains an effective amount of a disclosed recombinant KARI protein or self-assembling nanoparticles and can be prepared by conventional techniques. Preparation of immunogenic compositions, including those for administration to human subjects, is generally described in Pharmaceutical Biotechnology, Vol. 61 Vaccine Design—the subunit and adjuvant approach, edited by Powell and Newman, Plenum Press, 1995. New Trends and Developments in Vaccines, edited by Voller et al., University Park Press, Baltimore, Md., U.S.A. 1978. Typically, the amount of antigen in each dose of the immunogenic composition is selected as an amount that induces an immune response without significant adverse side effects.

The amount of the disclosed recombinant KARI protein or self-assembling nanoparticles included in the therapeutic composition can vary depending upon the specific antigen employed, the route and protocol of administration, and the target population, for example. For protein therapeutics, typically, each human dose will comprise 1-1000 μg of protein, such as from about 1 μg to about 100 μg, for example, from about 1 μg to about 50 μg, such as about 1 μg, about 2 μg, about 5 μg, about 10 μg, about 15 μg, about 20 μg, about 25 μg, about 30 μg, about 40 μg, or about 50 μg. The amount utilized in an immunogenic composition is selected based on the subject population (e.g., infant or elderly). An optimal amount for a particular composition can be ascertained by standard studies involving observation of antibody titers and other responses in subjects. It is understood that a therapeutically effective amount of a disclosed self-assembling KARI nanoparticle comprises an amount that is ineffective at eliciting an immune response by administration of a single dose, but that is effective upon administration of multiple dosages, for example in a prime-boost administration protocol.

### Methods of Treatment

One aspect of the present disclosure provides a method for preventing or treating a disease in a subject, comprising administering to the subject a pharmaceutically effective amount of the immunogenic composition or the pharmaceutical composition described herein.

Another aspect of the present disclosure provides a method for generating an immune response to a protein of interest in a subject, comprising administering to the subject an effective amount of the immunogenic conjugate described herein, the immunogenic composition described herein, or a composition described herein to generate the immune response.

The disclosed self-assembling KARI nanoparticles can be administered to a subject to elicit an immune response to protein of interest included on the self-assembling KARI nanoparticle in the subject. Upon immunization, the subject responds by producing antibodies specific for the protein of interest comprised in the self-assembling KARI nanoparticle. In addition, innate and cell-mediated immune responses are induced, which can provide for example antiviral effectors as well as regulating the immune response. The immune response can be a protective immune response, for example a response that prevents or reduces subsequent infection with a virus including the trimeric antigen. The immune response can be a therapeutic immune response, for example a response that treats or inhibits current infection with a virus including the trimeric antigen and illnesses associated therewith.

Typical subjects intended for treatment with the compositions and methods of the present disclosure include mammals, such as humans, non-human primates, as well as other animals. To identify subjects for prophylaxis or treatment according to the methods of the disclosure, accepted screening methods are employed to determine risk factors associated with a targeted or suspected disease of condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods. These and other routine methods allow the clinician to select patients in need of therapy using the methods and immunogenic compositions of the disclosure. In accordance with these methods and principles, a self-assembling KARI nanoparticle and/or other biologically active agent can be administered according to the teachings herein as an independent prophylaxis or treatment program, or as a follow-up, adjunct or coordinate treatment regimen to other treatments.

In some embodiments, a subject is selected for treatment that has, or is at risk for developing, a bacterial, viral, or fungal infection, for example because of exposure or the possibility of exposure to a bacterium, a virus or a fungus. Such a subject can then be administered an effective amount of a self-assembling KARI nanoparticle described herein including a protein of interest, such as a known antigen to induce an immune response to the antigen. The immune response can neutralize autologous virus (e.g., the strains comprising the antigens on the nanoparticle) or heterologous virus (e.g., strains other than the strain comprising the antigen on the nanoparticle).

In some embodiments, the immune response treats or inhibits an infection or a disease progression in the subject. In some embodiments, the administration of the immunogenic conjugate or immunogenic composition to the subject primes a protective immune response to an infection or a condition triggered by the protein of interest in the subject.

In some such embodiments, the immune response inhibits subsequent infection (for example, as measured by infection of cells, or by number or percentage of subjects infected by a virus, a bacterium, or a fungus) by a desired amount, for example by at least 10%, at least 20%, at least 50%, at least 60%, at least 70%, at least 80%, at least 90%, at least 95%, at least 98%, or even at least 100% (prevention of detectable infection), as compared to a suitable control.

In some embodiments, a subject is selected for treatment that has, or is at risk for developing, an infection, for example because of exposure or the possibility of exposure to influenza. Such a subject can then be administered an effective amount of a self-assembling KARI nanoparticle.

In some embodiments, a subject is selected for treatment that has, or is at risk for developing, a viral, a bacterial or fungal infection. In some embodiments, the subject is at risk of developing a viral infection. In that embodiment, such a subject can then be administered an effective amount of a self-assembling KARI nanoparticle described herein including a viral antigen selected from a coronavirus. In some embodiments, the coronavirus SARS-CoV-1, MERS-CoV, 229E (alpha), NL63 (alpha), OC43 (beta), HKU1 (beta), or SARS-CoV-2; or

In some embodiments, the coronavirus is a SARS-CoV-2 or a variant thereof selected from B.1.1.7, B.1.1.7 with E484K, B.1.135, B.1.351, P.1, B.1.427, D614G, B.1.1351, B.1.429, Lambda (C.37), or Mu (B.1.621).

In those embodiments, the viral antigen is selected from a coronavirus spike protein; a mutant coronavirus spike protein; a mutant coronavirus spike protein comprising 1, 2, 3, 4, or 5 proline mutations selected from F817P, A892P, A899P, A942P, P986K, K986P, V987P, and P987V; and/or a mutant coronavirus spike protein comprising at least one additional disulfide bond selected from F43C-G566C, G413C-P987C, Y707C-T883C, G1035C-V1040C, A701C-Q787C, G667C-L864C, V382C-R983C, and I712C-I816C.

In those embodiments, the mutant coronavirus spike protein comprises the amino acid sequence of SEQ ID NO: 11 or an amino acid sequence having at least about 70%, at least about 75%, at least about 80%, at least about 85%, at least about 90%, at least about 95%, at least about 97%, or at least about 99% identity to the amino acid sequence of SEQ ID NO:11 (across the full length of the amino acid sequence). In some embodiments, the mutant coronavirus spike protein comprises the amino acid sequence of a spike protein disclosed in, for example, WO 2022/087255A2, which is incorporated by reference in its entirety.

In some embodiments, the immune response induced by the self-assembling nanoparticle can neutralize autologous virus (e.g., the antigen from the specific SARS-CoV-2 strain included on the KARI nanoparticle) or heterologous virus (e.g., strains other than the strain from which the antigen on the nanoparticle was derived). In some such embodiments, the immune response inhibits subsequent SARS-CoV-2 infection (for example, as measured by infection of cells, or by number or percentage of subjects infected by SARS-CoV-2) by a desired amount, for example by at least 10%, at least 20%, at least 50%, at least 60%, at least 70%, at least 80%, at least 90%, at least 95%, at least 98%, or even at least 100% (prevention of detectable SARS-CoV-2 infection), as compared to a suitable control.

The immunogenic composition may be administered by any suitable method, including but not limited to, via injection, aerosol delivery, nasal spray, nasal droplets, oral inoculation, or topical application.

An effective amount of the self-assembling KARI nanoparticle and/or other biologically active agent is administered to a subject in need of such treatment for a time and under conditions sufficient to prevent, inhibit, and/or ameliorate a selected disease or condition or one or more symptom(s) thereof.

The self-assembling KARI nanoparticle can be administered to the subject in a single bolus delivery, via continuous delivery (for example, continuous transdermal, mucosal, or intravenous delivery) over an extended time period, or in a repeated administration protocol (for example, by an hourly, daily or weekly, repeated administration protocol). The effective amount of the KARI nanoparticle can be provided as repeated doses within a prolonged prophylaxis or treatment regimen that will yield clinically significant results to alleviate one or more symptoms or detectable conditions associated with a targeted disease or condition as set forth herein.

The administration of an effective amount of a self-assembling KARI nanoparticle of the disclosure can be for either prophylactic or therapeutic purpose. When provided prophylactically, the self-assembling KARI nanoparticle is provided in advance of any symptom, for example in advance of infection, such as in the form of a yearly flu shot. The prophylactic administration of the self-assembling KARI nanoparticle serves to prevent or ameliorate any subsequent infection. When provided therapeutically, the self-assembling KARI nanoparticle is provided at (or shortly after) the onset of a symptom of disease or infection. Thus, when used to prevent or treat a viral infection (such as coronavirus, SARS-CoV-1, MERS-CoV, 229E (alpha), NL63 (alpha), OC43 (beta), HKU1 (beta), SARS-CoV-2, HIV-1, influenza, RSV, and/or MPV infection), the KARI nanoparticle of the disclosure can be provided prior to the anticipated exposure to virus so as to attenuate the anticipated severity, duration or extent of an infection and/or associated disease symptoms, after exposure or suspected exposure to the virus, or after the actual initiation of an infection.

Determination of effective dosages is typically based on animal model studies followed up by human clinical trials and is guided by administration protocols that significantly reduce the occurrence or severity of targeted disease symptoms or conditions in the subject. Suitable models in this regard include, for example, murine, rat, porcine, feline, ferret, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of the KARI nanoparticle (for example, amounts that are effective to elicit a desired immune response or alleviate one or more symptoms of a targeted disease).

The actual dosage of the KARI nanoparticle will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the vaccine for eliciting the desired activity or biological response in the subject. Dosage regimens can be adjusted to provide an optimum prophylactic or therapeutic response. As described above in the forgoing listing of terms, a therapeutically effective amount is also one in which any toxic or detrimental side effects of the KARI nanoparticle and/or other biologically active agent is outweighed in clinical terms by therapeutically beneficial effects. A non-limiting range for a therapeutically effective amount of a self-assembling nanoparticle and/or other biologically active agent within the methods and formulations of the disclosure is about 0.01 mg/kg body weight to about 10 mg/kg body weight, such as about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.5 mg/kg, about 2 mg/kg, about 2.5 mg/kg, about 3 mg/kg, about 4 mg/kg, about 5 mg/kg, or about 10 mg/kg, for example 0.01 mg/kg to about 1 mg/kg body weight, about 0.05 mg/kg to about 5 mg/kg body weight, about 0.2 mg/kg to about 2 mg/kg body weight, or about 1.0 mg/kg to about 10 mg/kg body weight. In some embodiments, the dosage includes a set amount of a disclosed self-assembling KARI nanoparticle, such as from about 1-300 μg, for example, a dosage of about 10-300 μg, about 60 μg, about 1, 2, 3, 4, 5, 6, 7, 8, 9, 10, 15, 20, 30, 40, 50, 60, 70, 80, 90, 100, 150, 200, 250, or about 300 μg. As used herein with reference to a concentration or amount, “about” refers to +/−5%. Therefore, “about 100 μg” refers to 95-105 μg.

Upon administration of an effective amount of a disclosed self-assembling KARI nanoparticle (for example, via injection, aerosol, oral, topical or other route), the immune system of the subject typically responds to the self-assembling KARI nanoparticle by producing antibodies specific for the trimeric antigens on the self-assembling KARI nanoparticle. Such a response signifies that an effective amount of the self-assembling KARI nanoparticle was delivered. An effective amount can be achieved by single or multiple administrations (including, for example, multiple administrations per day), daily, or weekly administrations. For each subject, specific dosage regimens can be evaluated and adjusted over time according to the individual need and professional judgment of the person administering or supervising the administration of the vaccine. In some embodiments, the antibody response of a subject administered the compositions of the disclosure will be determined in the context of evaluating effective dosages/immunization protocols. In most instances it will be sufficient to assess the antibody titer in serum or plasma obtained from the subject. Decisions as to whether to administer booster inoculations and/or to change the amount of the composition administered to the individual can be at least partially based on the antibody titer level. The antibody titer level can be based on, for example, an immunobinding assay which measures the concentration of antibodies in the serum that bind to a specific antigen, for example, influenza HA protein.

An immunogenic composition including one or more of the disclosed self-assembling KARI nanoparticles can be used in coordinate (or prime-boost) vaccination protocols or combinatorial formulations. In certain embodiments, novel combinatorial immunogenic compositions and coordinate immunization protocols employ separate r self-assembling KARI nanoparticle or formulations, each directed toward eliciting an anti-viral immune response, such as an immune response to HIV-1 Env proteins. Separate immunogenic compositions that elicit the anti-viral immune response can be combined in a polyvalent immunogenic composition administered to a subject in a single immunization step, or they can be administered separately (in monovalent immunogenic compositions) in a coordinate (or prime boost) immunization protocol.

There can be several boosts, and each boost can be a different disclosed immunogen. In some examples that the boost may be the same immunogen as another boost, or the prime. The prime and boost can be administered as a single dose or multiple doses, for example two doses, three doses, four doses, five doses, six doses or more can be administered to a subject over days, weeks or months. Multiple boosts can also be given, such one to five (e.g., 1, 2, 3, 4 or 5 boosts), or more. Different dosages can be used in a series of sequential immunizations. For example a relatively large dose in a primary immunization and then a boost with relatively smaller doses.

In some embodiments, the boost can be administered about two, about three to eight, or about four, weeks following the prime, or about several months after the prime. In some embodiments, the boost can be administered about 5, about 6, about 7, about 8, about 10, about 12, about 18, about 24, months after the prime, or more or less time after the prime. Periodic additional boosts can also be used at appropriate time points to enhance the subject's “immune memory.”

The adequacy of the vaccination parameters chosen, e.g., formulation, dose, regimen and the like, can be determined by taking aliquots of serum from the subject and assaying antibody titers and/or neutralizing activity during the course of the immunization program. To assess neutralization activity, following immunization of a subject, serum can be collected from the subject at appropriate time points, frozen, and stored for neutralization testing. Methods to assay for neutralization activity are known and are further described herein, and include, but are not limited to, plaque reduction neutralization (PRNT) assays, microneutralization assays, flow cytometry-based assays, single-cycle infection assays. In some embodiments, the serum neutralization activity can be assayed using a panel of pseudoviruses. In addition, the clinical condition of the subject can be monitored for the desired effect. If such monitoring indicates that vaccination is sub-optimal, the subject can be boosted with an additional dose of immunogenic composition, and the vaccination parameters can be modified in a fashion expected to potentiate the immune response. Thus, for example, the dose of the disclosed immunogen can be increased or the route of administration can be changed.

### Using Self-Assembly Nanoparticles

The antigenicity and structural integrity of the protein of interest (e.g., SARS-CoV-2 nanoparticle) can be readily analyzed via standard assays, e.g., antibody binding assays and negative-stain electron microscopy (EM). As exemplified herein, the fusion molecules can all self-assemble into nanoparticles that display immunogenic epitopes of theSARS-CoV-2 Spike trimer. By eliciting robust immunogen-specific antibodies, the nanoparticle described herein are useful for vaccinating individuals against a broad range of viruses (e.g., SARS-CoV-2, HIV-1, Ebola, Lassa, and HCV viruses) as described herein.

The effectiveness of a prophylactic vaccine is determined by the generation of a long-lasting T-cell-dependent IgG antibody T-cells by antigen presenting cells (APC) followed by the activation of B-cells by antigen and a specialized T helper cell (the T Follicular helper cell; TFH cell) response. Generating this response involves the activation of APCs phagocytize, process, and present antigens to T-cells. When APCs recognize, they become activated and differentiate into T helper cells. Naive B-cells require two signals to mature into high-affinity IgG plasma cells and receive a first signal from the crosslinking of multiple B-cell receptors (BCR) with an antigen that has directly entered the lymph node or was presented by APCs, followed by endocytosis of the receptor and antigen, and then presentation of the processed antigen on the surface. The second signal is delivered upon recognition of the B-cell-presented antigen with a TFH cell, which activates the B-cell to mature into an IgG-producing plasma cell.

Two characteristics of the KARI nanoparticle platforms disclosed herein contribute to generating the B-cell IgG response: (1) the attachment of the antigen to a larger scaffold, which improves APC uptake and retention in lymph follicles and (2) the repetitive array of antigens, which enables efficient binding and activation of multiple B-cell receptors. Attachment of antigens on particles increases the overall particulate size into an optimal size range for efficient uptake by APCs, which allows for greater presentation of antigen by APCs to activate T-helper cells. Larger particles are also efficiently opsonized with complement. Opsonization promotes binding to the surface of follicular dendritic cells (FDCs), elongating retention in lymph follicles, and enhancing antigen presentation to B-cells. Particles displaying numerous antigens can then facilitate B-cell activation through efficient crosslinking with multiple BCRs. High-density conjugation of an antigen to a VLP activated a specific IgG antibody response, while low-density conjugation did not (despite increased antigen quantity), suggesting an effect outside the total amount of antigen.

Accordingly, the self-assembling KARI nanoparticles of the present disclosure will enhance the immunogenicity of and the affinity and maturation of the elicited B cells. In some embodiments, the nanoparticles disclosed herein are used as an antigen and/or drug delivery vehicle to target antigen presenting cells (APCs) such as e.g., dendritic cells (DCs) in lymph nodes. The ability to deliver antigen and/or drugs and/or danger signals to DCs in lymph nodes is a useful approach to immunotherapy. In some embodiments, the KARI nanoparticles of the present invention is used in a method of targeting or delivering antigenic proteins and polypeptides and/or antigen-encoding nucleic acids to lymph node DCs and other APCs. Since DCs are critically involved in initiating cell-mediated immunity by antigen presentation to T cells, this delivery approach can be utilized for several vaccine and immunotherapy applications.

In some embodiments, the KARI nanoparticles or immunogenic conjugate described herein is useful as diagnostic tool (e.g., imaging), research tools (e.g., as sold in ALDRICH catalogs or for visualization using microscopes), or in vitro drug delivery or visualization (e.g., APC and/or DC and/or macrophage uptake in vitro of drugs or imaging agents.

The novel nanoparticle platform of the present disclosure (e.g., KARI) can also be used to display antibodies or nanobodies to improve their avidity. As such, the KARI nanoparticle platform can be useful for cancer immunotherapy. Furthermore, the KARI nanoparticle platform can be useful for identifying and characterizing T-cell epitopes and activity. In some embodiments, the protein of interest fused to the nanoparticle is an antibody, an antibody fragment, a single chain antibody, scFv, scFab, single domain antibody, a protein scaffolds, an enzyme, a hormone, or an interleukin. In another embodiment, the protein of interest is a diagnostic agent. In another embodiment, the protein of interest is an imaging agent.

Accordingly, one aspect of the present disclosure provides a diagnostic agent comprising the self-assembly nanoparticle, or the immunogenic conjugate described herein. In some embodiments, the diagnostic agent identifies one or more T cell epitopes and/or antigens in a biological sample. In some embodiments, the biological sample is from a mammalian subject. In some embodiments, the diagnostic agent measures T cell response and/or activity in a biological sample. Another aspect of the present disclosure provides, the use of the self-assembly nanoparticle, or the immunogenic conjugate described herein as a diagnostic agent. In some embodiments, the diagnostic agent detects one or more pathogens in a biological sample, e.g., by binding to one or more antibodies generated by the subject. In some embodiments, the KARI nanoparticle or the immunogenic conjugate may be used as an antigen in immunoassays such as ELISA (enzyme-linked immunosorbent assay) and immunoblot for detecting antibodies specific to one or more proteins or peptides conjugated to the nanoparticle. In some embodiments, the KARI nanoparticle or the immunogenic conjugate may be used in a microarray format. For example, the KARI nanoparticle or the immunogenic conjugate comprising different proteins or peptides may be used to detect a range of specific antibodies.

Another aspect of the present disclosure provides a method of cell sorting comprising, consisting of, consisting essentially of introducing the self-assembly nanoparticle, or the immunogenic conjugate described herein into a cell sorting apparatus comprising a population of cells; allowing the self-assembly nanoparticle or the immunogenic conjugate to bond with specific type of cells, and sorting cells based on said bonding.

Another aspect of the present disclosure provides a method of imaging a target material comprising, consisting of, consisting essentially: introducing the self-assembly nanoparticle of as disclosed herein, or the immunogenic conjugate as disclosed herein into a medium containing said target material; allowing the self-assembly nanoparticle or the immunogenic conjugate to bond with the target material; and obtaining an image of the target based on bonding between the self-assembly nanoparticle or the immunogenic conjugate and the target.

In some embodiments, the protein of interest is an antibody, an antibody fragment, a single chain antibody, scFv, scFab, single domain antibody, a protein scaffolds, an enzyme, a hormone, or an interleukin. In another embodiment, the protein of interest is a diagnostic agent. In another embodiment, the protein of interest is an imaging agent.

### Kits

One aspect of the present disclosure provides a kit comprising one or more of the following an immunogenic conjugate, a self-assembling KARI nanoparticle, a vector or a combination of vectors comprising the immunogenic conjugate or self-assembling KARI nanoparticle, composition or nucleic acids encoding of the immunogenic conjugate or self-assembling KARI nanoparticle as described herein.

In some embodiments, the kit is for therapeutic/prophylactic use. In some embodiments, the kit comprises a pharmaceutically acceptable carrier. In some embodiments, the kit of the present disclosure is packaged with instructions for use in a method or use of the invention as described herein.

### EXAMPLES

These examples are provided for illustrative purposes only and not to limit the scope of the claims provided herein.

The examples shown below describe the generation of a self-assembling KARI nanoparticle and its usage as platform to display 12 copies of antigens and/or antibodies to improve the recognition of those antigens by the immune system.

### Example 1: KARI, a Novel Protein for Nanoparticle Formation—

This example provides a recombinant archaeal ketol-acid reductoisomerase (KARI) protein that was used to generate self-assembling nanoparticles.

An archaeal ketol-acid reductoisomerase (KARI) was tested as a novel protein nanoparticle. The recently solved crystal structure of the KARI protein from Sulfolobus solfataricus (Sso) showed that KARI oligomerized in a 12-mer nanoparticle with a compact core formed by long helix-alpha and that the KARI is thermostable. FIGS. 1A-B. As shown in FIG. 1B, the melting temperature of the KARI nanoparticle is higher than 85° C.

This nanoparticle can be expressed in E. coli linked with different versions of SpyCatcher, SpyTag or the sortase substrate tag (GGGGG; SEQ ID NO: 8) in the N-terminal to optimize the SpyCatcher/SpyTag coupling. Each monomer of the KARI protein (recombinant KARI protein of SEQ ID NO: 1 or 2) can be genetically fused to the SpyCatcher or the sortase A recognition domain. In that configuration, 12 SpyCatchers/sortase will be attached on the external part of the nanoparticle. FIG. 3A-B.

The sequence of the KARI nanoparticle (wild-type) is set forth in SEQ ID NO: 1.

MDKTVLDANLDPLKGKTIGVIGYGNQGRVQATIMRENGLNVIVGNVKDKYYEL
AKKEGFEVYEIDEAVRRSDVALLLIPDEVMKEVYEKKIAPVLQGKKEFVLDFASGYNVA
FGLIRPPKSVDTIMVAPRMVGEGIMDLHKQGKGYPVLLGVKQDASGKAWDYAKAIAK
GIGAIPGGIAVISSFEEEALLDLMSEHTWVPILFGAIKACYDIAVKEYGVSPEAALLEFYAS
GELAEIARLIAEEGIFNQMVHHSTTSQYGTLTRMFKYYDVVRRIVENEAKYIWDGSFAK
EWSLEQQAGYPVFYRLWELATQSEMAKAEKELYKLLGRKVKND

In addition, a KARI variant lacking the first 12 amino acids in the N-terminus was also generated. The amino acid sequence of a KARI nanoparticle lacking the 12 first amino acids is set forth in SEQ ID NO: 2. Either the wild-type or the N-terminus variant was cloned in an expression vector, pET plasmid, for expression in bacteria.

For the expression and coupling with the antigen, the KARI gene was flanked by a SpyCatcher in the 5′ of the gene, and an HRV-C3 protease cleavage site followed by double twin strep tag for purification, both placed in the 3′ of the gene. The recombinant KARI protein (genetically fused protein) produced monomers of the nanoparticle with the SpyCatcher on the N-terminal and strep tags on the C-terminal. The strep tag was removed by incubating the protein with an HRV-C3 protease overnight at room temperature.

The final sequence for the SpyCatcher/KARI_NP (recombinant KARI protein; or KARI nanoparticle monomer) comprised the amino acid sequence set forth in SEQ ID NO: 3 (SpyCatcher001-GS linker—KARI—Purification tats):

MSATHIKFSKRDEDGKELAGATMELRDSSGKTISTWISDGQVKDFYLYPGK
YTFVETAAPDGYEVATAITFTVNEQGQVTVNGKATKGDAHIGSLKGKTIGVIGYGNQ
GRVQATIMRENGLNVIVGNVKDKYYELAKKEGFEVYEIDEAVRRSDVALLLIPDEVMKEVYEK
KIAPVLQGKKEFVLDFASGYNVAFGLIRPPKSVDTIMVAPRMVGEGIMDLHKQGKGYPVLLG
VKQDASGKAWDYAKAIAKGIGAIPGGIAVISSFEEEALLDLMSEHTWVPILFGAIKACYDIAVK
EYGVSPEAALLEFYASGELAEIARLIAEEGIFNQMVHHSTTSQYGTLTRMFKYYDVVRRIVENEA
KYIWDGSFAKEWSLEQQAGYPVFYRLWELATQSEMAKAEKELYKLLGRKVKNDLEVLFQGP
WSHPQFEKGGGSGGGSGGGSWSHPQFEK

The final sequence for the Sortase/KARI NP (recombinant KARI protein; or KARI nanoparticle monomer) comprise the amino acid sequence set forth in SEQ ID NO: 7 (Sortase-GS linker—KARI—Purification taas):

GGGGGSGGSGSLKGKTIGVIGYGNQGRVQATIMRENGLNVIVGNVKDKYYELAKKE
GFEVYEIDEAVRRSDVALLLIPDEVMKEVYEKKIAPVLQGKKEFVLDFASGYNVAFGLIRPPKS
VDTIMVAPRMVGEGIMDLHKQGKGYPVLLGVKQDASGKAWDYAKAIAKGIGAIPGGIAVISSF
EEEALLDLMSEHTWVPILFGAIKACYDIAVKEYGVSPEAALLEFYASGELAEIARLIAEEGIFNQ
MVHHSTTSQYGTLTRMFKYYDVVRRIVENEAKYIWDGSFAKEWSLEQQAGYPVFYRLWELAT
QSEMAKAEKELYKLLGRKVKNDLEVLFQGPWSHPQFEKGGGSGGGSGGGSWSHPQFE
K*

Additional SpyCatcher versions, which are available in literature as SpyCatcher 001 and 003, will also be used. Some of these new SpyCatcher variants are either full length or contain a 25 amino acids deletion on the N-terminal part of the SpyCatcher based on structure necessities. In addition, a combination of Gly-Ser linkers between the SpyCatcher and the nanoparticle are being tested.

Another strategy to improve the expression and generation of the KARI nanoparticle disclosed herein will comprise initially placing the SpyTag within the protein of interest (e.g., the antigen, to be tested) in the nanoparticle. This will be an alternative method for the coupling between the nanoparticle and the antigen.

### Example 2: Expression of the Nanoparticles

This example illustrates the expression and generation of a self-assembling KARI nanoparticles using the SpyCatcher/SpyTag ligation system.

Methods. The expression vector comprising the gene encoding the KARI nanoparticle was expressed in bacteria. The method of expressing the recombinant protein in bacterial cells is known to a person of skill in the art. For example as shown in Chen et al., J. Am. Chem. Soc. 141 (51): 19983-19987 (2019). The purification protocol of the KARI nanoparticle was substantially improved. The most important improvements were as follows.

Magnesium was no longer added as co-factor to the TBS buffer to promote the assembly of the KARI nanoparticle. This omission was important because the present inventors unexpectedly found that, magnesium precipitated the sample and the final well-formed amount of the KARI nanoparticles was significantly lower.

In addition, the method of Chen et al., J. Am. Chem. Soc. 141 (51): 19983-19987 (2019) discloses heating the bacteria at 65° C. because this heated method favored the formation of the nanoparticle. However, the present inventors found that the KARI nanoparticle did not need to be heated to 65° C. as recommended by Chen et al. and heating the bacteria before lysing the cells caused protein aggregation. In addition, using standard bacterial protein production and purification (pellet the bacteria, resuspend in TBS, mechanical breakdown to lysate the bacteria and centrifugation of the bacterial debris, followed by affinity purification) produced high and better quality 12-meric (dodecameric) KARI nanoparticle with very small fraction of aggregations and no perceptible remaining monomer.

Results. A recombinant KARI protein fused to the SpyCatcher003 was purified and a 2.6A resolution cryo_EM map of that recombinant KARI protein was obtained. These results illustrated that the recombinant KARI protein, the correct the KARI nanoparticle and the SpyCatcher were folded correctly in the produced self-assembling KARI nanoparticle. FIGS. 3A-B.

### Example 3: Expression of the Antigens

Antigens for their display on the KARI nanoparticles will be tagged in the C-terminus with the SpyTag. While most of the antigens examined can be tagged at the C-terminus, some antigens favored an N-terminus tag. Two version of the SpyTag polypeptides will be tested (SpyTag 001 and SpyTag 003) together with a Gly-Ser linker in between the antigen and the SpyTag. In some cases, the antigen will contain the SpyCatcher instead the SpyTag if the system requires it. Instead of the SpyCatcher/SpyTag, any of the substrates for the Sortase A reaction coupling can also be used. Independently, different SARS-CoV-2 VFLIP variants were flanked with the SpyCatcher (i.e., three different VOCs such D614G, Beta and Delta). SARS-CoV-2 VFLIP variants are disclosed in WO 2022/087255A2 and PCT/US2022/053783.

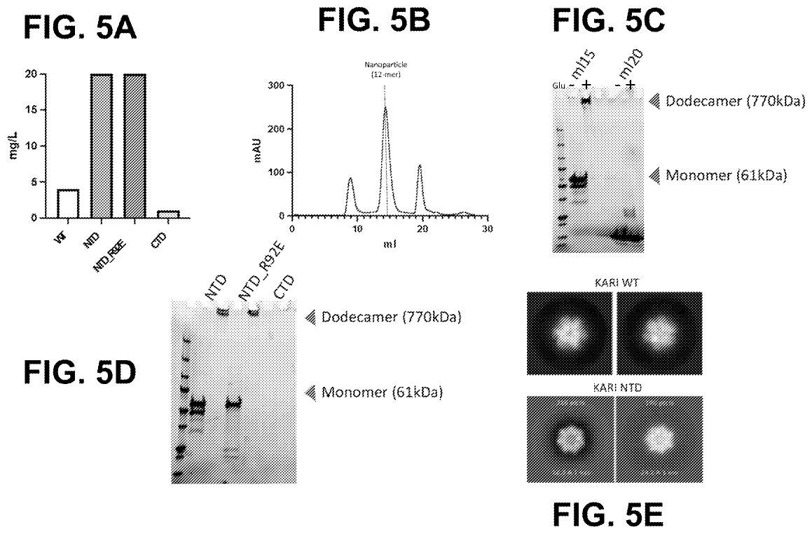

The pCMV plasmids encoding the antigen, such VFLIP or domains of the SARS-CoV-2 spike such the RBD, were transiently transfected in CHO cells under the manufacturer' specifications. One week after the transfection, the supernatant was harvested, cells were clarified and the supernatant containing the soluble VFLIP was double purified by affinity column (streptrap columns) and further purified by Size Exclusion Chromatography. FIG. 5A-E show the characterization of the recombinant KARI fused to a SARS-CoV-2 Nucleoprotein domains. FIG. 5A shows the relative expression of KARI fused to the N-Terminal Domain (NTD, RNA binding domain) and C-Terminal Domain (CTD, dimerization domain) domains of the SARS-CoV-2 Nucleoprotein and illustrates that the NTD domain purification yield was 5 times higher than that of the wild-type protein. The NTD mutant R92E indicates a nucleoprotein mutant that cannot bind RNA. FIG. 5B shows a size exclusion chromatogram of KARI on a Superdex S6i column. The pick of the 15 ml shows the self-assembled nanoparticle. FIG. 5C shows an SDS-PAGE gel of KARI of FIG. 5B with (+) or without (−) glutaraldehyde as cross-linker. The fraction that elutes in 15 ml in FIG. 5B had a molecular weight that was compatible with a self-assembled 12-meric nanoparticle with 12 SpyCatcher tags (770 kDa) after it was cross-linked. FIG. 5D shows an SDS-PAGE gel of KARI comprising the NTD (RNA binding domain) or CTD (dimerization domain) domains of the SARS-CoV-2 Nucleoprotein with (+) or without (−) the glutaraldehyde linker. FIG. 5D further illustrates that the KARI_NTD and KARI_NTD_R92E formed perfect self-assembled nanoparticles. However, neither the yield nor the size of the KARI_CTD were compatible with a well-formed nanoparticle. FIG. 5E shows representative 2D classes from negative staining electron microscopy.

### Example 4: Coupling of KARI and VFLIP

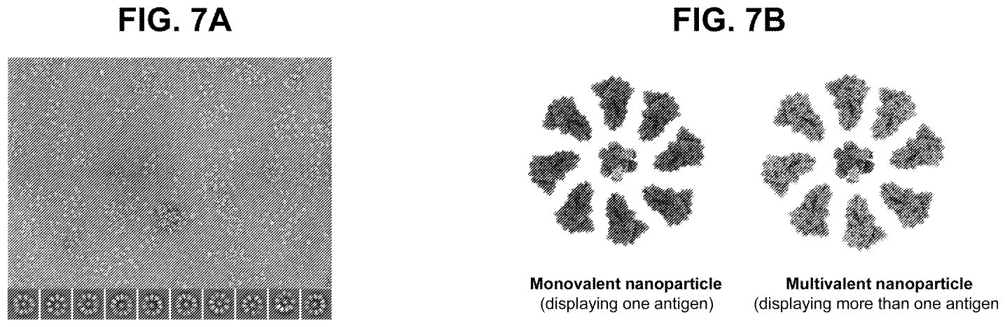

This example illustrates a method of coupling of the self-assembling KARI nanoparticles to an antigen via SpyTag/SpyCatcher. After mixing the nanoparticle and the antigen in equimolar ratio, the mixture was concentrated to about 20-50 μM and incubated overnight. The mixture was the size exclusion purified to discard the uncoupled leftovers and the resulted SARS-CoV-2 VFLIP-KARI nanoparticles bearing 12 copies of VFLIP on a monovalent nanoparticle were isolated. FIG. 7A. Alternatively, instead of coupling one antigen (i.e.; VFLIP from the Wuhan variant) to the nanoparticle, 3 VFLIPs from different variants (Wuhan, Delta and Omicron) were combined in equimolar concentration with the nanoparticle. As shown in FIG. 7B, we can obtain a multivalent nanoparticles comprising the 3 VFLIPs from different variants were obtained.

Improvement over the art. Compared to ferritin nanoparticles, the most used protein self-assembling nanoparticles used for the delivery of 8 antigens on each molecule, the 12-meric (dodecameric) nanoparticle KARI displayed 12 antigens in the surface, presumably increasing the trafficking of the antigens to the lymph nodes, and enhancing the activity of antigen presenting cells (APC). See FIGS. 3A-C and FIG. 4A-B.

The effectiveness of a prophylactic vaccine is determined by the generation of a long-lasting T-cell-dependent IgG antibody T-cells by antigen presenting cells (APC) followed by the activation of B-cells by antigen and a specialized T helper cell (the T Follicular helper cell; TFH cell) response. Generating this response involves the activation of APCs phagocytize, process, and present antigens to T-cells. When the APCs recognize, they become activated and differentiate into T helper cells. Naive B-cells require two signals to mature into high-affinity IgG plasma cells and receive a first signal from the crosslinking of multiple B-cell receptors (BCR) with an antigen that has directly entered the lymph node or was presented by APCs, followed by endocytosis of the receptor and antigen, and then presentation of the processed antigen on the surface. The second signal is delivered upon recognition of the B-cell-presented antigen with a TFH cell, which activates the B-cell to mature into an IgG-producing plasma cell.

Two characteristics of the KARI nanoparticle platforms disclosed herein contribute to generating the B-cell IgG response: (1) the attachment of the antigen to a larger scaffold, which improves APC uptake and retention in lymph follicles and (2) the repetitive array of antigens, which enables efficient binding and activation of multiple B-cell receptors. Attachment of antigens on particles increases the overall particulate size into an optimal size range for efficient uptake by APCs, which allows for greater presentation of antigen by APCs to activate T-helper cells. Larger particles are also efficiently opsonized with complement. Opsonization promotes binding to the surface of follicular dendritic cells (FDCs), elongating retention in lymph follicles, and enhancing antigen presentation to B-cells. Particles displaying numerous antigens can then facilitate B-cell activation through efficient crosslinking with multiple BCRs. High-density conjugation of an antigen to a VLP activated a specific IgG antibody response, while low-density conjugation did not (despite increased antigen quantity), suggesting an effect outside the total amount of antigen. Accordingly, the self-assembling KARI nanoparticles of the present disclosure will enhance the immunogenicity of and the affinity and maturation of the elicited B cells.

Sequence of the VFLIP_SpyTag001 tested in this example is shown below (SEQ ID NO: 10; VFLIP—Trimerization Domain—SpyTag001—Purification tags):

QCVNLTTRTQLPPAYTNSFTRGVYYPDKVFRSSVLHSTQDLFLPFFSNVTWFHAI
HVSGTNGTKRFDNPVLPFNDGVYFASTEKSNIIRGWIFGTTLDSKTQSLLIVNNATNVVI
KVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFT
VEKGIYQTSNFRVQPTESIVRFPNITNLCPFGEVENATRFASVYAWNRKRISNCVADYSV
LYNSASFSTFKCYGVSPTKLNDLCFTNVYADSFVIRGDEVRQIAPGQTGKIADYNYKLPD
DFTGCVIAWNSNNLDSKVGGNYNYLYRLFRKSNLKPFERDISTEIYQAGSTPCNGVEGF
NCYFPLQSYGFQPTNGVGYQPYRVVVLSFELLHAPATVCGPKKSTNLVKNKCVNFNFN
GLTGTGVLTESNKKFLPFQQFGRDIADTTDAVRDPQTLEILDITPCSFGGVSVITPGTNTS
NQVAVLYQGVNCTEVPVAIHADQLTPTWRVYSTGSNVFQTRAGCLIGAEHVNNSYECD
IPIGAGICASYQGGSGGSSIIAYTMSLGAENSVACSNNSIAIPTNFTISVTTEILPVSMTKTS
VDCTMYICGDSTECSNLLLQYGSFCTQLNRALTGIAVEQDKNTQEVFAQVKQIYKTPPIK
DFGGFNFSQILPDPSKPSKRSPIEDLLFNKVTLADAGFIKQYGDCLGDIAARDLICAQKFN
GLTVLPPLLTDEMIAQYTSALLAGTICSGWTFGAGPALQIPFPMQMAYRENGIGVTQNV
LYENQKLIANQFNSAIGKIQDSLSSTPSALGKLQDVVNQNAQALNTLVKQLSSNFGAISS
VLNDILSRLDKPEAEVQIDRLITGRLQSLQTYVTQQLIRAAEIRASANLAATKMSECVLG
QSKRVDFCGKGYHLMSFPQSAPHGVVFLHVTYVPAQEKNFTTAPAICHDGKAHFPREG
VFVSNGTHWFVTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQELGKYEQYIKGS
GYIPEAPRDGQAYVRKDGEWVLLSTFLAHIVMVDAYKPTKLEVLFQGPAGWSHPQFEK
GGGSGGGSGGGSWSHPQFEK*

Sequence of the VFLIP-SortaseTag tested in this example is shown below (SEQ ID NO: 14; VFLLP—Trimerization Domain—SortaseA—Purification tars):

QCVNLTTRTQLPPAYTNSFTRGVYYPDKVFRSSVLHSTQDLFLPFFSNVTWFHAI
HVSGTNGTKRFDNPVLPFNDGVYFASTEKSNIIRGWIFGTTLDSKTQSLLIVNNATNVVI
KVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFT
VEKGIYQTSNFRVQPTESIVRFPNITNLCPFGEVFNATRFASVYAWNRKRISNCVADYSV
LYNSASFSTFKCYGVSPTKLNDLCFTNVYADSFVIRGDEVRQIAPGQTGKIADYNYKLPD
DFTGCVIAWNSNNLDSKVGGNYNYLYRLFRKSNLKPFERDISTEIYQAGSTPCNGVEGF
NCYFPLQSYGFQPTNGVGYQPYRVVVLSFELLHAPATVCGPKKSTNLVKNKCVNFNFN
GLTGTGVLTESNKKFLPFQQFGRDIADTTDAVRDPQTLEILDITPCSFGGVSVITPGTNTS
NQVAVLYQGVNCTEVPVAIHADQLTPTWRVYSTGSNVFQTRAGCLIGAEHVNNSYECD
IPIGAGICASYQGGSGGSSIIAYTMSLGAENSVACSNNSIAIPTNFTISVTTEILPVSMTKTS
VDCTMYICGDSTECSNLLLQYGSFCTQLNRALTGIAVEQDKNTQEVFAQVKQIYKTPPIK
DFGGFNFSQILPDPSKPSKRSPIEDLLFNKVTLADAGFIKQYGDCLGDIAARDLICAQKFN
GLTVLPPLLTDEMIAQYTSALLAGTICSGWTFGAGPALQIPFPMQMAYRFNGIGVTQNV
LYENQKLIANQFNSAIGKIQDSLSSTPSALGKLQDVVNQNAQALNTLVKQLSSNFGAISS
VLNDILSRLDKPEAEVQIDRLITGRLQSLQTYVTQQLIRAAEIRASANLAATKMSECVLG
QSKRVDFCGKGYHLMSFPQSAPHGVVFLHVTYVPAQEKNFTTAPAICHDGKAHFPREG
VFVSNGTHWFVTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQELGKYEQYIKGS
GYIPEAPRDGQAYVRKDGEWVLLSTFLGGSGGSLPETGGGSGGSLEVLFQGPAGWSHP
QFEKGGGSGGGSGGGSWSHPQFEK*

Additional SARS-CoV-2 VFLIP variants are disclosed in See e.g., PCT/US2021/056037 (WO 2022/087255A2) and PCT/US2022/053783, which are incorporated herein by reference in their entirety.

For example, an additional SARS-CoV-2 VFLIP variant can comprise the amino acid sequence of SEQ ID NO: 59, SEQ ID NO: 60, SEQ ID NO: 61, or SEQ ID NO: 62.

The SARS-CoV-2 VFLIP variant can be a VFLIP_Omicron (Omicron (e.g., B.1.1.529)). Mutations and the amino acid sequence of VFLIP_Omicron (Omicron (B.1.1.529)) is shown in SEQ ID NO: 59 below. Mutations are in Bold, Linker in bold underlined. additional mutations in italics.

(SEQ ID NO: 59)
QCVNLTTRTQLPPAYTNSFTRGVYYPDKVFRSSVLHSTQDLFLPFFSNVTWFHVI
SGTNGTKRFDNPVLPFNDGVYFASIEKSNIIRGWIFGTTLDSKTQSLLIVNNATNVVIKVC
EFQFCNDPFLDHKNNKSWMESEFRVYSSANNCTFEYVSQPFLMDLEGKQGNFKNLREF
VFKNIDGYFKIYSKHTPILVRDLPQGFSALEPLVDLPIGINITRFQTLLALHRSYLTPGDSSS
GWTAGAAAYYVGYLQPRTFLLKYNENGTITDAVDCALDPLSETKCTLKSFTVEKGIYQT
SNFRVQPTESIVRFPNITNLCPFDEVFNATRFASVYAWNRKRISNCVADYSVLYNLAPFF
TFKCYGVSPTKLNDLCFTNVYADSFVIRGDEVRQIAPGQTGNIADYNYKLPDDFTGCVI
AWNSNKLDSKVSGNYNYLYRLFRKSNLKPFERDISTEIYQAGNKPCNGVAGENCYFPL
RSYSFRPTYGVGHQPYRVVVLSFELLHAPATVCGPKKSTNLVKNKCVNFNFNGLKGTG
VLTESNKKFLPFQQFGRDIADTTDAVRDPQTLEILDITPCSFGGVSVITPGTNTSNQVAVL
YQGVNCTEVPVAIHADQLTPTWRVYSTGSNVFQTRAGCLIGAEYVNNSYECDIPIGAGI
CASYQGGSGGSSIIAYTMSLGAENSVACSNNSIAIPTNFTISVTTEILPVSMTKTSVDCTM
YICGDSTECSNLLLQYGSFCTQLKRALTGIAVEQDKNTQEVFAQVKQIYKTPPIKYFGGF
NFSQILPDPSKPSKRSPIEDLLFNKVTLADAGFIKQYGDCLGDIAARDLICAQKFKGLTVL
PPLLTDEMIAQYTSALLAGTICSGWTFGAGPALQIPFPMQMAYRENGIGVTQNVLYENQ
KLIANQFNSAIGKIQDSLSSTPSALGKLQDVVNHNAQALNTLVKQLSSKFGAISSVLNDI
FSRLDKPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSN
GTHWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQ.

The SARS-CoV-2 VFLIP variant can be a VFLIP_Omicron_BA.2. The amino acid sequence of the VFLIP_Omicron_BA.2 is shown in SEQ ID NO: 60 below. Mutations are shown in BOLD.

(SEQ ID NO: 60)
QCVNLITRTQSYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIKVC
EFQFCNDPFLDVYYHKNNKSWMESEFRVYSSANNCTFEYVSQPFLMDLEGKQGNFKNL
REFVFKNIDGYFKIYSKHTPINLGRDLPQGFSALEPLVDLPIGINITRFQTLLALHRSYLTP
GDSSSGWTAGAAAYYVGYLQPRTFLLKYNENGTITDAVDCALDPLSETKCTLKSFTVEK
GIYQTSNFRVQPTESIVRFPNITNLCPFDEVFNATRFASVYAWNRKRISNCVADYSVLYN
FAPFFAFKCYGVSPTKLNDLCFTNVYADSFVIRGNEVSQIAPGQTGNIADYNYKLPDDFT
GCVIAWNSNKLDSKVGGNYNYLYRLFRKSNLKPFERDISTEIYQAGNKPCNGVAGFNC
YFPLRSYGFRPTNGVGHQPYRVVVLSFELLHAPATVCGPKKSTNLVKNKCVNFNFNGL
TGTGVLTESNKKFLPFQQFGRDIADTTDAVRDPQTLEILDITPCSFGGVSVITPGTNTSNQ
VAVLYQGVNCTEVPVAIHADQLTPTWRVYSTGSNVFQTRAGCLIGAEYVNNSYECDIPI
GAGICASYQGGSGGSSIIAYTMSLGAENSVACSNNSIAIPTNFTISVTTEILPVSMTKTSV
DCTMYICGDSTECSNLLLQYGSFCTQLKRALTGIAVEQDKNTQEVFAQVKQIYKTPPIKY
FGGFNFSQILPDPSKPSKRSPIEDLLFNKVTLADAGFIKQYGDCLGDIAARDLICAQKENG
LTVLPPLLTDEMIAQYTSALLAGTICSGWTFGAGPALQIPFPMQMAYRFNGIGVTQNVL
YENQKLIANQFNSAIGKIQDSLSSTPSALGKLQDVVNHNAQALNTLVKQLSSKFGAISSV
LNDILSRLDKPEAEVQIDRLITGRLQSLQTYVTQQLIRAAEIRASANLAATKMSECVLGQ
SKRVDFCGKGYHLMSFPQSAPHGVVFLHVTYVPAQEKNFTTAPAICHDGKAHFPREGV
FVSNGTHWFVTQRNFYEPQIITTDNTFVSGNCDVVIGIVNNTVYDPLQPELDSFKEELDK
YFKNHTSPDVDLGDISGINASVVNIQKEIDRLNEVAKNLNESLIDLQELGKYEQ.

The SARS-CoV-2 VFLIP variant can be a VFLIP_BA4.Beta. The amino acid sequence of the VFLIP_BA4.Beta is shown in SEQ ID NO: 6.

(SEQ ID NO: 61)
QCVNLITRTQSYTNSFTRGVYYPDKVFRSSVLHSTQDLFLPFFSNVTWFHAISGTN
GTKRFDNPVLPFNDGVYFASTEKSNIIRGWIFGTTLDSKTQSLLIVNNATNVVIKVCEFQF
CNDPFLDVYYHKNNKSWMESEFRVYSSANNCTFEYVSQPFLMDLEGKQGNFKNLREFV
FKNIDGYFKIYSKHTPINLGREPEDLPQGFSALEPLVDLPIGINITRFQTLLALHRSYLTPG
DSSSGWTAGAAAYYVGYLQPRTFLLKYNENGTITDAVDCALDPLSETKCTLKSFTVEKG
IYQTSNFRVQPTESIVRFPNITNLCPFDEVFNATRFASVYAWNRKRISNCVADYSVLYNF
APFFAFKCYGVSPTKLNDLCFTNVYADSFVIRGNEVSQIAPGQTGNIADYNYKLPDDFTG
CVIAWNSNKLDSKVGGNYNYRYRLFRKSNLKPFERDISTEIYQAGNKPCNGVAGVNCY
FPLQSYGFRPTYGVGHQPYRVVVLSFELLHAPATVCGPKKSTNLVKNKCVNFNFNGLT
GTGVLTESNKKFLPFQQFGRDIADTTDAVRDPQTLEILDITPCSFGGVSVITPGTNTSNQV
AVLYQGVNCTEVPVAIHADQLTPTWRVYSTGSNVFQTRAGCLIGAEYVNNSYECDIPIG
AGICASYQGGSGGSSIIAYTMSLGAENSVACSNNSIAIPTNFTISVTTEILPVSMTKTSVDC
TMYICGDSTECSNLLLQYGSFCTQLKRALTGIAVEQDKNTQEVFAQVKQIYKTPPIKYFG
GFNFSQILPDPSKPSKRSPIEDLLFNKVTLADAGFIKQYGDCLGDIAARDLICAQNFKGLT
VLPPLLTDEMIAQYTSALLAGTICSGWTFGAGPALQIPFPMQMAYRENGIGVTQNVLYE
NQKLIANQFNSAIGKIQDSLSSTPSALGKLQDVVNHNAQALNTLVKQLSSKFGAISSVLN
DILSRLDKPEAEVQIDRLITGRLQSLQTYVTQQLIRAAEIRASANLAATKMSECVLGQSK
RVDFCGKGYHLMSFPQSAPHGVVFLHVTYVPAQEKNFTTAPAICHDGKAHFPREGVFV
SNGTHWFVTQRNFYEPQIITTDNTFVSGNCDVVIGIVNNTVYDPLQPELDSFKEELDKYF
KNHTSPDVDLGDISGINASVVNIQKEIDRLNEVAKNLNESLIDLQELGKYEQ.

The SARS-CoV-2 VFLIP variant can be a VFLIP-BQ.1.1. The amino acid sequence of VFLIP_BQ.1.1 is shown in SEQ ID NO: 62.

(SEQ ID NO: 62)
QCVNLITRTQSYTNSFTRGVYYPDKVFRSSVLHSTQDLFLPFFSNVTWFHAISGTN
GTKRFDNPVLPFNDGVYFASTEKSNIIRGWIFGTTLDSKTQSLLIVNNATNVVIKVCEFQF
CNDPFLDVYYHKNNKSWMESEFRVYSSANNCTFEYVSQPFLMDLEGKQGNFKNLREFV
FKNIDGYFKIYSKHTPINLGREPEDLPQGFSALEPLVDLPIGINITRFQTLLALHRSYLTPG
DSSSGWTAGAAAYYVGYLQPRTFLLKYNENGTITDAVDCALDPLSETKCTLKSFTVEKG
IYQTSNFRVQPTESIVRFPNITNLCPFDEVFNATTFASVYAWNRKRISNCVADYSVLYNF
APFFAFKCYGVSPTKLNDLCFTNVYADSFVIRGNEVSQIAPGQTGNIADYNYKLPDDFTG
CVIAWNSNKLDSTVGGNYNYRYRLFRKSKLKPFERDISTEIYQAGNKPCNGVAGVNCY
FPLQSYGFRPTYGVGHQPYRVVVLSFELLHAPATVCGPKKSTNLVKNKCVNFNFNGLT
GTGVLTESNKKFLPFQQFGRDIADTTDAVRDPQTLEILDITPCSFGGVSVITPGTNTSNQV
AVLYQGVNCTEVPVAIHADQLTPTWRVYSTGSNVFQTRAGCLIGAEYVNNSYECDIPIG
AGICASYQGGSGGSSIIAYTMSLGAENSVACSNNSIAIPTNFTISVTTEILPVSMTKTSVDC
TMYICGDSTECSNLLLQYGSFCTQLKRALTGIAVEQDKNTQEVFAQVKQIYKTPPIKYFG
GFNFSQILPDPSKPSKRSPIEDLLFNKVTLADAGFIKQYGDCLGDIAARDLICAQNFKGLT
VLPPLLTDEMIAQYTSALLAGTICSGWTFGAGPALQIPFPMQMAYRENGIGVTQNVLYE
NQKLIANQFNSAIGKIQDSLSSTPSALGKLQDVVNHNAQALNTLVKQLSSKFGAISSVLN
DILSRLDKPEAEVQIDRLITGRLQSLQTYVTQQLIRAAEIRASANLAATKMSECVLGQSK
RVDFCGKGYHLMSFPQSAPHGVVFLHVTYVPAQEKNFTTAPAICHDGKAHFPREGVFV
SNGTHWFVTQRNFYEPQIITTDNTFVSGNCDVVIGIVNNTVYDPLQPELDSFKEELDKYF
KNHTSPDVDLGDISGINASVVNIQKEIDRLNEVAKNLNESLIDLQELGKYEQ.

### Example 5: Universal Vaccine Platform

This example provides methods of generating universal vaccine comprising the self-assembling KARI nanoparticle.

The next generation of pan-coronavirus vaccines can comprise KARI nanoparticles containing conserved epitopes from the SARS-CoV-2 spike and the nucleocapsid to present B and T epitopes.

The ‘Intra-viral’ Nucleocapsid (N) protein can be used as a novel vaccine strategy together with the spike S protein. The N-protein sequence is relatively conserved within any given strain of virus during spread and evolution and across different strains of coronaviruses. A successful N-protein vaccine using the self-assembling KARI nanoparticle can be useful against several viruses within the coronavirus family and can effectively trigger the cellular T cell response.

For example, although most neutralizing antibodies target the SARS-CoV-2 spike S1 subunit, the S2 subunit is most conserved. The conserved structure of the S2 subunit can likely be constrained by the complex and precisely timed refolding events essential for productive viral entry. Nevertheless, antibodies targeting this region may be neutralizing and protective against infection and pathology in vivo. Also, it has been reported that SARS-CoV-2-infected children and young adolescents recently infected with an endemic HCoV (seasonal circulating common cold coronavirus such as Oc43, HKU1, 229E) develop higher titers of S2 antibodies than SARS-CoV-2-infected adults without a recognized antecedent endemic HCoV infection. In individuals not previously infected with SARS-CoV-2, a majority of the anti-SARS-CoV-2 immunoglobulin G (IgG) repertoire targets the S2 subunit, underscoring the potential for cross-reactivity directed at conserved epitopes on HCoVs. Accordingly, the S2 subunit serves as an attractive target for the development of broad-spectrum vaccines and therapeutic antibodies for current and future novel HCoVs.

As such, KARI nanoparticles containing the S2 subunit of SARS-CoV-2 can be generated and the S2 subunit of SARS-CoV-2 can be stabilized by the VFLIP technology. A self-assembling KARI nanoparticle displaying the S2 subunit SARS-CoV-2 VFLIP can elicit a robust pan-CoV protective antibody response and together with different potent T-cell epitopes derived from conserved and very immunological epitopes front the SARS-CoV-2 N nucleoprotein (i.e., N epitope 104-116). FIGS. 4A-B. Furthermore, the N epitopes can be genetically fused to the KARI nanoparticle in the C-term and the spike S2 subunit can be synthesized as mentioned before for the VFLIP spike protein.

This example shows that the present inventors have been able to successfully display domains of the SARS-CoV-2 N (Nucleoprotein) on KARI nanoparticles. FIGS. 5A-E show that, when KARI nanoparticle is expressed together in the 3′ of the gene, right after the nanoparticle, with domains of the SARS-CoV N (especially the NTD domain) the resulting KARI nanoparticle expresses 5 times better that the KARI nanoparticle along. After an SEC purification, in SDS-PAGE, a monomeric and the 12-meric bands, without or with glutaraldehyde as crosslinker was detected. In addition, a 2D reconstruction of the KARI nanoparticles in negative staining electron microscopy (NS_EM) was obtained.

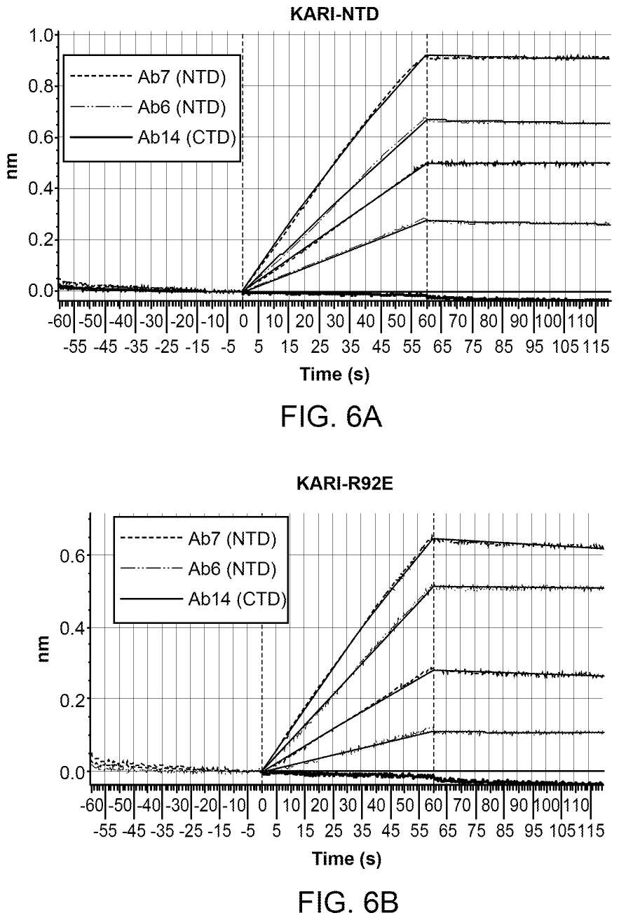

Finally, to determine whether or not the N-Terminal domain (NTD) from the SARS-CoV-2 N was successfully expressed and folded, binding experiments Octed) with two anti-N_NTD antibodies isolated in the laboratory was performed. These experiments showed that there were a high affinity binding of those two antibodies for the KARI_NTD nanoparticle. These results confirmed the presence of 12 well-formed NTD_N domains in the C-terminal part of the nanoparticle. FIGS. 6A-B.

### Example 6: Multivalent System to Deliver Short T-Cell Epitopes

This example illustrates how the KARI nanoparticles are used as a multivalent system to deliver T-cell epitopes.

The KARI nanoparticles disclosed herein are used to display different molecules of interest (e.g., antigens, antibodies, drugs, adjuvants) via a coupling system (e.g., SpyTag/SpyCatcher system, Sortase A system, etc.) to generate a multivalent system capable of delivering T-cell epitopes.

In one example, the KARI nanoparticles are used to display the SARS-CoV-2 RBDs domains from different variants of concern. The immunogenicity of these KARI-SARS-CoV-2 RBD nanoparticles are evaluated of in mice or hamsters. Ultimately, these KARI-SARS-CoV-2 RBD nanoparticles are used to generate a broadly SARS-CoV-2 universal vaccine candidate.

Second, the KARI nanoparticles are used to display different SARS-CoV-2 spikes stabilized in the pre-fusion conformation with the VFLIP technology, to measure whether or not the immunization in a multimeric environment (nanoparticle) may induce a higher and more durable neutralizing antibody response than the soluble spike subunits alone.

Lastly, the KARI nanoparticles are used as a multivalent system to deliver short T-cell epitopes. It has been demonstrated that short peptides elicit poor immune responses and the best T-cell epitopes that may be included on a future SARS-CoV-2 vaccine are difficult to map. Accordingly, genetically modified T-cell epitopes are fused to the KARI nanoparticle to significantly increase the immunogenicity to those epitopes. The uses of the nanoparticle as disclosed herein is not limited to SARS-CoV-2, as any antigens, including but not limited to bacterial, viral, or tumor antigens, can be used.

### Embodiments

In one embodiment, provided herein is a self-assembling nanoparticle comprising an amino acid sequence of a recombinant Ketol-acid reductoisomerase (KARI; EC 1.1.1.86) and a tether sequence operably linking the KARI to at least one protein or peptide of interest wherein each protein comprises a capture sequence.

In a separate embodiment, the self-assembling nanoparticle of embodiment 1, further comprises a purification tag, optionally wherein the purification tag is operably linked to a C-terminus or an N-terminus of the recombinant KARI.

In a separate embodiment, he self-assembling nanoparticle of embodiment 1 or 2 further comprises a T-cell epitope, operably linked to the C-terminus or the N-terminus of the recombinant KARI.

In a separate embodiment, in the self-assembling nanoparticle of any one of embodiments 1-3, the recombinant KARI is an archeal, bacterial, or proteobacteria KARI.

In a separate embodiment, in the self-assembling nanoparticle of any one of embodiments 1-4, the recombinant KARI is a dodecameric KARI.

In a separate embodiment, in the self-assembling nanoparticle of any one of embodiments 1-5, the recombinant KARI is selected from Methanothermococcus thermolithotrophicus (MtKARI), Helicobacter pylori, Pseudomonas Aeruginosa (PaKARI), Saccharolobus solfataricus (SacsKARI), Sulfolobus solfataricus (Sso-KARI), A. vinelandii, Sulfolobus sp. E5-1-F, Sulfolobus islandicus, Saccharolobus shibatae, Saccharolobus caldissimus, Saccharolobus shibatae, Stygiolobus sp. KN-1, Sulfolobus sp., Sulfodiicoccus acidiphilus, Acidianus brierleyi, or Acidianus manzaensis, Thaumarchaeota archeon, Candidatus Bathyarchaeota, Nitrososphaeria archaeon, Dictyoglomi bacterium, Vulcanisaeta sp, Deltaproteobacteria bacterium, or Acidiplasma cupricumulans

In a separate embodiment, in the self-assembling nanoparticle of any one of embodiments 1-6, wherein the recombinant KARI is a dodecameric KARI selected from M. thermolithotrophicus (MtKARI), H. pylori, P. Aeruginosa (PaKARI), S. solfataricus (SacsKARI), or S. solfataricus (Sso-KARI).

In a separate embodiment, in the self-assembling nanoparticle of any one of embodiments 5-8, the dodecameric KARI nanoparticle displays at least about twelve molecules of the protein of interest on the surface of the dodecameric KARI nanoparticle.

In a separate embodiment, the self-assembling nanoparticle of any one of embodiments 5-8, further comprises a detectable label.

In a separate embodiment, the self-assembling nanoparticle of any one of embodiments 5-8, further comprises a purification tag.

In a separate embodiment, in the self-assembling nanoparticle of any one of embodiments 1-10, wherein the recombinant KARI comprises the amino acid sequence of SEQ ID NO: 1 or 2, or an amino acid sequence having at least about 37%, at least about 40%, at least about 50%, at least about 55%, at least about 65%, at least about 70%, at least about 75%, at least about 80%, at least about 85%, at least about 90%, at least about 95%, or at least about 99% identity to the amino acid sequence of SEQ ID NO: 1 or 2.

In a separate embodiment, in the self-assembling nanoparticle of any one of embodiments 1-11, the recombinant KARI comprises the amino acid sequence of SEQ ID NO: 2.

In a separate embodiment, in the self-assembling nanoparticle of any one of embodiments 1-12, the recombinant KARI comprises a deletion in the amino acid sequence of SEQ ID NO: 1.

In a separate embodiment, in the self-assembling nanoparticle of any one of embodiments 1-13, the recombinant KARI comprises a deletion of about 2, about 3, about 5, about 10, about 12, about 15, or about 20 amino acids in the N-terminus of SEQ ID NO: 1.

In a separate embodiment, in the self-assembling nanoparticle of any one of embodiments 1-13, the recombinant KARI comprises a deletion of at least 8 amino acids in the N-terminus of SEQ ID NO: 1.

In a separate embodiment, in the self-assembling nanoparticle of any one of embodiments 1-15, the tether sequence:
(a) is selected from SpyTag, SpyTag001, SpyTag002, SpyTag003, SpyCatcher, SpyCatcher001, SpyCatcher002, SpyCatcher003, SnoopTag, SnoopCatcher, a sortase recognition domain, a sortase bridging domain, a butelase recognition motif, a C-peptide, a split intein, or a peptiligase substrate; or
(b) comprises the amino acid sequence of SEQ ID NO: 4, 8, 13, or 15 or an amino acid sequence having at least about 70%, at least about 75%, at least about 80%, at least about 85%, at least about 90%, at least about 95%, at least about 97%, or at least about 99% identity to the amino acid sequence of SEQ ID NO: 4, 8, 13, or 15 across the full length of the amino acid sequence, respectively.

In a separate embodiment, in the self-assembling nanoparticle of any one of embodiments 1-16, the capture sequence is selected from SpyTag, SpyTag001, SpyTag002, SpyTag003, SpyCatcher, SpyCatcher001, SpyCatcher002, SpyCatcher003, SnoopTag, SnoopCatcher, a sortase recognition domain, a sortase bridging domain, a butelase recognition motif, a C-peptide, a split intein, or a peptiligase substrate.

In a separate embodiment, in the self-assembling nanoparticle of any one of embodiments 1-17:
(a) the tether sequence is a SpyCatcher and the capture sequence is SpyTag;
(b) the tether sequence comprises the amino acid sequence of SEQ ID NO: 4 and the capture sequence comprises the amino acid sequence of SEQ ID NO: 13;
(c) the tether sequence is a SpyTag and the capture sequence is SpyCatcher;
(d) the tether sequence comprises the amino acid sequence of SEQ ID NO: 13 and the capture sequence comprises the amino acid sequence of SEQ ID NO: 4;
(e) the tether sequence is a sortase Tag and the capture sequence is sortase A;
(f) the tether sequence is a sortase A and the capture sequence is sortase Tag;
(g) the tether sequence comprises the amino acid sequence of SEQ ID NO: 8 and the capture sequence comprises the amino acid sequence of SEQ ID NO: 15;
(h) the tether sequence comprises the amino acid sequence of SEQ ID NO: 15 and the capture sequence comprises the amino acid sequence of SEQ ID NO: 8.

In a separate embodiment, in the self-assembling nanoparticle of any one of embodiments 1-18, the self-assembling nanoparticle further comprises a linker, optionally wherein the linker operably links the recombinant KARI to the tether sequence.

In a separate embodiment, in the self-assembling nanoparticle of embodiment 19, the linker is a glycine-serine linker, or the linker comprises the amino acid sequence of SEQ ID NO: 5 or 9 or an amino acid sequence having at least about 70%, at least about 75%, at least about 80%, at least about 85%, at least about 90%, at least about 95%, at least about 97%, or at least about 99% identity to the amino acid sequence of SEQ ID NO: 5 or 9 across the full length of the amino acid sequence, respectively.

In a separate embodiment, in the self-assembling nanoparticle of any one of embodiments 2-20, the purification tag is selected from hexahistidine tag (SEQ ID NO: 65) (his-tag), Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), or fungal avidin-like protein (Tamavidin).

In a separate embodiment, in the self-assembling nanoparticle of any one of embodiments 2-21, the purification tag comprises the amino acid sequence of SEQ ID NO: 6 or an amino acid sequence having at least about 70%, at least about 75%, at least about 80%, at least about 85%, at least about 90%, at least about 95%, at least about 97%, or at least about 99% identity to the amino acid sequence of SEQ ID NO: 6 across the full length of the amino acid sequence.

In a separate embodiment, in the self-assembling nanoparticle of any one of embodiments 1-22, the protein of interest is selected from an antigen, a tumor antigen, a viral antigen, a fungal antigen, a bacterial antigen, an antigen for the development of a vaccine, an antibody, an antibody fragment, a single chain antibody, scFv, scFab, single domain antibody, a protein scaffolds, an enzyme, a hormone, or an interleukin.

In a separate embodiment, in the self-assembling nanoparticle of any one of embodiments 1-23, the protein of interest is a viral antigen selected from:
(a) a coronavirus;
(b) SARS-CoV-1, MERS-CoV, 229E (alpha), NL63 (alpha), OC43 (beta), HKU1 (beta), or SARS-CoV-2; or
(c) a SARS-CoV-2 variant selected from B.1.1.7, B.1.1.7 with E484K, B.1.135, B.1.351, P.1, B.1.427, D614G, B.1.1351, B.1.429, Lambda (C.37), Mu (B.1.621).

In a separate embodiment, the self-assembling nanoparticle of any one of embodiments 1-24, the protein of interest is:
(a) a coronavirus spike protein;
(b) a mutant coronavirus spike protein;
(c) a mutant coronavirus spike protein comprising 1, 2, 3, 4, or 5 proline mutations selected from F817P, A892P, A899P, A942P, P986K, K986P, V987P, and P987V; or
(d) a mutant coronavirus spike protein comprising at least one additional disulfide bond selected from F43C-G566C, G413C-P987C, Y707C-T883C, G1035C-V1040C, A701C-Q787C, G667C-L864C, V382C-R983C, and I712C-I816C.

In a separate embodiment, in the self-assembling nanoparticle of embodiment 25, the mutant coronavirus spike protein comprises the amino acid sequence of SEQ ID NO: 11 or an amino acid sequence having at least about 70%, at least about 75%, at least about 80%, at least about 85%, at least about 90%, at least about 95%, at least about 97%, or at least about 99% identity to the amino acid sequence of SEQ ID NO:11 across the full length of the amino acid sequence.

In a separate embodiment, in the self-assembling nanoparticle of any one of embodiment 1-26, the protein of interest is operably linked to a purification tag, and/or a trimerization motif.

In a separate embodiment, in the self-assembling nanoparticle of embodiment 27, the trimerization motif is a T4 fibritin foldon, an HIV-I -derived molecular clamp, or a viral capsid protein SHP.

In a separate embodiment, in the self-assembling nanoparticle of embodiment 27 or 28, the trimerization motif comprises the amino acid sequence of SEQ ID NO: 12, 21, or 22 or an amino acid sequence having at least about 70%, at least about 75%, at least about 80%, at least about 85%, at least about 90%, at least about 95%, at least about 97%, or at least about 99% identity to the amino acid sequence of SEQ ID NO: 12, 21, or 22, across the full length of the amino acid sequence, respectively.

In a separate embodiment, in the self-assembling nanoparticle of any one of embodiments 3-29, the T-cell epitope:
(a) is selected from a universal DR epitope (PADRE) T-helper epitope, a CpG-oligodeoxynucleotides (CpG-ODNs), a multi-epitope long peptide, a SARS-CoV-2 nucleo capsid protein N-terminal domain, a SARS-CoV-2 nucleoprotein T-cell epitope;
(b) comprises the amino acid sequence of SEQ ID NO: 23-27 or an amino acid sequence having at least about 70%, at least about 75%, at least about 80%, at least about 85%, at least about 90%, at least about 95%, at least about 97%, or at least about 99% identity to the amino acid sequence of SEQ ID NO: 23-27 across the full length of the amino acid sequence, respectively; or
(c) a SARS-CoV-2 nucleoprotein epitope comprising the amino acid sequence of SEQ ID NO: 26 or 27.

In a separate embodiment, provided is a combination of vectors comprising the self-assembling nanoparticle of any one of embodiments 1-30, wherein the combination of vectors comprises:
(a) a first vector comprising a nucleic acid sequence encoding the recombinant KARI, and a nucleic acid sequence encoding the tether sequence; or
(b) a first vector comprising a nucleic acid sequence encoding the recombinant KARI, a nucleic acid sequence encoding the tether sequence, and a nucleic acid encoding the purification Tag; and
(c) a second vector comprising a nucleic acid sequence encoding a protein of interest and a nucleic acid sequence encoding the capture sequence.

In a separate embodiment, provided is a combination of vectors comprising the self-assembling nanoparticle of any one of embodiments 1-30, the combination of vectors comprises:
(a) a first vector comprising a nucleic acid sequence encoding the recombinant KARI, a nucleic acid sequence encoding the tether sequence, and a nucleic acid encoding the T-cell epitope; or
(b) a first vector comprising a nucleic acid sequence encoding the recombinant KARI, a nucleic acid sequence encoding the tether sequence, a nucleic acid encoding the T-cell epitope, and a nucleic acid encoding the purification Tag; and
(c) a second vector comprising a nucleic acid sequence encoding a protein of interest and a nucleic acid sequence encoding the capture sequence.

In a separate embodiment, in the vector of embodiment 31 or 32, the first vector is expressed in a bacterial cell and the second vector is expressed in a mammalian cell line.

In a separate embodiment, in the vector of embodiment 31 or 32, the first vector encodes a polypeptide having the amino acid sequence of SEQ ID NO: 3 or 7 and the second vector encodes a polypeptide having the amino acid sequence of SEQ ID NO: 10 or 14.

In a separate embodiment, in the vector of embodiment of any one of embodiments 32-34, when the first and second vectors are brought together, the tether sequence and the capture sequence form a covalent bond with one another either spontaneously or with the help of an enzyme to generate an immunogenic polypeptide comprising the self-assembling nanoparticle fused to the protein of interest.

In a separate embodiment, provided is an immunogenic conjugate comprising:
(a) a self-assembling nanoparticle comprising the amino acid sequence of a recombinant Ketol-acid reductoisomerase (KARI; EC 1.1.1.86), and a tether sequence;
(b) a protein of interest comprising a capture sequence; wherein the capture sequence and the tether sequence form a covalent bond that operably links the self-assembling nanoparticle to the protein of interest.

In a separate embodiment, provided is an immunogenic conjugate of embodiment 36, further comprising a purification tag, optionally wherein the purification tag is operably linked to a C-terminus or an N-terminus of the recombinant KARI.

In a separate embodiment, provided is an immunogenic conjugate of embodiment 36 or 37 further comprising a T-cell epitope, optionally wherein the T-cell epitope is operably linked to the C-terminus or the N-terminus of the recombinant KARI.

In a separate embodiment, provided is an immunogenic conjugate of any one of embodiments 36-38, the recombinant KARI is a dodecameric KARI.

In a separate embodiment, provided is an immunogenic conjugate of any one of embodiments 36-39, wherein the recombinant KARI:
(a) is an archeal, bacterial, or proteobacteria KARI;
(b) is selected from Methanothermococcus thermolithotrophicus (MtKARI), Helicobacter pylori, Pseudomonas Aeruginosa (PaKARI), Saccharolobus solfataricus (SacsKARI), Sulfolobus solfataricus (Sso-KARI), or A. vinelandii, Sulfolobus sp. E5-1-F, Sulfolobus islandicus, Saccharolobus shibatae, Saccharolobus caldissimus, Saccharolobus shibatae, Stygiolobus sp. KN-1, Sulfolobus sp., Sulfodiicoccus acidiphilus, Acidianus brierleyi, or Acidianus manzaensis; or
(c) is a dodecameric KARI selected from M. thermolithotrophicus (MtKARI), Helicobacter pylori, Pseudomonas, Aeruginosa (PaKARI), Saccharolobus solfataricus (SacsKARI), Sulfolobus solfataricus (Sso-KARI), or A. vinelandii.

In a separate embodiment, provided is an immunogenic conjugate of any one of embodiments 36-40, wherein the recombinant KARI oligomerizes into a dodecameric (12-mer) nanoparticle.

In a separate embodiment, provided is an immunogenic conjugate of any one of embodiments 36-41, wherein the recombinant KARI oligomerizes into a dodecameric KARI nanoparticle and displays the protein of interest on the surface of the nanoparticle.

In a separate embodiment, provided is an immunogenic conjugate of embodiment 42, wherein the dodecameric KARI nanoparticle displays at least about twelve molecules of the protein of interest on the surface of the dodecameric KARI nanoparticle.

In a separate embodiment, provided is an immunogenic conjugate of any one of embodiments 36-43, wherein the recombinant KARI comprises the amino acid sequence of SEQ ID NO: 1 or 2, or an amino acid sequence having at least about 37%, at least about 40%, at least about 50%, at least about 55%, at least about 65%, at least about 70%, at least about 75%, at least about 80%, at least about 85%, at least about 90%, at least about 95%, or at least about 99% identity to the amino acid sequence of SEQ ID NO: 1 or 2 across the full length of the amino acid sequence, respectively.

In a separate embodiment, provided is an immunogenic conjugate of any one of embodiments 36-44, wherein the recombinant KARI comprises the amino acid sequence of SEQ ID NO: 2.

In a further In a separate embodiment, in the immunogenic conjugate of any one of embodiments 36-45, the recombinant KARI comprises a deletion in the amino acid sequence of SEQ ID NO: 1.

In a separate embodiment, in the immunogenic conjugate of any one of embodiments 36-46, wherein the recombinant KARI comprises a deletion of about 2, about 3, about 5, about 10, about 12, about 15, or about 20 amino acids in the N-terminus of SEQ ID NO: 1.

In a separate embodiment, in the immunogenic conjugate of embodiment 46 or 47, the deletion enhances the expression and purification of the self-assembly nanoparticle when compared to the expression and purification of a wild-type KARI nanoparticle.

In a separate embodiment, in the immunogenic conjugate of any one of embodiments 36-48, the tether sequence:
(a) is selected from SpyTag, SpyTag001, SpyTag002, SpyTag003, SpyCatcher, SpyCatcher001, SpyCatcher002, SpyCatcher003, SnoopTag, SnoopCatcher, a sortase recognition domain, a sortase bridging domain, a butelase recognition motif, a C-peptide, a split intein, or a peptiligase substrate; or
(b) comprises the amino acid sequence of SEQ ID NO: 4, 8, 13, or 15 or an amino acid sequence having at least about 70%, at least about 75%, at least about 80%, at least about 85%, at least about 90%, at least about 95%, at least about 97%, or at least about 99% sequence identity to the amino acid sequence of SEQ ID NO: 4, 8, 13, or 15 across the full length of the amino acid sequence, respectively.

In a separate embodiment, in the immunogenic conjugate of any one of embodiments 36-49, the capture sequence is selected from SpyTag, SpyTag001, SpyTag002, SpyTag003, SpyCatcher, SpyCatcher001, SpyCatcher002, SpyCatcher003, SnoopTag, SnoopCatcher, a sortase recognition domain, a sortase bridging domain, a butelase recognition motif, a C-peptide, a split intein, or a peptiligase substrate.

In a separate embodiment, in the immunogenic conjugate of any one of embodiments 36-50:
(a) the tether sequence is a SpyCatcher and the capture sequence is SpyTag;
(b) the tether sequence comprises the amino acid sequence of SEQ ID NO: 4 and the capture sequence comprises the amino acid sequence of SEQ ID NO: 13;
(c) the tether sequence is a SpyTag and the capture sequence is SpyCatcher;
(d) the tether sequence comprises the amino acid sequence of SEQ ID NO: 13 and the capture sequence comprises the amino acid sequence of SEQ ID NO: 4;
(e) the tether sequence is a sortase Tag and the capture sequence is sortase A;
(f) the tether sequence is a sortase A and the capture sequence is sortase Tag;
(g) the tether sequence comprises the amino acid sequence of SEQ ID NO: 8 and the capture sequence comprises the amino acid sequence of SEQ ID NO: 15;
(h) the tether sequence comprises the amino acid sequence of SEQ ID NO: 15 and the capture sequence comprises the amino acid sequence of SEQ ID NO: 8.

In a separate embodiment, in the immunogenic conjugate of any one of embodiments 36-51, the self-assembling nanoparticle further comprises a linker.

In a separate embodiment, in the immunogenic conjugate of embodiment 52, the linker is a glycine-serine linker or the linker comprises an amino acid sequence of SEQ ID NO: 5 or 9 or an amino acid sequence having at least about 70%, at least about 75%, at least about 80%, at least about 85%, at least about 90%, at least about 95%, at least about 97%, or at least about 99% identity to the amino acid sequence of SEQ ID NO: 5 or 9 across the full length of the amino acid sequence, respectively.

In a separate embodiment, in the immunogenic conjugate of any one of embodiments 37-53, the purification tag is selected from hexahistidine tag (SEQ ID NO: 65) (his-tag), Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin).

In a separate embodiment, in the immunogenic conjugate of any one of embodiments 37-54, the purification tag comprises an amino acid sequence having at least about 70%, at least about 75%, at least about 80%, at least about 85%, at least about 90%, at least about 95%, at least about 97%, or at least about 99% identity to the amino acid sequence of SEQ ID NO: 6 across the full length of the amino acid sequence.

In a separate embodiment, in the immunogenic conjugate of any one of embodiments 37-55, the purification tag comprises the amino acid sequence of SEQ ID NO: 6.

In a separate embodiment, in the immunogenic conjugate of any one of embodiments 36-56, the protein of interest is selected from an antigen, a tumor antigen, a viral antigen, a fungal antigen, a bacterial antigen, an antigen for the development of a vaccine, an antibody, an antibody fragment, a single chain antibody, scFv, scFab, single domain antibody, a protein scaffolds, an enzyme, a hormone, or an interleukin.

In a separate embodiment, in the immunogenic conjugate of any one of embodiments 36-57, the protein of interest is a viral antigen selected from:
(a) a coronavirus;
(b) SARS-CoV-1, MERS-CoV, 229E (alpha), NL63 (alpha), OC43 (beta), HKU1 (beta), or SARS-CoV-2; or
(c) a SARS-CoV-2 variant selected from B.1.1.7, B.1.1.7 with E484K, B.1.135, B.1.351, P.1, B.1.427, D614G, B.1.1351, B.1.429, Lambda (C.37), or Mu (B.1.621).

In a separate embodiment, in the immunogenic conjugate of any one of embodiments 36-58, the protein of interest is:
(a) a coronavirus spike protein;
(b) a mutant coronavirus spike protein;
(c) a mutant coronavirus spike protein comprising 1, 2, 3, 4, or 5 proline mutations selected from F817P, A892P, A899P, A942P, P986K, K986P, V987P, and P987V; and/or
(d) a mutant coronavirus spike protein comprising at least one additional disulfide bond selected from F43C-G566C, G413C-P987C, Y707C-T883C, G1035C-V1040C, A701C-Q787C, G667C-L864C, V382C-R983C, and I712C-I816C.

In a separate embodiment, in the immunogenic conjugate of embodiment 59, the mutant coronavirus spike protein comprises the amino acid sequence of SEQ ID NO: 11 or an amino acid sequence having at least about 70%, at least about 75%, at least about 80%, at least about 85%, at least about 90%, at least about 95%, at least about 97%, or at least about 99% identity to the amino acid sequence of SEQ ID NO: 11 across the full length of the amino acid sequence.

In a separate embodiment, in the immunogenic conjugate of any one of embodiment 36-60, the protein of interest is operably linked to a purification tag, and/or a trimerization motif.

In a separate embodiment, in the immunogenic conjugate of embodiment 61, the trimerization motif is a T4 fibritin foldon, an HIV-I -derived molecular clamp, or a viral capsid protein SHP.

In a separate embodiment, in the immunogenic conjugate of embodiment 61 or 62, the trimerization motif comprises the amino acid sequence of SEQ ID NO: 12, 21, or 22 or an amino acid sequence having at least about 70%, at least about 75%, at least about 80%, at least about 85%, at least about 90%, at least about 95%, at least about 97%, or at least about 99% identity to the amino acid sequence of SEQ ID NO: 12, 21, or 22 across the full length of the amino acid sequence, respectively.

In a separate embodiment, in the immunogenic conjugate of any one of embodiments 38-63, the T-cell epitope:
(a) is selected from a universal DR epitope (PADRE) T-helper epitope, a CpG-oligodeoxynucleotides (CpG-ODNs), a multi-epitope long peptide, a SARS-CoV-2 nucleo capsid protein N-terminal domain, a SARS-CoV-2 nucleoprotein T-cell epitope;
(b) comprises the amino acid sequence of SEQ ID NO: 23-27 or an amino acid sequence having at least about 70%, at least about 75%, at least about 80%, at least about 85%, at least about 90%, at least about 95%, at least about 97%, or at least about 99% identity to the amino acid sequence of SEQ ID NO: 23-27 across the full length of the amino acid sequence, respectively; or
(c) a SARS-CoV-2 nucleoprotein epitope comprising the amino acid sequence of SEQ ID NO: 26 or 27.

In a separate embodiment, provided herein is a nucleic acid encoding the self-assembly nanoparticle of any one of embodiments 1-30, or the immunogenic conjugate of any one of embodiments 36-64.

In a separate embodiment, provided herein is an immunogenic composition comprising the immunogenic conjugate of any one of embodiments 36-64.

In a separate embodiment, in the immunogenic composition of embodiment 66, wherein the immunogenic conjugate comprises from N-terminus to C-Terminus:
(a) a polypeptide of an antigen of interest, a recombined tether/capture sequence, a glycine linker, and a self-assembly KARI nanoparticle sequence;
(b) a polypeptide of an antigen of interest, a trimerization domain, a recombined tether/capture sequence, a glycine linker, and a self-assembly KARI nanoparticle sequence;
(c) a polypeptide of an antigen of interest, a trimerization domain, a recombined tether/capture sequence, a glycine linker, a self-assembly KARI nanoparticle sequence, and a T-cell epitope; or
(d) a polypeptide of an antigen of interest, a recombined tether/capture sequence, a glycine linker, a self-assembly KARI nanoparticle sequence, and a T-cell epitope.

In a separate embodiment, in the immunogenic composition of embodiment 66, or 67, the immunogenic conjugate comprises from N-terminus to C-Terminus:
(a) a polypeptide of an antigen of interest of SEQ ID NO: 11, a glycine linker of SEQ ID NOs: 5, 9, or 16-20, and a self-assembly KARI nanoparticle sequence of SEQ ID NO: 1 or 2;
(b) a polypeptide of an antigen of interest of SEQ ID NO: 11, a trimerization domain of SEQ ID NO: 21 or 22, a glycine linker of SEQ ID NOs: 5, 9, or 16-20, and a self-assembly KARI nanoparticle sequence of SEQ ID NO: 1 or 2;
(c) a polypeptide of an antigen of interest of SEQ ID NO: 11, a trimerization domain of SEQ ID NO: 21 or 22, a glycine linker a glycine linker f SEQ ID NOs: 5, 9, or 16-20, a self-assembly KARI nanoparticle sequence of SEQ ID NO: 1 or 2, and a T-cell epitope of SEQ ID NO: 22-27; or
(d) a polypeptide of an antigen of interest of SEQ ID NO: 11, a glycine linker of SEQ ID NOs: 5, 9, or 16-20, a self-assembly KARI nanoparticle sequence of SEQ ID NO: 1 or 2, and a T-cell epitope of SEQ ID NO: 22-27.

In a separate embodiment, in the immunogenic composition of any one of embodiments 66-68, the immunogenic conjugate further comprises a purification tag, optionally wherein the purification tag is operably linked to a C-terminus or an N-terminus of the recombinant KARI.

In a separate embodiment, provided herein is a pharmaceutical composition, comprising the immunogenic composition of any one of embodiments 66-69, and a pharmaceutically acceptable carrier.

In a separate embodiment, provided herein is a method for preventing or treating a disease in a subject, comprising administering to the subject a pharmaceutically effective amount of the immunogenic composition of any one of embodiments 66-69 or the pharmaceutical composition of embodiment 70.

In a separate embodiment, provided herein is a method for generating an immune response to a protein of interest in a subject, comprising administering to the subject an effective amount of the immunogenic conjugate of any one of embodiments 36-64, the immunogenic composition of any one of embodiments 66-69, or the composition of embodiment 70 to generate the immune response.

In a further aspect, in the method of embodiment 72, the immune response treats or inhibits an infection or a disease progression in the subject.

In a separate embodiment, in the method of embodiment 72 or 73, wherein the administration of the immunogenic conjugate or immunogenic composition to the subject primes a protective immune response to an infection or a condition triggered by the protein of interest in the subject.

In a separate embodiment, provided herein is a diagnostic agent comprising the self-assembly nanoparticle of any one of embodiments 1-30, or the immunogenic conjugate of any one of embodiments 36-64.

In a separate embodiment, provided herein is the use of the self-assembly nanoparticle of any one of embodiments 1-30, or the immunogenic conjugate of any one of embodiments 36-64 as a diagnostic agent.

In a separate embodiment, provided herein is a method of cell sorting comprising:
(a) introducing the self-assembly nanoparticle of any one of embodiments 1-30, or the immunogenic conjugate of any one of embodiments 36-64 into a cell sorting apparatus comprising a population of cells;
(b) allowing the self-assembly nanoparticle or the immunogenic conjugate to bond with specific type of cells, and
(c) sorting cells based on said bonding.

In a separate embodiment, provided herein is a method of imaging a target material comprising:
(a) introducing the self-assembly nanoparticle of any one of embodiments 1-30, or the immunogenic conjugate of any one of embodiments 36-64 into a medium containing said target material
(b) allowing the self-assembly nanoparticle or the immunogenic conjugate to bond with the target material; and
(c) obtaining an image of the target based on bonding between the self-assembly nanoparticle or the immunogenic conjugate and the target.

In a separate embodiment, in the method of embodiment 77 or 78, the protein of interest is:
(a) an antibody, an antibody fragment, a single chain antibody, scFv, scFab, single domain antibody, a protein scaffolds, an enzyme, a hormone, or an interleukin;
(b) a diagnostic agent; or
(c) an imaging agent.

### EQUIVALENTS

The present technology is not to be limited in terms of the embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to methods, reagents, compounds compositions or biological systems, which can, of course, vary. It is also to be understood that the terminology used herein is for the purpose of describing embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for all purposes, particularly in terms of providing a written description, all ranges disclosed herein also encompass all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All publications, patents, and patent applications mentioned in this specification are herein incorporated by reference in their entireties to the same extent as if each individual publication, patent, or patent application was specifically and individually indicated to be incorporated by reference.

### REFERENCES

Chen, Chin-Yu, Yuan-Chih Chang, Bo-Lin Lin, Chun-Hsiang Huang, and Ming-Daw Tsai. 2019. “Temperature-Resolved Cryo-EM Uncovers Structural Bases of Temperature-Dependent Enzyme Functions.” Journal of the American Chemical Society 141 (51): 19983-87.
Chen, Chin-Yu, Yuan-Chih Chang, Bo-Lin Lin, Kuan-Fu Lin, Chun-Hsiang Huang, Dong-Lin Hsieh, Tzu-Ping Ko, and Ming-Daw Tsai. 2019. “Use of Cryo-EM To Uncover Structural Bases of pH Effect and Cofactor Bispecificity of Ketol-Acid Reductoisomerase.” Journal of the American Chemical Society 141 (15): 6136-40.
Li, Long, Jacob O. Fierer, Tom A. Rapoport, and Mark Howarth. 2014. “Structural Analysis and Optimization of the Covalent Association between SpyCatcher and a Peptide Tag.” Journal of Molecular Biology. https://doi.org/10.1016/j.jmb.2013.10.021.
Terao, Yutaka, Shigetada Kawabata, Masanobu Nakata, Ichiro Nakagawa, and Shigeyuki Hamada. 2002. “Molecular Characterization of a Novel Fibronectin-Binding Protein of Streptococcus Pyogenes Strains Isolated from Toxic Shock-like Syndrome Patients.” The Journal of Biological Chemistry 277 (49): 47428-35.
Zakeri, Bijan, Jacob O. Fierer, Emrah Celik, Emily C. Chittock, Ulrich Schwarz-Linek, Vincent T. Moy, and Mark Howarth. 2012. “Peptide Tag Forming a Rapid Covalent Bond to a Protein, through Engineering a Bacterial Adhesin.” Proceedings of the National Academy of Sciences of the United States of America 109 (12): E690-97.

1. A self-assembling nanoparticle comprising an amino acid sequence of a recombinant Ketol-acid reductoisomerase (KARI; EC 1.1.1.86) and a tether sequence operably linking the KARI to at least one protein or peptide of interest wherein each protein comprises a capture sequence.

2. The self-assembling nanoparticle of claim 1, further comprising
(a) a purification tag, optionally wherein the purification tag is operably linked to a C-terminus or an N-terminus of the recombinant KARI;
(b) a T-cell epitope, operably linked to the C-terminus or the N-terminus of the recombinant KARI; or
(c) a linker.

3.-30. (canceled)

31. A combination of vectors comprising the self-assembling nanoparticle of claim 2, wherein the combination of vectors comprises:
(a) a first vector comprising a nucleic acid sequence encoding the recombinant KARI, and a nucleic acid sequence encoding the tether sequence; or
(b) a first vector comprising a nucleic acid sequence encoding the recombinant KARI, a nucleic acid sequence encoding the tether sequence, and a nucleic acid encoding the purification Tag; and
(c) a second vector comprising a nucleic acid sequence encoding a protein of interest and a nucleic acid sequence encoding the capture sequence.

32. A combination of vectors comprising the self-assembling nanoparticle of claim 2, wherein the combination of vectors comprises:
(a) a first vector comprising a nucleic acid sequence encoding the recombinant KARI, a nucleic acid sequence encoding the tether sequence, and a nucleic acid encoding the T-cell epitope; or
(b) a first vector comprising a nucleic acid sequence encoding the recombinant KARI, a nucleic acid sequence encoding the tether sequence, a nucleic acid encoding the T-cell epitope, and a nucleic acid encoding the purification Tag; and
(c) a second vector comprising a nucleic acid sequence encoding a protein of interest and a nucleic acid sequence encoding the capture sequence.

33. The vector of claim 31, wherein:
(a) the first vector is expressed in a bacterial cell and the second vector is expressed in a mammalian cell line;
(b) the first vector encodes a polypeptide having the amino acid sequence of SEQ ID NO: 3 or 7 and the second vector encodes a polypeptide having the amino acid sequence of SEQ ID NO: 10 or 14; and/or
(c) when the first and second vectors are brought together, the tether sequence and the capture sequence form a covalent bond with one another either spontaneously or with the help of an enzyme to generate an immunogenic polypeptide comprising the self-assembling nanoparticle fused to the protein of interest.

34.-35. (canceled)

36. An immunogenic conjugate comprising:
(a) a self-assembling nanoparticle comprising an amino acid sequence of a recombinant Ketol-acid reductoisomerase (KARI; EC 1.1.1.86), and a tether sequence;
(b) a protein of interest comprising a capture sequence; wherein the capture sequence and the tether sequence form a covalent bond that operably links the self-assembling nanoparticle to the protein of interest.

37. The immunogenic conjugate of claim 36, further comprising:
(a) a purification tag, optionally wherein the purification tag is operably linked to a C-terminus or an N-terminus of the recombinant KARI; or
(b) a T-cell epitope, optionally wherein the T-cell epitope is operably linked to the C-terminus or the N-terminus of the recombinant KARI.

38.-39. (canceled)

40. The immunogenic conjugate of claim 36, wherein the recombinant KARI:
(a) is an archeal, bacterial, or proteobacteria KARI;
(b) is selected from Methanothermococcus thermolithotrophicus (MtKARI), Helicobacter pylori, Pseudomonas Aeruginosa (PaKARI), Saccharolobus solfataricus (SacsKARI), Sulfolobus solfataricus (Sso-KARI), or A. vinelandii, Sulfolobus sp. E5-1-F, Sulfolobus islandicus, Saccharolobus shibatae, Saccharolobus caldissimus, Saccharolobus shibatae, Stygiolobus sp. KN-1, Sulfolobus sp., Sulfodiicoccus acidiphilus, Acidianus brierleyi, or Acidianus manzaensis;
(c) is a dodecameric KARI selected from M. thermolithotrophicus (MtKARI), Helicobacter pylori, Pseudomonas, Aeruginosa (PaKARI), Saccharolobus solfataricus (SacsKARI), Sulfolobus solfataricus (Sso-KARI), or A. vinelandii;
(d) oligomerizes into a dodecameric (12-mer) nanoparticle; or
(e) oligomerizes into a dodecameric KARI nanoparticle and displays the protein of interest on a surface of the nanoparticle.

41.-43. (canceled)

44. The immunogenic conjugate of claim 36, wherein the recombinant KARI comprises the amino acid sequence of SEQ ID NO: 1 or 2, or an amino acid sequence having at least about 37%, at least about 40%, at least about 50%, at least about 55%, at least about 65%, at least about 70%, at least about 75%, at least about 80%, at least about 85%, at least about 90%, at least about 95%, or at least about 99% identity to the amino acid sequence of SEQ ID NO: 1 or 2 across the full length of the amino acid sequence, respectively.

45.-47. (canceled)

48. The immunogenic conjugate of claim 36, wherein the recombinant KARI comprises:
(a) a deletion in the amino acid sequence of SEQ ID NO: 1; or
(b) a deletion of about 2, about 3, about 5, about 10, about 12, about 15, or about 20 amino acids in the N-terminus of SEQ ID NO: 1,
optionally wherein the deletion enhances the expression and purification of the self-assembly nanoparticle when compared to the expression and purification of a wild-type KARI nanoparticle.

49. The immunogenic conjugate of claim 36, wherein the tether sequence or the capture sequence:
(a) is selected from SpyTag, SpyTag001, SpyTag002, SpyTag003, SpyCatcher, SpyCatcher001, SpyCatcher002, SpyCatcher003, SnoopTag, SnoopCatcher, a sortase recognition domain, a sortase bridging domain, a butelase recognition motif, a C-peptide, a split intein, or a peptiligase substrate; or
(b) comprises the amino acid sequence of SEQ ID NO: 4, 8, 13, or 15 or an amino acid sequence having at least about 70%, at least about 75%, at least about 80%, at least about 85%, at least about 90%, at least about 95%, at least about 97%, or at least about 99% sequence identity to the amino acid sequence of SEQ ID NO: 4, 8, 13, or 15 across the full length of the amino acid sequence, respectively.

50. (canceled)

51. The immunogenic conjugate of claim 36, wherein:
(a) the tether sequence is a SpyCatcher and the capture sequence is SpyTag;
(b) the tether sequence comprises the amino acid sequence of SEQ ID NO: 4 and the capture sequence comprises the amino acid sequence of SEQ ID NO: 13;
(c) the tether sequence is a SpyTag and the capture sequence is SpyCatcher;
(d) the tether sequence comprises the amino acid sequence of SEQ ID NO: 13 and the capture sequence comprises the amino acid sequence of SEQ ID NO: 4;
(e) the tether sequence is a sortase Tag and the capture sequence is sortase A;
(f) the tether sequence is a sortase A and the capture sequence is sortase Tag;
(g) the tether sequence comprises the amino acid sequence of SEQ ID NO: 8 and the capture sequence comprises the amino acid sequence of SEQ ID NO: 15; or
(h) the tether sequence comprises the amino acid sequence of SEQ ID NO: 15 and the capture sequence comprises the amino acid sequence of SEQ ID NO: 8.

52.-56. (canceled)

57. The immunogenic conjugate of claim 37, wherein:
(a) the protein of interest is selected from an antigen, a tumor antigen, a viral antigen, a fungal antigen, a bacterial antigen, an antigen for the development of a vaccine, an antibody, an antibody fragment, a single chain antibody, scFv, scFab, single domain antibody, a protein scaffolds, an enzyme, a hormone, or an interleukin;
(b) the protein of interest is operably linked to a purification tag, and/or a trimerization motif; or
(c) the T-cell epitope:
(i) is selected from a universal DR epitope (PADRE) T-helper epitope, a CpG-oligodeoxynucleotides (CpG-ODNs), a multi-epitope long peptide, a SARS-CoV-2 nucleo capsid protein N-terminal domain, a SARS-CoV-2 nucleoprotein T-cell epitope;
(ii) comprises the amino acid sequence of SEQ ID NO: 23-27 or an amino acid sequence having at least about 70%, at least about 75%, at least about 80%, at least about 85%, at least about 90%, at least about 95%, at least about 97%, or at least about 99% identity to the amino acid sequence of SEQ ID NO: 23-27 across the full length of the amino acid sequence, respectively; or
(iii) a SARS-CoV-2 nucleoprotein epitope comprising the amino acid sequence of SEQ ID NO: 26 or 27.

58.-61. (canceled)

62. The immunogenic conjugate of claim 57, wherein the trimerization motif:
(a) is a T4 fibritin foldon, an HIV-I-derived molecular clamp, or a viral capsid protein SHP; or
(b) comprises the amino acid sequence of SEQ ID NO: 12, 21, or 22 or an amino acid sequence having at least about 70%, at least about 75%, at least about 80%, at least about 85%, at least about 90%, at least about 95%, at least about 97%, or at least about 99% identity to the amino acid sequence of SEQ ID NO: 12, 21, or 22 across the full length of the amino acid sequence, respectively.

63.-64. (canceled)

65. A nucleic acid encoding the immunogenic conjugate of claim 36.

66. An immunogenic composition comprising the immunogenic conjugate of claim 36.

67. The immunogenic composition of claim 66, wherein the immunogenic conjugate comprises from N-terminus to C-terminus:
(a) a polypeptide of an antigen of interest, a recombined tether/capture sequence, a glycine linker, and a self-assembly KARI nanoparticle sequence;
(b) a polypeptide of an antigen of interest, a trimerization domain, a recombined tether/capture sequence, a glycine linker, and a self-assembly KARI nanoparticle sequence;
(c) a polypeptide of an antigen of interest, a trimerization domain, a recombined tether/capture sequence, a glycine linker, a self-assembly KARI nanoparticle sequence, and a T-cell epitope; or
(d) a polypeptide of an antigen of interest, a recombined tether/capture sequence, a glycine linker, a self-assembly KARI nanoparticle sequence, and a T-cell epitope.

68. The immunogenic composition of claim 66, wherein the immunogenic conjugate comprises from N-terminus to C-terminus:
(a) a polypeptide of an antigen of interest of SEQ ID NO: 11, a glycine linker of SEQ ID NOs: 5, 9, or 16-20, and a self-assembly KARI nanoparticle sequence of SEQ ID NO: 1 or 2;
(b) a polypeptide of an antigen of interest of SEQ ID NO: 11, a trimerization domain of SEQ ID NO: 21 or 22, a glycine linker of SEQ ID NOs: 5, 9, or 16-20, and a self-assembly KARI nanoparticle sequence of SEQ ID NO: 1 or 2;
(c) a polypeptide of an antigen of interest of SEQ ID NO: 11, a trimerization domain of SEQ ID NO: 21 or 22, a glycine linker a glycine linker f SEQ ID NOs: 5, 9, or 16-20, a self-assembly KARI nanoparticle sequence of SEQ ID NO: 1 or 2, and a T-cell epitope of SEQ ID NO: 22-27; or
(d) a polypeptide of an antigen of interest of SEQ ID NO: 11, a glycine linker of SEQ ID NOs: 5, 9, or 16-20, a self-assembly KARI nanoparticle sequence of SEQ ID NO: 1 or 2, and a T-cell epitope of SEQ ID NO: 22-27.

69. (canceled)

70. A pharmaceutical composition, comprising the immunogenic composition of claim 66, and a pharmaceutically acceptable carrier.

71. A method for preventing or treating a disease in a subject, comprising administering to the subject a pharmaceutically effective amount of the pharmaceutical composition of claim 70.

72.-79. (canceled)
